(19) United States
(12) Patent Application Publication
(10) Pub. No.: US 2025/0357012 A1
(43) Pub. Date: Nov. 20, 2025

(54) SYSTEMS AND METHODS FOR PROVIDING CONTEXT SENSITIVE GUIDANCE FOR MEDICAL TREATMENT OF A PATIENT

(71) Applicant: ZOLL Medical Corporation, Chelmsford, MA (US)

(72) Inventors: Paolo Giacometti, North Grafton, MA (US); George Beck, Salem, MA (US); Andrew Kirkpatrick, Calgary, Alberta (CA); Jessica L. McKee, Edmonton, Alberta (CA); Guy R. Johnson, Wilton, NH (US); Frederick W. Forester, Encinitas, CA (US); Gary A. Freeman, Waltham, MA (US); Lisa M. Campana, Waltham, MA (US)

(73) Assignee: ZOLL Medical Corporation, Chelmsford, MA (US)

(21) Appl. No.: 18/993,168

(22) PCT Filed: Jul. 13, 2023
(86) PCT No.: PCT/US2023/070093

§ 371 Date: Jan. 10, 2025

Related U.S. Application Data
(60) Provisional application No. 63/368,402, filed on Jul. 14, 2022.

(51) Int. Cl. G16H 70/20; A61N 1/39

(52) U.S. Cl. CPC G16H 70/20; A61N 1/3993

(57) ABSTRACT

An example of a context sensitive guidance (CSG) system for guiding caregivers providing medical care for a victim, for example a trauma victim, includes a CSG engine and contextual data sources communicatively coupled to the CSG engine. The contextual data sources include a medical device configured to collect physiologic data and provide the physiologic data to the CSG engine and an emergency environment data source configured to receive emergency environment data and provide the data to the CSG engine. The CSG engine includes hardware logic and/or software logic configured to receive the contextual data, evaluate protocols based on the contextual data, select at least one action item based on the protocols, generate at least one instruction based on the action item(s), the instruction(s) including a caregiver instruction and/or a medical device instruction, and provide the instruction to a caregiver interface device and/or the medical device.

### BACKGROUND

In a pre-hospital and/or acute care treatment setting, medical responders often have difficulty in accurately determining the most effective medical interventions for a patient. In these settings, split second decisions about interventions for emergency conditions such as respiratory distress, cardiac arrest, and/or trauma are often required. Worldwide, traumatic injury is the leading cause of lost years of life. In many cases, death results from the fact that a potentially treatable injury is not recognized and treated quickly enough. A common example of this case is exsanguination or severe blood loss. If recognized and treated promptly, death or permanent injury due to trauma induced blood loss is preventable.

One challenge in an emergency trauma response is that often multiple physiologic systems are injured during the event. For example, a victim of a car collision may have several broken ribs, a spinal injury, and a concussion. Further, one of the broken ribs may cause internal bleeding and damage to a lung. Another challenge is that a treatment for one injury may exacerbate another. For example, if chest compressions are provided to a patient that has crashed the car due to a cardiac arrest, those chest compressions could dangerously exacerbate bleeding. A further challenge is that an improperly applied treatment may cause a delayed and/or dangerous reaction. For example, a caregiver may improperly apply a tourniquet and it may slowly loosen leading to a resumption of bleeding and a drop in blood pressure.

These medical challenges are further complicated by the fact that often the pre-hospital or acute care treatment environment is noisy, crowded, and chaotic and may be temporary. The car collision described above may be at the side of a busy highway during a snowstorm and involve multiple victims. The caregivers in such a situation have to stabilize the victim sufficiently to transport them to a hospital for longer term care. In such settings, even well-trained paramedics, nurses, or physicians often have difficulty in rapidly assessing and recognizing patient conditions and in identifying and providing the most effective immediate interventions.

### SUMMARY

The foregoing general description of the illustrative implementations and the following detailed description thereof are merely exemplary aspects of the teachings of this disclosure and are not restrictive.

An example of a context sensitive guidance (CSG) system for guiding caregivers providing medical care for a victim includes a CSG engine including hardware logic and/or software logic and a plurality of contextual data sources configured to communicatively couple to the CSG engine. The contextual data sources include at least one medical device configured to collect physiologic data for the victim during the medical care, provide the physiologic data to the CSG engine and at least one emergency environment data source configured to receive emergency environment data during the medical care, and provide the emergency environment data to the CSG engine. The CSG engine is configured to receive contextual data including the physiologic data and the emergency environment data from the plurality of contextual data sources, evaluate a plurality of protocols based on the contextual data, select at least one action item for the medical care based on the plurality of protocols, generate at least one instruction based on the at least one action item, the at least one instruction including one or more of a caregiver instruction and a medical device instruction, and provide the at least one instruction to one or more of a caregiver interface device and the at least one medical device. In one or more examples, the at least one emergency environment data source is configured to receive emergency environment data prior to and/or during the medical care.

Implementations of such a system may include one or more of the following features. The CSG engine may be a trauma CSG engine. The victim may be a trauma victim. The plurality of protocols may be a plurality of trauma protocols or may include at least one trauma protocol. The at least one emergency environment data source may include a mobile device. The mobile device may include a computer tablet or a smartphone. The mobile device may be configured to provide a CSG user interface (UI) at a touchscreen display disposed at the computer tablet or the smartphone. The CSG UI may be a trauma CSG UI. The CSG UI may be configured to receive the emergency environment data as touchscreen input to the CSG UI. The touchscreen input may include a user selection of a menu item or a control at the CSG UI. The caregiver interface device may include the mobile device and the CSG engine may be configured to provide the caregiver instruction as a visual instruction at the CSG UI. The CSG engine may be configured to provide an alert window at the CSG UI. The alert window may include an immediate transport alert based at least in part on the contextual data The caregiver interface device may include a mobile device, which may be the same mobile device that may be included as part of the at least one emergency environment data source or a different mobile device. The mobile device may include a heads-up device. The heads-up device may be configured to provide a CSG UI at a heads-up display. The mobile device may include a camera, a scanner, or a combination thereof. The CSG engine may be configured to receive the contextual data including one or more of an image from the camera and a barcode or a quick-response (QR) code information from the scanner or the camera. The mobile device may be communicatively coupled to a remote computing resource including one or more of a mobile device associated with a remotely located telemedicine provider, a medical records database, or a cloud computing service and the contextual data may include information received from the remote computing resource. The at least one emergency environment data source may include an earpiece. The contextual data may include voice data captured by a microphone disposed at the earpiece. The caregiver interface device may include the earpiece. The CSG engine may be configured to provide the caregiver instruction as an audible instruction from the earpiece. The at least one medical device may include at least one of a trauma kit, an automated compression device, a defibrillator, or a patient monitor. The at least one of a trauma kit, an automated compression device, a defibrillator, or a patient monitor may include or be communicatively coupled with one or more sensors and/or one or more electrodes configured to collect the physiologic data for the victim. The defibrillator may include an advanced life support (ALS) defibrillator coupled wirelessly to a companion mobile device. The companion mobile device may be a tablet computing device that may be pre-configured to communicatively couple with the ALS defibrillator and to provide a view of a user interface of the ALS defibrillator in real-time at a display screen disposed at the tablet computing device. The companion mobile device may be the caregiver interface device. The automated compression device may be a belt-based automated compression device. The trauma kit may include an integrated computer tablet. The trauma kit may be configured to provide medical supply inventory information to the CSG engine. The medical supply inventory information may be indicative of supplies used for trauma treatment that have been removed from the trauma kit in a patient care environment. The at least one medical device may include an ultrasound imaging device. The CSG engine may include a natural language processing (NLP) engine configured to receive the contextual data as unstructured data and convert the unstructured data to structured data including data elements associated with a care protocol. The care protocol may be a trauma care protocol. The CSG engine may be configured to select the at least one action item based at least in part on the structured data. The at least one emergency environment data source may include a microphone. The contextual data may include voice data. The voice data may include one or more of victim voice data and caregiver voice data. The NLP engine may be configured to receive the voice data as a text input from an automated speech to text engine. The contextual data may include sounds from an emergency environment. The contextual data may include one or more of camera data, scanner data, location data, and dispatch data. The contextual data may include information from a remotely located telemedicine provider. The NLP engine may be configured to create a curated transcript for a remotely located telemedicine provider based on the structured data. The physiologic data may include a textual input from the at least one medical device. The NLP engine may be configured to predict one or more items of future structured data based on previously determined structured data. The NLP engine may be configured to provide the at least one instruction as an audible instruction. The NLP engine may include at least one machine learning model associated with a plurality of protocols. The plurality of protocols may include trauma care protocols. The NLP engine may be configured to train and update the at least one machine learning model based on the contextual data. The CSG engine may be configured to communicatively couple to a cloud server. The at least one machine learning model may be a locally stored machine learning model in an unconnected state of the communicative coupling to the cloud server and may be a model stored at the cloud server in a connected state of the communicative coupling to the cloud server. The CSG engine may be configured to modify the at least one action item in response to a transition from the unconnected state to the connected state. The CSG engine may be configured to select the at least one action item based at least in part on an exclusion/inclusion criteria. The exclusion/inclusion criteria may be indicative of candidate patient conditions selected by a caregiver and/or of candidate patient conditions unselected by a caregiver. The CSG engine may configured to provide the candidate patient conditions in a list on a CSG UI. The CSG engine may be configured to monitor a network connectivity status between the CSG engine and a remote communications network, the network connectivity status including one of a connected state or an unconnected state. The CSG engine may be configured to communicatively couple to one or more of a remote telemedicine provider and a remote medical records database in the connected state. The CSG engine may be configured to evaluate the plurality of protocols without assigning an order of priority to the plurality of protocols, select the at least one action item from the plurality of protocols based on the contextual data wherein each protocol corresponds to a respective identified next step, determine at least one next best step from the plurality of next possible steps, and select the at least one action item based on the at least one next best step. The CSG engine may be configured to determine an order of performance for the plurality of next possible steps, exclusive of the at least one next best step, and provide a plurality of instructions to the one or more of the caregiver interface device and the at least one medical device according to the order of performance. The contextual data may be first contextual data and the CSG engine may be configured to receive second contextual data, identify a first care state based on the first contextual data and the plurality of protocols, recognize a state change to a second care state based on the second contextual data, and modify the order of performance for the plurality of next possible steps based on the state change. The CSG engine may be configured to record the first care state, the second care state, and the state change. The CSG engine may be configured to identify an updated plurality of next possible steps from the plurality of protocols based on the second care state, replace the plurality of next possible steps with the updated plurality of next possible steps, determine at least one updated next best step from the updated plurality of next possible steps, and select the at least one action item based on the at least one updated next best step. The CSG engine may be configured to identify one or more unperformed steps from the plurality of next possible steps due to the replacement with the updated plurality of next possible steps, maintain a log of the one or more unperformed steps, and generate reminders for at least one of the one or more unperformed steps, and provide the reminders to one or more of the caregiver interface device and the at least one medical device. The CSG engine may be configured to verify a completion of the at least one next best step. The CSG engine may be configured to identify a detrimental change in a physiologic condition of the victim based on the contextual data, and provide the at least one instruction to correct the detrimental change to the one or more of the caregiver interface device and the at least one medical device. The CSG engine may be configured to identify a previously performed action item associated with the detrimental change in the physiologic condition, and modify an order of performance for the plurality of next possible steps in order to return to the previously performed action item. The CSG engine may be configured to provide at least one contextual data source window. The contextual data source window may include indications of contextual data sources communicatively coupled to the CSG engine. The at least one contextual data source window may include one or more of a connected devices window or a connected software window. The plurality of contextual data sources may include a transport environment data source and the contextual data may include transport environment data. The transport environment data may include an inventory of medical equipment. In one or more examples, the transport environment data source may be provided instead of the emergency environment data source and thus the CSG engine is configured to receive contextual data including the physiologic data and the transport environment data from the plurality of contextual data sources. The inventory of medical equipment may include medical supplies and medical devices associated with a transport environment. The CSG engine may be configured to generate a request for additional medical equipment based on the inventory of medical equipment and the contextual data. The transport environment data may include a caregiver skill record for one or more caregivers associated with the transport environment. The transport environment data may include the caregiver skill record cross-referenced with the inventory of medical equipment. The transport environment data may include location and navigation data. The plurality of contextual data sources may include an emergency dispatch service and the contextual data may include emergency event notification information received from the emergency dispatch service. The CSG engine may be configured to receive the emergency event notification information prior to the physiologic data and the emergency environment data, provide the emergency event notification information to the CSG engine prior to an arrival of the caregivers at a patient care environment, and select at least one pre-arrival action item prior to the arrival of the caregivers at the patient care environment. The plurality of protocols may include a bleeding protocol, an airway protocol, a breathing protocol, and a circulation protocol. The plurality of protocols may include a loss of consciousness (LOC) protocol. The plurality of protocols may include a rapid trauma assessment protocol and a focused trauma assessment protocol. The plurality of protocols may include a first plurality of trauma protocols selected by the CSG engine based on off-site emergency event information. The CSG engine may be configured to add and/or replace one or more of the first plurality of trauma protocols with one or more additional protocols to form a second plurality of trauma protocols based on the contextual data. The CSG engine may be configured to identify a potential diagnosis of a victim condition based on the contextual data, determine a probability associated with the potential diagnosis, and select the at least one action item based on the potential diagnosis and the probability. The CSG engine may be configured to identify a transport location for the victim based on the contextual data, and select the at least one action item based on the transport location. The CSG engine may be configured to operate in an absence of a network connection between the CSG engine and a remote computing device. The CSG engine may be configured to communicatively couple to a computing device associated with a telemedicine provider, receive information from the telemedicine provider, and evaluate the plurality of protocols based on the information from the telemedicine provider. The CSG engine may be communicatively coupled to a patient charting application and the CSG engine may be configured to receive data from and provide data to the patient charting application. The CSG engine may be configured to receive emergency event notification information prior to an arrival of the caregivers at an emergency scene. The CSG engine may be configured to receive the emergency event notification information via caregiver touchscreen input to the CSG engine. The CSG engine may be communicatively coupled to a computer aided dispatch (CAD) system and may be configured to receive the emergency event notification information from the CAD system. The emergency event notification information may include at least one of a mechanism of injury (MOI) or an emergency scene location. The CSG engine may be configured to generate at least one preliminary caregiver instruction prior to the arrival of the caregivers at the emergency scene based on one or more of the MOI or the emergency scene location. The CSG engine may include a trauma CSG engine.

An example of a context sensitive guidance (CSG) system for providing CSG to caregivers providing medical care for a victim includes a CSG engine including hardware logic and/or software logic, a plurality of contextual data sources configured to communicatively couple to the CSG engine and including at least one medical device configured to collect physiologic data for the victim during the medical care, and provide the physiologic data to the CSG engine, and at least one emergency environment data source including a mobile computing device configured to capture caregiver observations at a touchscreen disposed at the mobile computing device during the medical care, and provide the caregiver observations to the CSG engine. The CSG engine is configured to receive the physiologic data and the caregiver observations, evaluate a plurality of protocols based on the physiologic data and the caregiver observations, select at least one action item for the medical care based on the evaluation of the plurality of protocols, generate at least one caregiver instruction based on the at least one action item, and provide the at least one caregiver instruction to the mobile computing device for display at the touchscreen.

Implementations of such a system may include one or more of the following features. The CSG engine may be a trauma CSG engine. The victim may be a trauma victim. The plurality of protocols may include at least one trauma protocol. The at least one medical device may include a patient monitor/defibrillator and one or more of a digital stethoscope and an ultrasound imaging device. The at least one medical device may include at least one of a trauma kit, an automated compression device, a defibrillator, or a patient monitor. The defibrillator may include an advanced life support (ALS) defibrillator coupled wirelessly to a companion mobile device. The companion mobile device may be a tablet computing device that may be pre-configured to communicatively couple with the ALS defibrillator and to provide a view of a user interface of the ALS defibrillator in real-time at a display screen disposed at the tablet computing device. The companion mobile device may be the mobile computing device. The automated compression device may be a belt-based automated compression device. The trauma kit may include an integrated computer tablet. The trauma kit may be configured to provide medical supply inventory information to the CSG engine. The medical supply inventory information may be indicative of supplies used for trauma treatment that have been removed from the trauma kit in a patient care environment. The CSG engine may be configured to request medical device inventory information for a patient care environment from a caregiver at the touchscreen. The CSG engine may be configured to request caregiver skill information from the caregiver at the touchscreen. The CSG engine may be configured to access a previously stored medical device inventory for a patient care environment. The CSG engine may be configured to access a previously stored caregiver skill record for the patient care environment. The physiologic data may include an ultrasound image or ultrasound analytics. The caregiver observations may include stethoscope examination results. The plurality of protocols may include at least a pneumothorax protocol and a cardiac tamponade protocol. The at least one action item may include one of needle decompression, fluid administration, ventilation, or vasopressor administration. The plurality of protocols may include a bleeding protocol. The at least one action item may include one of a tourniquet application or a packing/spray foam administration. The CSG engine may be configured to provide a CSG user interface (UI) at the touchscreen, and provide instructions for the at least one action item at the CSG UI. The CSG UI may be a trauma CSG UI. The CSG UI may be configured to provide device view window for at least one medical device communicatively coupled to the CSG system. The device view window may include one or more source indicators that show a source of a particular item of information in the device view window. The CSG engine may be configured to provide guidance selection controls at the CSG UI in conjunction with the instructions for the at least one action item. The guidance selection controls enable a caregiver to select a level of detail of the provided instructions. The guidance selection controls may include at least one of a continue instructions control, an exit instructions control, a proceed to a next step control, an increase a detail level for guidance control, and a return to a previous instruction control, and a mute or unmute audible UI output control. The guidance selection controls may include a scrollable notification window. The CSG engine may be configured to receive a caregiver confirmation at the CSG UI in response to the instructions for the at least one action item. The CSG engine may be configured to receive an incomplete treatment explanation based on the instructions for the at least one action item. The instructions may include instructions for at least one of operation or assembly of a medical device. The instructions may include medical device settings. The CSG engine may be configured to provide a medication timer at the CSG UI. The CSG engine may be configured to provide medication delivery instructions with the medication timer. The CSG engine may be configured to provide closed loop control of at least one medical device based on the at least one action item. The mobile computing device may include a smartphone. The mobile computing device may include a watch communicatively coupled to the smartphone. The CSG engine may be configured to provide the CSG UI in response to a user selection of a CSG UI tab. The mobile computing device provides at least one of a device view window tab, a working view window tab, or a trend view window tab as alternatives to the CSG UI tab. The CSG engine may be configured to operate in an absence of a network connection between the mobile computing device and a remote computing device. The mobile computing device may be configured to communicatively couple to a computing device associated with a telemedicine provider. The CSG engine may be configured to receive information from the telemedicine provider and evaluate the plurality of protocols based on the information from the telemedicine provider. The mobile computing device may include a patient charting application, and the CSG engine may be configured to receive data from and provide data to the patient charting application. The CSG engine may be configured to provide a connected software window at a CSG UI. The connected software window may indicate a connection status of the patient charting application. The CSG engine may be configured to provide a code generator at a CSG UI. The code generator may be configured to generate one or more of a bar code or QR code comprising one or more of medication information, patient information, emergency event information, or device connectivity information. The CSG engine may be configured to receive emergency event notification information prior to an arrival of the caregivers at an emergency scene. The CSG engine may be configured to receive the emergency event notification information via caregiver input to the touchscreen. The mobile computing device may be communicatively coupled to a computer aided dispatch (CAD) system and may be configured to receive the emergency event notification information from the CAD system. The emergency event notification information may include at least one of a mechanism of injury (MOI) or an emergency scene location. The CSG engine may be configured to generate at least one preliminary caregiver instruction prior to the arrival of the caregivers at the emergency scene based on one or more of the MOI or the emergency scene location.

An example of a context sensitive guidance (CSG) system includes at least one non-transitory, processor-readable storage medium having stored thereon processor-readable instructions for guiding caregivers in providing medical care for a victim. The processor-readable instructions are configured to cause at least one processor to communicatively couple to a plurality of contextual data sources including at least one medical device and at least one emergency environment data source, receive contextual data comprising physiologic data for the victim from the at least one medical device and emergency environment data from the at least one emergency environment data source, evaluate a plurality of protocols based on the contextual data, select at least one action item for the medical care based on the plurality of protocols, generate at least one instruction based on the at least one action item, the at least one instruction comprising one or more of a caregiver instruction and a medical device instruction, and provide the at least one instruction to one or more of a caregiver interface device and the at least one medical device. In one or more examples, the at least one emergency environment data source is configured to receive emergency environment data prior to and/or during the medical care.

Implementations of such a system may include one or more of the following features. The plurality of protocols may include at least one trauma protocol. The at least one emergency environment data source may include a mobile device. The mobile device may include a computer tablet or a smartphone. The mobile device may be configured to provide a CSG user interface (UI) at a touchscreen display disposed at the computer tablet or the smartphone. The CSG UI may be a trauma CSG UI. The CSG UI may be configured to receive the emergency environment data as touchscreen input to the CSG UI. The touchscreen input may include a user selection of a menu item or a control at the CSG UI. The caregiver interface device may include the mobile device. The mobile device may be the same mobile device that may be included as part of the at least one emergency environment data source or a different mobile device. The processor-readable instructions may be configured to cause the at least one processor to provide the caregiver instruction as a visual instruction at the CSG UI. The processor-readable instructions may be configured to cause the at least one processor to provide an alert window at the CSG UI. The alert window may include an immediate transport alert based at least in part on the contextual data. The mobile device may include a heads-up device. The heads-up device may be configured to provide a CSG UI at the heads-up display. The mobile device may include a camera, a scanner, or a combination thereof. The processor-readable instructions may be configured to cause the at least one processor to receive the contextual data comprising one or more of an image from the camera and a barcode or a quick-response (QR) code information from the scanner or the camera. The mobile device may be communicatively coupled to a remote computing resource comprising one or more of a mobile device associated with a remotely located telemedicine provider, a medical records database, or a cloud computing service. The contextual data may include information received from the remote computing resource. The at least one emergency environment data source may include an earpiece. The contextual data may include voice data captured by a microphone disposed at the earpiece. The caregiver interface device may include the earpiece. The processor-readable instructions may be configured to cause the at least one processor to provide the caregiver instruction as an audible instruction from the earpiece. The at least one medical device may include at least one of a trauma kit, an automated compression device, a defibrillator, or a patient monitor. The defibrillator may include an advanced life support (ALS) defibrillator coupled wirelessly to a companion mobile device. The companion mobile device is a tablet computing device that is pre-configured to communicatively couple with the ALS defibrillator and to provide a view of a user interface of the ALS defibrillator in real-time at a display screen disposed at the tablet computing device. The companion mobile device may be the caregiver interface device. The automated compression device may be a belt-based automated compression device. The trauma kit may include an integrated computer tablet. The at least one processor may be configured to receive medical supply inventory information from the trauma kit. The medical supply inventory information may be indicative of supplies used for trauma treatment that have been removed from and/or remain in the trauma kit in a patient care environment. The at least one medical device may include an ultrasound imaging device. The processor-readable instructions may be configured to cause the at least one processor to receive the contextual data as unstructured data, convert the unstructured data to structured data comprising data elements associated with a care protocol, and select the at least one action item based at least in part on the structured data. The at least one emergency environment data source may include a microphone. The contextual data may include voice data. The voice data may include one or more of victim voice data and caregiver voice data. The processor-readable instructions may be configured to cause the at least one processor to receive the voice data and convert the voice data to text data. The contextual data may include sounds from an emergency environment. The contextual data may include one or more of camera data, scanner data, location data, and dispatch data. The contextual data may include information from a remotely located telemedicine provider. The processor-readable instructions may be configured to cause the at least one processor to create a curated transcript for a remotely located telemedicine provider based on the structured data. The physiologic data may include a textual input from the at least one medical device. The processor-readable instructions may be configured to cause the at least one processor to predict one or more items of future structured data based on previously determined structured data. The processor-readable instructions may be configured to cause the at least one processor to provide the at least one instruction as an audible instruction. The processor-readable instructions may be configured to cause the at least one processor to utilize at least one machine learning model associated with the plurality of protocols. The processor-readable instructions may be configured to cause the at least one processor to train and update the at least one machine learning model based on the contextual data. The processor-readable instructions may be configured to cause the at least one processor to communicatively couple to a cloud server. The at least one machine learning model may be a locally stored machine learning model in an unconnected state of the communicative coupling to the cloud server and may be stored at the cloud server in a connected state of the communicative coupling to the cloud server. The processor-readable instructions may be configured to cause the at least one processor to modify the at least one action item in response to a transition from the unconnected state to the connected state. The processor-readable instructions may be configured to cause the at least one processor to identify the plurality of next possible steps based at least in part on an exclusion/inclusion criteria. The exclusion/inclusion criteria may be indicative of candidate patient conditions selected by a caregiver and of candidate patient conditions unselected by a caregiver. The processor-readable instructions may be configured to cause the at least one processor to provide the candidate patient conditions in a list on a CSG UI. The processor-readable instructions may be configured to cause the at least one processor to monitor a network connectivity status with a remote communications network, the network connectivity status comprising one of a connected state or an unconnected state, and communicatively couple to one or more of a remote telemedicine provider and a remote medical records database in the connected state. The processor-readable instructions may be configured to cause the at least one processor to evaluate the plurality of protocols without assigning an order of priority to the plurality of protocols, identify a plurality of next possible steps from the plurality of protocols based on the contextual data wherein each protocol corresponds to a respective identified next step, determine at least one next best step from the plurality of next possible steps, and select the at least one action item based on the at least one next best step. The processor-readable instructions may be configured to cause the at least one processor to determine an order of performance for the plurality of next possible steps, exclusive of the at least one next best step, and provide a plurality of instructions to the one or more of the caregiver interface device and the at least one medical device according to the order of performance. The contextual data may be first contextual data. The processor-readable instructions may be configured to cause the at least one processor to receive second contextual data, identify a first care state based on the first contextual data and the plurality of protocols, recognize a state change to a second care state based on the second contextual data, and modify the order of performance for the plurality of next possible steps based on the state change. The processor-readable instructions may be configured to cause the at least one processor to record the first care state, the second care state, and the state change. The processor-readable instructions may be configured to cause the at least one processor to identify an updated plurality of next possible steps from the plurality of protocols based on the second care state, replace the plurality of next possible steps with the updated plurality of next possible steps, determine at least one updated next best step from the updated plurality of next possible steps, and select the at least one action item based on the at least one updated next best step. The processor-readable instructions may be configured to cause the at least one processor to identify one or more unperformed steps from the plurality of next possible steps due to the replacement with the updated plurality of next possible steps, maintain a log of the one or more unperformed steps, generate reminders for at least one of the one or more unperformed steps, and provide the reminders to one or more of the caregiver interface device and the at least one medical device. The processor-readable instructions may be configured to cause the at least one processor to verify a completion of the at least one next best step. The processor-readable instructions may be configured to cause the at least one processor to identify a detrimental change in a physiologic condition of the victim based on the contextual data, and provide the at least one instruction to correct the detrimental change to the one or more of the caregiver interface device and the at least one medical device. The processor-readable instructions may be configured to cause the at least one processor to identify a previously performed action item associated with the detrimental change in the physiologic condition, and modify an order of performance for the plurality of next possible steps in order to return to the previously performed action item. The processor-readable instructions may be configured to cause the at least one processor to provide at least one contextual data source window comprising indications of contextual data sources communicatively coupled to the at least one processor. The at least one contextual data source window may include one or more of a connected devices window or a connected software window. The plurality of contextual data sources may include a transport environment data source and the contextual data may include transport environment data. The transport environment data may include an inventory of medical equipment. The inventory of medical equipment may include medical supplies and medical devices associated with a transport environment. The processor-readable instructions may be configured to cause the at least one processor to generate a request for additional medical equipment based on the inventory of medical equipment and the contextual data. The transport environment data may include a caregiver skill record for one or more caregivers associated with the transport environment. The transport environment data may include the caregiver skill record cross-referenced with the inventory of medical equipment. The transport environment data may include location and navigation data. The plurality of contextual data sources may include an emergency dispatch service and the contextual data may include emergency event notification information received from the emergency dispatch service. The processor-readable instructions may be configured to cause the at least one processor to receive the emergency event notification information prior to the physiologic data and the emergency environment data, generate the emergency event notification information prior to an arrival of the caregivers at a patient care environment, and select at least one pre-arrival action item prior to the arrival of the caregivers at the patient care environment. The plurality of protocols comprise a bleeding protocol, an airway protocol, a breathing protocol, and a circulation protocol. The plurality of protocols may include a loss of consciousness (LOC) protocol. The plurality of protocols comprise a rapid trauma assessment protocol and a focused trauma assessment protocol. The plurality of protocols may include a first plurality of protocols selected by the at least one processor based on off-site emergency event information. The processor-readable instructions may be configured to cause the at least one processor to add and/or replace one or more of the first plurality of protocols with one or more additional protocols to form a second plurality of protocols based on the contextual data. The processor-readable instructions may be configured to cause the at least one processor to identify a potential diagnosis of a victim condition based on the contextual data, determine a probability associated with the potential diagnosis, and select the at least one action item based on the potential diagnosis and the probability. The processor-readable instructions may be configured to cause the at least one processor to identify a transport location for the victim based on the contextual data, and select the at least one action item based on the transport location. The processor-readable instructions may be configured to cause the at least one processor to operate in an absence of a network connection with a remote computing device. The processor-readable instructions may be configured to cause the at least one processor to communicatively couple to a computing device associated with a telemedicine provider, receive information from the telemedicine provider, and evaluate the plurality of protocols based on the information from the telemedicine provider. The processor-readable instructions may be configured to cause the at least one processor to communicatively couple to a patient charting application, and receive data from and provide data to the patient charting application. The processor-readable instructions may be configured to cause the at least one processor to receive emergency event notification information prior to an arrival of the caregivers at an emergency scene. The processor-readable instructions may be configured to cause the at least one processor to receive the emergency event notification information via caregiver touchscreen input. The processor-readable instructions may be configured to cause the at least one processor to communicatively coupled to a computer aided dispatch (CAD) system, and receive the emergency event notification information from the CAD system. The emergency event notification information may include at least one of a mechanism of injury (MOI) or an emergency scene location. The processor-readable instructions may be configured to cause the at least one processor to generate at least one preliminary caregiver instruction prior to the arrival of the caregivers at the emergency scene based on one or more of the MOI or the emergency scene location.

Other capabilities may be provided and not every implementation according to the disclosure must provide any, let alone all, of the capabilities discussed. Further, it may be possible for an effect noted above to be achieved by means other than that noted, and a noted item/technique may not necessarily yield the noted effect.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values and/or dimensions illustrated in the accompanying graphs and figures are for illustration purposes only and may or may not represent actual or preferred values or dimensions. Where applicable, some or all features may not be illustrated to assist in the description of underlying features.

FIG. 1 shows examples of emergency environments in which trauma may have occurred.

FIG. 2A shows an example of a CSG system deployed for treatment of victims of an emergency medical event in an emergency environment.

FIG. 2B shows examples of computing resources.

FIG. 2C shows examples of transport environment data and transport environment data sources.

FIG. 2D shows examples of medical equipment.

FIG. 3A shows an example of a device and data configuration of the CSG system in an emergency environment.

FIG. 3B shows examples of environmental data sources.

FIGS. 3C-1 and 3C-2 show examples of non-verbal data entry to the CSG engine.

FIG. 3D shows examples of non-verbal data entry to the CSG engine.

FIG. 3E illustrates audio input to the CSG engine.

FIG. 3F show examples of scanner and camera input to the CSG engine.

FIGS. 3G-1, 3G-2, and 3G-3 show examples of various configurations of the environmental data sources.

FIG. 4 shows an example of a CSG engine implemented on a computing device.

FIG. 5 shows an example of a CSG system configuration that includes a remote environment.

FIG. 6 shows an example of patient care environments within an emergency environment.

FIG. 7 shows an example of a method executed by a CSG system.

FIGS. 8A and 8B show examples of components and data sources for the CSG engine.

FIG. 9A shows an example of inputs and outputs to an AI modeling engine of a CSG engine.

FIG. 9B shows an example of a protocol loading engine.

FIG. 10 shows a schematic diagram of parallel protocol processing.

FIG. 11 shows an example of sequential protocol processing.

FIG. 12 shows a parallel protocol process for a CSG engine.

FIG. 13 shows an example of a process for CSG.

FIGS. 14A-1 and 14A-2 show examples of a CSG user interface.

FIG. 14A-3 shows an example of a manual device connection control.

FIGS. 14B-1, 14B-2, and 14B-3 show an example of a method of providing CSG at a user interface.

FIG. 14C shows an example of a UI population process for CSG.

FIGS. 14D-1, 14D-2, 14D-3, 14D-4, 14D5, 14D-6, 14D-7, and 14D-8 show examples of CSG UI features.

FIGS. 14E-1, 14E-2, and 14E-3 show examples of CSG UI features.

FIG. 15 is schematic diagram illustrating an example of an AI modeling engine of a CSG engine.

FIG. 16 is a schematic diagram illustrating an example of a mixed modality AI modeling engine of a CSG engine.

FIG. 17 shows a schematic illustration of an AI modeling engine with off-line modeling capabilities.

FIG. 18 illustrates an example of data flow diagram illustrating an NLP training system and process for a conversational user interface in accordance with examples of the present disclosure.

FIG. 19 shows an example environment for implementing CSG at a mobile device.

FIG. 20 illustrates an example method for configuring connection between a medical device and a mobile device.

FIG. 21 illustrates an example method for causing display of case information from a medical device at a mobile device during a medical event.

FIG. 22 shows schematic examples of components of various devices discussed herein.

### DETAILED DESCRIPTION

The description set forth below in connection with the appended drawings is intended to be a description of various, illustrative embodiments or implementations of the disclosed subject matter. Specific features and functionalities are described in connection with each illustrative embodiment or implementation; however, it will be apparent to those skilled in the art that the disclosed embodiments and implementations may be practiced without each of those specific features and functionalities.

Reference throughout the specification to “one embodiment,” “an embodiment,” or “an implementation” means that a particular feature, structure, or characteristic described in connection with an embodiment or implementation is included in at least one embodiment or implementation of the subject matter disclosed. Thus, the appearance of the phrases “in one embodiment” or “in an embodiment” or “in an implementation” in various places throughout the specification is not necessarily referring to the same embodiment or implementation. Further, the particular features, structures or characteristics may be combined in any suitable manner in one or more embodiments or implementations. Further, it is intended that embodiments and implementations of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values there between.

All of the functionalities described in connection with one embodiment or implementation are intended to be applicable to the additional embodiments and implementations described below except where expressly stated or where the feature or function is incompatible with the additional embodiments and implementations. For example, where a given feature or function is expressly described in connection with one embodiment or implementation but not expressly mentioned in connection with an alternative embodiment or implementation, it should be understood that that feature or function may be deployed, utilized or implemented in connection with the alternative embodiment or implementation unless the feature or function is incompatible with the alternative embodiment or implementation.

Aspects of the present disclosure are directed to systems and methods for generating context sensitive guidance (CSG) for medical diagnostics and the delivery of medical interventions by medical treatment and diagnostic devices. To alleviate the inherent challenges of an emergency trauma response, caregivers and rescuers can benefit from tools that provide potentially life-saving guidance for care and decision making. Such a system may monitor, integrate, and analyze information to enable caregivers to provide immediate and effective medical interventions that stabilize the patient and keep the patient alive long enough for the patient to receive longer term care, and possibly care for underlying conditions that may be the catalyst for trauma. For example, a person may be a trauma victim due to a car crash because they entered a diabetic coma or suffered a cardiac arrest behind the wheel of the car.

The context sensitive guidance (CSG) system, as described herein, provides care guidance in the context of presenting conditions of a patient often before an accurate diagnosis is likely or in some cases before such diagnosis is even possible. The CSG system collects objective physiological and other medical data for the patient and identifies therapeutic interventions tailored to alleviate physiological conditions indicated by the data. The care guidance identifies and enables implementation of interventions directed at the initial presenting conditions which in some cases pose an acute and immediate risk to the patient's life. Once the patient is stabilized, the CSG system may provide further guidance in identifying a diagnosis, or specific root cause, and identifying and implementing treatments based on the diagnosis. Additionally, or alternatively, the CSG system may repeatedly analyze physiologic data or other data about the patient to detect indicators of a likely resumption of an acute and possibly life-threatening medical condition. In some cases, a traumatic injury may present multiple medical conditions simultaneously. The CSG system described herein may determine relative priorities of the various conditions. For example, in the case of an extremity trauma without any other injury (e.g., a broken leg without any other injuries), the priority of care is to address the extremity trauma. However, in the case of a multi-system trauma (e.g., a broken leg along with head trauma), the CSG system may evaluate airway, breathing, and circulation along with the extremity trauma in the absence of bleeding or evaluate bleeding, airway, breathing, and circulation in the presence of bleeding along with the extremity trauma. As other examples, the CSG system may deprioritize care of a limb laceration if there are injuries to the core of the body, may deprioritize CPR if there is an active bleed, and may deprioritize stopping a nose or ear bleed if a cerebral spinal fluid leak is suspected in a head trauma event.

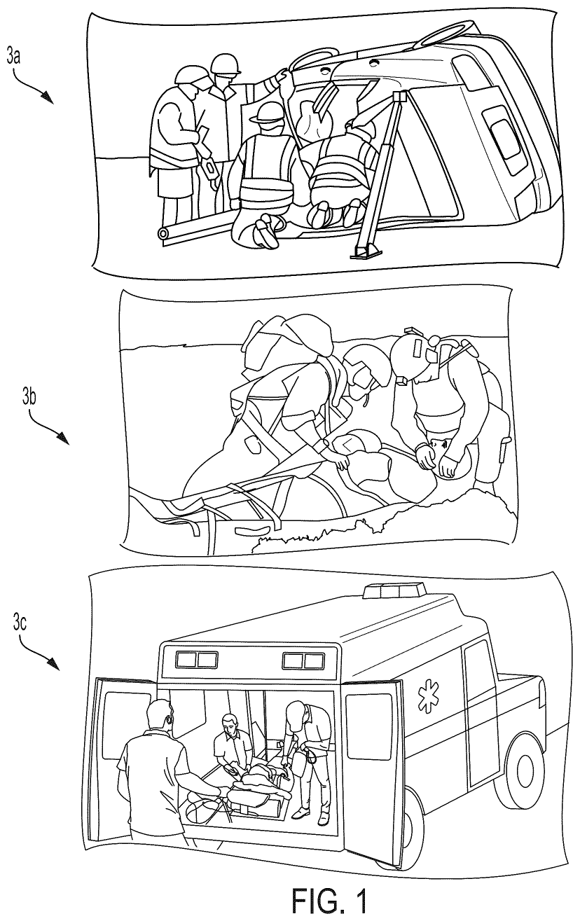

Referring to FIG. 1, examples of emergency care environments are shown. Such an environment may include a rescue scene, a military scene, or a transport vehicle scene, to name a few examples. The rescue scene 3a may be a scene of a car crash or another on-site trauma rescue scene such as a sporting event field, a blast site, a scene of a fall, etc. The military medical scene 3b may be on or near a battlefield, in a bay of a field hospital, in a military triage area, etc. The transport vehicle scene 3c may be inside an ambulance, as shown, or in a helicopter or other transport vehicle. The emergency care environment is not limited to the physical scenes shown and may also include a hospital or other medical care facility, a hospital emergency room, an urgent care clinic, a rural field hospital, an emergency medical tent, etc.

Regardless of the specific physical location, the emergency care environment may be a chaotic and crowded environment with many distractions in which acute critical care is necessary with a smaller choice of medical devices than might be available at a large hospital, for example. There may be multiple caregivers and/or multiple patients crowded into a small area. The medical skill and experience of the responding caregivers may vary (for example, the fire rescue personnel in the scene 3a may have less medical expertise than the ambulance crew in the scene 3c. Additionally, the medical skill and experience may vary within a team of caregivers. Thus, a context sensitive guidance (CSG) system, as described herein, that can adapt the guidance to available medical devices and/or caregiver skill level and/or available medical supply inventory, may improve patient outcomes. In one or more examples, provision of such a CSG system allows it to be installed in a variety of different locations with different provision of medical devices and inventory and/or operated by caregivers of differing skill levels, wherein the contextual data sources may provide for context relevant generation of the at least one action item and the at least one instruction. In some physical locations, a lack of network connectivity may prevent access to remotely located caregivers and computing systems, for example a cloud server. For example, a rural or military location, a parking garage, an interior location, or a moving vehicle may have little or no Internet or cellular network access or variable communication signal strength. Therefore, a CSG system, as described herein, that can adapt to provide guidance to with or without network connectivity may improve patient outcomes. Finally, first responders for trauma, or another medical emergency, cannot monitor a patient's condition for many hours, request lengthy lab or imaging procedures, or comb through a comprehensive medical history. Rather these first responders are tasked with making accurate split second and potentially lifesaving, decisions in the emergency environment.

The CSG system described herein provides many benefits, some of which are provided here as examples. This system provides adaptable and data-driven guidance that is sensitive to the particular context of the patient's physiologic status. This guidance includes an evaluation of which interventions to provide and how to provide those interventions. The CSG system may provide instructions to enable provision of interventions otherwise unavailable due to a lack of caregiver skill or an inability by the caregiver to quickly and accurately sort and analyze the vast quantity of information bombarding the caregiver during medical care. The CSG system may provide these instructions and guidance in a manner that may minimize or alleviate caregiver distraction and confusion. For example, the CSG system may provide user-selectable levels of detail. This may ensure that caregiver skill level and experience does not limit the provision of the determined care and, similarly, that unnecessary details do not slow or otherwise hamper that care. As another example, the CSG system may limit the physiologic information provided to the caregiver to actionable data in response to a detected degradation in a victim's medical state. As the caregiver and medical devices generate and collect patient data, the CSG system may monitor that information in the background of providing medical device data. In response to a detection of a degradation in the medical state of the patient, the CSG may move to the foreground. Thus, any monitoring UI may provide overall patient data in a first UI view and then transition to the CSG UI either automatically or by providing the caregiver with an option to obtain guidance. The CSG UI may provide for display of a subset of the patient data and/or physiological information selected by the CSG system based on said degradation in the medical state of the patient. Thus, the CSG system may tailor the data in the CSG UI to details of the health trajectory of the patient rather than providing a same set of data for every patient from which a caregiver would have to discern and determine which data items were more or less relevant, or perhaps irrelevant and distracting. In this sense, the CSG system provides data at the UI based on the medical state context of the particular patient. In trauma treatment situations, caregivers are often inundated with information, in part because of multiple physiologic systems typically injured as a result of trauma. The CSG system enables the caregiver to off-load the intake, the analysis, and the determination of the most efficient and effective response to a medical condition that may kill a patient in a matter of minutes. Further, the CSG system may relieve the caregiver of tracking their progress through a care sequence and of keeping track of possible missed or delayed steps. This system also determines, analyzes, identifies, monitors, and updates the parameters of provided interventions based on the physiologic data from the patient. In some cases, the physiologic data that is received, analyzed, and evaluated by the CSG system may enable the system to discern etiology and, thus, enable interventions and treatments directed at diagnosis.

In addition to guiding caregivers, the CSG system may instruct or control medical devices. For example, the CSG system may provide closed-loop control of various medical devices. The CSG system may inventory available devices and cross-reference that inventory to caregiver skill in order to tailor the use of medical devices to the victim's injury or presenting condition and to the caregiver's abilities. The CSG system may request information from the medical devices, send information to medical devices for storage, determine operational settings, etc.

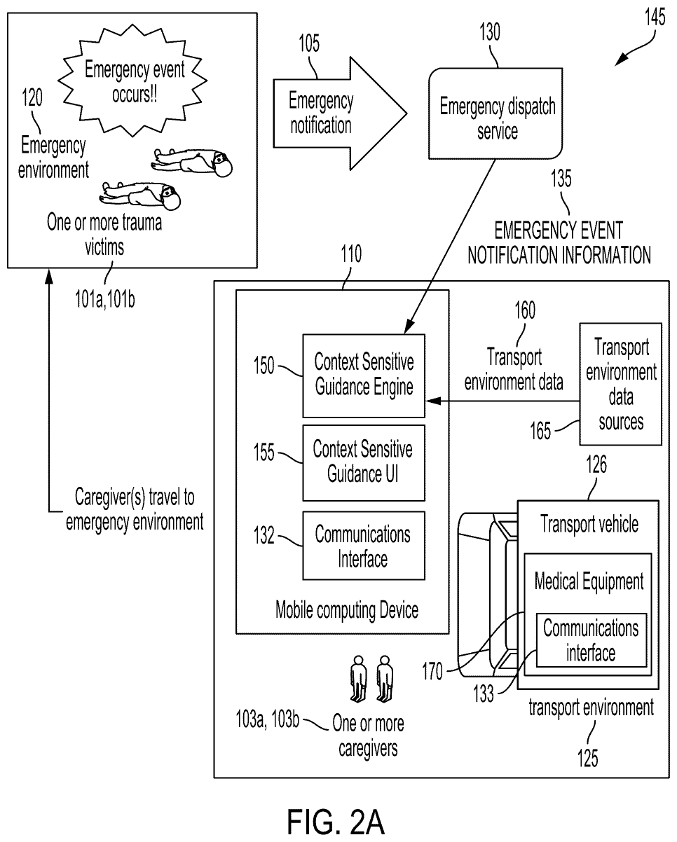

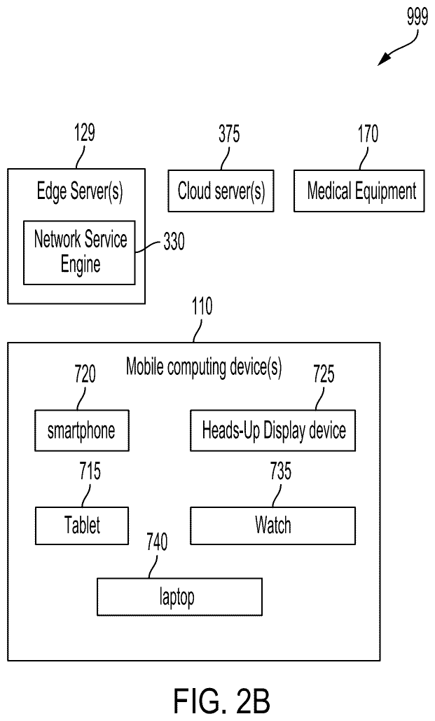

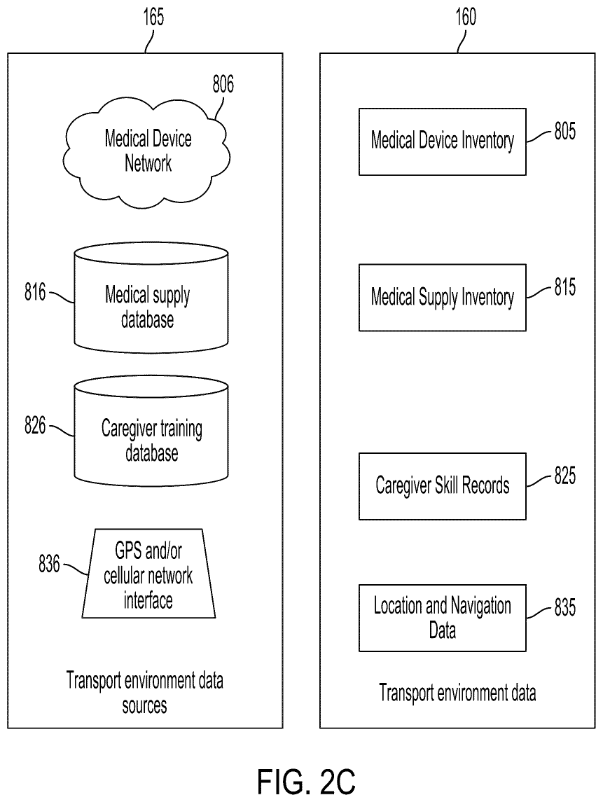

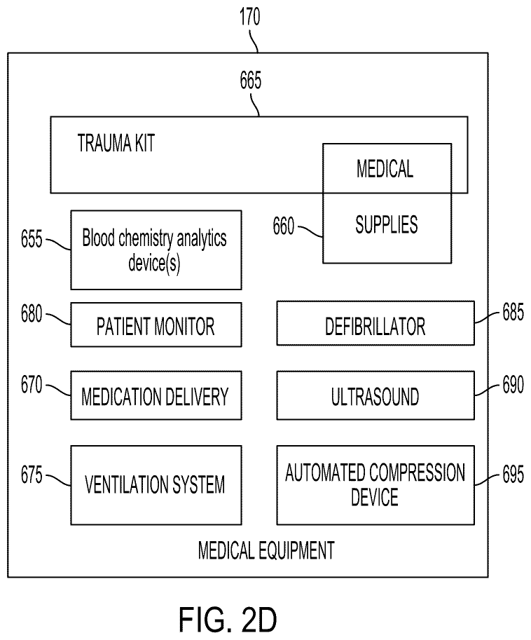

Referring to FIG. 2A, an example of a CSG system 145, which may be a trauma CSG system, deployed for treatment of patients or victims of an emergency medical event in an emergency environment is shown. The victims may be, but are not limited to, trauma victims. A quantity of each component in FIG. 2A is an example only and other quantities of each, or any, component could be used.

An emergency event may occur in an emergency environment 120. For example, the emergency environment may be one of the examples discussed in regard to FIG. 1. The emergency event may cause one or more victims 101a and 101b to suffer traumatic injuries. Traumatic injuries are severe physical injuries that occur suddenly and require immediate medical attention. These injuries are often the result of blunt, penetrating, and/or burn mechanisms and may result from a motor vehicle collision, a sports injury, a fall, a natural disaster, a blast, etc. Some common examples of traumatic injuries include but are not limited to traumatic brain injury, spinal cord injury, severed limbs, acoustic trauma, crush injury, concussion, fractures, cuts, and puncture wounds, collapsed lung, myocardial contusion, burns, electrical injuries, hypovolemic shock, hemorrhage, and hematoma.

In response to the emergency event, a victim or bystander may provide an emergency notification 105, for example via a 911 call, a 112 call, or a tactical operations center in a military setting. An emergency medical service (EMS) dispatch service 130 may receive the call and cause one or more first responders to be dispatched as caregivers (e.g., 103a and 103b) for the victims (e.g., 101a and 101b). The emergency notification 105 may include emergency event notification information 135. For example, the information 135 may indicate one or more of: who is injured (e.g., victim demographic information), where the injury took place (e.g., emergency environment location information), how the injury took place and/or what the injury is (e.g., a mechanism of injury (MOI)), the type of emergency event, the time of the emergency event, etc. The emergency event notification information 135 may be generated by the emergency dispatch service 130.

The dispatched first responders 103a and 103b may travel to the emergency scene in a transport vehicle 126 such as an ambulance, fire engine, police car, helicopter, etc. Computing resources 999 associated with and/or available to the caregivers and/or the transport vehicle 126 may include a mobile computing device 110. The mobile computing device 110 may include CSG engine 150. The CSG engine 150 may include a CSG engine 150 and non-trauma CSG engines (e.g., respiratory distress CSG engine, cardiac arrest CSG engine, etc.). The CSG engine 150 may provide a CSG UI 155, In an example, the CSG UI 155 may be a trauma CSG UI with features specifically directed at CSG for trauma. A patient presenting with trauma may also present with other disease states in parallel with trauma and/or caused by the trauma and the CSG UI 155 may provide guidance for treatments and interventions for the full scope of patient conditions. The CSG engine 150 includes a CSG engine 150 and non-trauma CSG engines. For example, the non-trauma CSG engines may include a respiratory distress engine, a cardiac engine, a sepsis engine, etc. Furthermore, the CSG UI 155 generated by the CSG engine 150 may include a trauma CSG UI, a non-trauma CSG UI (e.g., a respiratory distress UI, a cardiac UI, a sepsis UI, etc.) or a UI that is a combination thereof (e.g., a combination UI providing guidance for treatment of trauma along with other non-trauma conditions, treatments, or interventions, for example a combination of one or more of trauma, respiratory distress, cardiac, ultrasound sepsis, etc.). As discussed in further detail below with regard to FIG. 2B, the CSG engine 150 may include the computing resources 999. The computing resources 999 may include the mobile computing device 110, an edge server 129, the medical equipment 170, or combinations thereof.

Upon dispatch and on route to the emergency environment 120 or at other times, the CSG engine 150 can initiate context sensitive guidance (CSG) to prepare for the provision of medical care to the victim(s) prior to arrival at the emergency environment 120. The CSG engine 150 may determine this initial CSG based on data about the victim(s) available prior to arrival at the emergency scene 120. This data may include the emergency event notification information 135 and/or transport environment data 160. As discussed in further detail below with regard to FIG. 2C, the transport environment data 160 may include, for example, a medical device inventory 805, a medical supply inventory 815, a caregiver skill record 825, and/or location and navigation data 835. One or more transport environment data sources 165 may provide the transport environment data 160 to the CSG engine 150.

With the emergency event notification information 135 and the transport environment data 160, the CSG engine 150 may select at least one action item for the EMS crew (e.g., caregivers 103a, 103b) and/or the medical devices prior to the arrival of the EMS crew at the emergency scene 120. This may improve the care provided to the victim by optimizing a response prior to arrival and enabling immediate deployment of that response upon arrival.

For example, the CSG engine 150 may generate a recommendation of medical equipment to bring to the emergency scene based on the notification information. The CSG engine 150 may evaluate an inventory of the medical equipment 170 and determine if the inventory includes the recommended medical equipment. If the inventory includes the recommended medical equipment, then the CSG engine 150 may provide instructions on preparing this equipment for deployment in the emergency environment 120 and/or provide instruction for use. If the inventory does not include the recommended medical equipment, then the CSG engine 150 may adjust the recommendation based on what is available and/or may provide a request to an emergency agency to deliver the recommended equipment to the emergency environment 120. As discussed in further detail below with regard to FIG. 2D, the medical equipment 170 in the inventory may include medical devices and medical supplies. For example, the medical equipment 170 may include one or more of a defibrillator 685, a patient monitor 680, a trauma kit 665, a ventilation system 675, an automated compression device 695, an ultrasound device 690, a medication delivery device 670, blood chemistry analytics device(s) 655, medical supplies 660, or combinations thereof. For example, the CSG engine 150 may look for a blood glucose kit on the inventory to determine if an evaluation of a head trauma victim for hypoglycemia will be possible on scene.

As another example, based on the MOI, the CSG engine may recommend protocol training segments to review prior to arrival such as bleeding, spine stabilization, splinting, airway protection (e.g., manual ventilation or intubation), and CPR compressions. Based on responders' level of training, the CSG engine 150 may suggest and/or assign roles to various caregivers, wherein said roles may define which of a plurality of medical interventions and/or assessments that are determined likely to be required are to be performed by whom, such as, for example, stabilize cervical spine, address major bleeding, perform initial patient assessment, provide CPR compression, provide airway protection, etc. In an implementation, based on the MOI, the CSG engine 150 may provide a guidance review based on interventions likely to be required for the victim. In an implementation, the caregivers 103a and 103b may be second responders and the CSG engine 150 may receive information from first responders already on scene. For example, the first responders may request additional resources such as a defibrillator, cardiac compression device, specialized transport devices, etc. The CSG engine 150 may also evaluate the MOI to determine a likelihood that the victim(s) will require spinal stabilization. For example, a high-speed vehicle collision or a collision between an automobile and a bicyclist or pedestrian may indicate a high likelihood of necessary spinal stabilization. The CSG engine 150 may provide caregiver instructions to prepare for spinal stabilization, by preparing equipment, reviewing protocol instructions, etc.

Referring to FIG. 2B, examples of computing resources are shown. A quantity of each component in FIG. 2B is an example only and other quantities of each, or any, component could be used. The computing resources 999 may include one or more mobile devices 110, an edge server 129, the medical equipment 170, a cloud server 375, or combinations thereof.

The mobile device 110 is a caregiver interface device as it enables the caregiver to interact with the CSG engine 150. For example, the mobile device 110 enables the caregiver to provide input to the CSG engine 150 such as spoken input or input entered at a touchscreen. The mobile device 110 enables the caregiver to receive output from the CSG engine 150 such as prompts, instructions, reminders, etc. as visible, audible, and/or tactile output. In various implementations, the mobile device 110 may include one or more of a tablet 715, a smartphone 720, a heads-up display device 725, a watch 735 and/or a laptop 740. In various implementations, the heads-up display device 725 may be one or more of a virtual reality device and an augmented reality device.

One or more items of medical equipment 170, for example a medical device such as but not limited to a defibrillator 685, a ventilation system 675, or a patient monitor 680, may provide computing resources. In this capacity, the medical equipment 170 may implement all or a portion of the CSG engine 150.

In an implementation, the computing resources may include an edge server 129. In an implementation, the edge server 129 may be a component of the mobile device 110 and/or a medical device, such as a defibrillator, included in the medical equipment 170. The edge server 129 may enable the CSG engine 150 to run without any cloud connection by providing more substantial processing and computational resources than those available on the mobile devices 110 and/or the medical equipment 170. The edge server 129 may monitor for communication connections with a cloud server 375 and may connect and synchronize with this server when the communication connection is available. The edge server 129 may also provide some security functions as a computing entity that sits in between the mobile devices 110 and the cloud server 375.

The cloud server 375 may be configured to provide and execute artificial intelligence (AI) models to analyze data from the transport and emergency environments. In an implementation, the cloud server 375 may host the CSG engine 150 and service a CSG application at a local device (e.g., the mobile device 110, the medical equipment 170 and/or the edge server 129). The CSG application may be configured to run locally in the absence of a network connection to the cloud server 375. For example, the mobile device 110 may locally implement the CSG engine 150 without connectivity to the cloud computing environment. The emergency environment may be a rural area, an underground area, like a parking garage, an interior space, an urban canyon, a military battlefield, etc. In the military application, connectivity to a network may endanger both caregivers and victims as it may enable tracking of their location by opposition forces.

The edge server 129 may include a network service engine 330 to monitor a connectivity status between the edge server 129 and the cloud server 375. The network service engine 330 functions as a service worker configured to monitor and recognize a network connectivity status between the local computing devices (e.g., edge server 129, the mobile devices 110, and/or the medical equipment 170) and the cloud server(s) 375. The network connectivity status may be a connected status or an unconnected status. When the network connection is active, the network service engine 330 sends out application program interface (API) calls in order to invoke the cloud hosted CSG engine. When the network connection is inactive, the network service engine 330 stores API call records for activation when the network connection resumes.

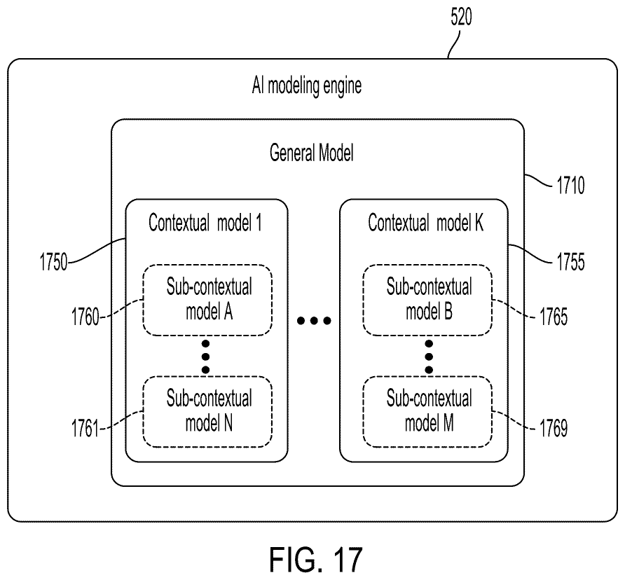

In an implementation, the CSG engine 150 may be provisioned with streamlined AI models that enable the engine 150 to function effectively at the mobile device 110 without cloud connectivity. These streamlined models are discussed further below in regard to FIG. 17. However, the predictive capability of more complex models that require the processing capability of the cloud server 375 may enable model outputs with higher associated confidence levels. In an implementation, the CSG engine 150 may utilize machine learning model(s) that are locally stored at the mobile device 110 in the unconnected status of the mobile device 110 and may utilize machine learning model(s) applied by the remote cloud server 375 in the connected status. In an implementation, the CSG engine 150 may utilize a combination of the locally implemented and remotely implemented model(s) in the connected status.

The network service engine 330 may monitor and track requests from the CSG engine 150 to send and/or receive information from the cloud server 375. If connectivity with the cloud server 375 is disrupted or if the connectivity is absent at the time of the request, the network service engine 330 may queue the tracked requests and then provide the data exchange when the connection is available or reestablished. In the connected status the CSG engine 150 may access additional information (e.g., medical records, telemedicine provider) along with additional and/or more complex machine learning models. One or both of these may increase the confidence level of outputs for the CSG engine 150 and thus may change or modify output from the engine 150 (e.g., a selected action item for medical care and/or instructions for the caregiver and/or medical device). This change or modification may occur in response to a transition from the unconnected status to the connected status. In other words, once information becomes available to the CSG engine 150 from the cloud server 375, the CSG engine 150 may recognize this availability (e.g., as controlled in an automated fashion by the network service engine 330) and respond accordingly.

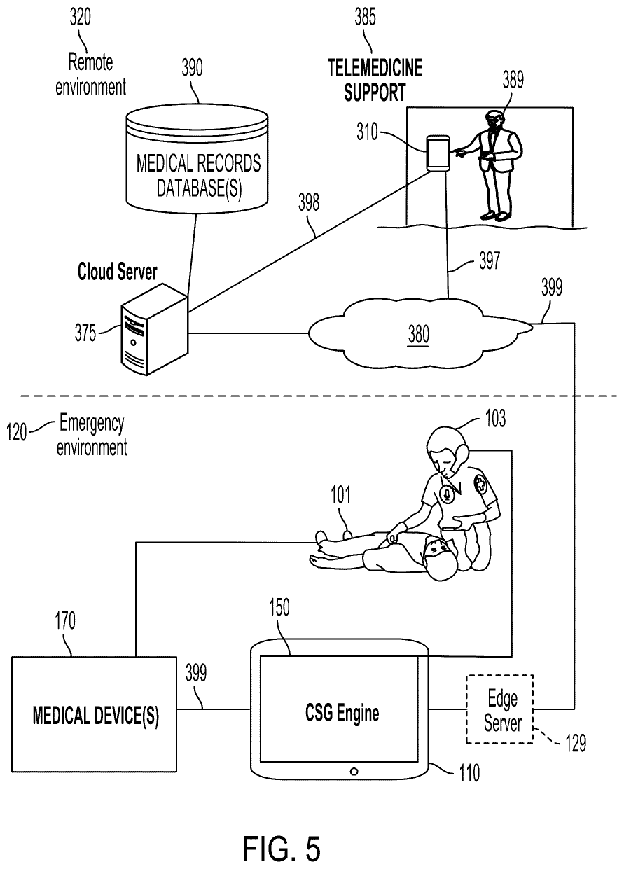

Each device shown in FIG. 2B may be communicatively coupled to one or more of the other devices. The CSG engine 150 may be disposed at one of the devices shown in FIG. 2B or may be a distributed computing system disposed at two or more of the devices shown in FIG. 2B. Further, one or more of the devices shown in FIG. 2B may be communicatively coupled to one or more computing devices of the remote environment 320 as shown in FIG. 5.

Referring to FIG. 2C, examples of transport environment data and transport environment data sources are shown. A quantity of each component in FIG. 2C is an example only and other quantities of each, or any, component could be used. Thus, one or more of the elements shown may be provided.

The transport environment data 160 may include the medical device inventory 805, the medical supply inventory 815, the caregiver skill record 825, and location and navigation data 835. The transport environment data sources 165 may include a medical device network 806, a medical supply database 816, a caregiver training database 826, and a global positioning system (GPS) and/or cellular network interface 836. One or more of the computing resources 999 may include the databases 816 and 826. Additionally, one or more of the edge server 129, the mobile devices 110, and the medical equipment 170 may include the GPS and/or cellular network interface 836.

In an implementation, one of more of the medical devices included in the medical equipment 170 may be communicatively coupled to one another and/or to the mobile device(s) 110 to form the medical device network 806. In an implementation, the edge server 129 may orchestrate communications between the elements of the medical device network. The medical device network 806 may be a Wi-Fi network, a short-range wireless communication network or near field communication (NFC) network, local area network (LAN), wide area network (WAN), the Internet, or a cellular communication network. In some implementations, the medical equipment 170 may function as a wireless access point to provide a direct wireless connection with the mobile device 110. The medical device network 806 enables data to be securely and accurately shared between two or more devices in the transport environment 125 and/or in the emergency environment 120.

In one or more examples, the CSG engine 150 may receive a medical device inventory 805 via a communicative coupling with the medical device network 806. In order to collect the medical device inventory 805 and/or the medical supply inventory 815, in an implementation, the CSG engine 150 may poll the transport environment 125, the emergency environment 120, and/or a particular patient care environment 320a or 320b in order to inventory the medical equipment 170. For example, communications interfaces (e.g., the communications interface 132 and the communications interface 133 shown in FIG. 2A) may communicatively couple the mobile device 110 with one or more medical devices or items of medical equipment in the medical equipment 170 individually or via the medical device network 806. The communications interface 132 may identify the devices and/or equipment based on information exchanged with individual medical devices and/or with the medical device network 806. For example, the communications interface 132 may identify one or more of a type of a coupled device (e.g., the types of devices shown in FIG. 2D), a capability of a coupled device (e.g., a therapy delivery capability, a monitoring capability, an imaging capability, a sensor attachment capability, a mode of operation, etc.), and/or a model number.

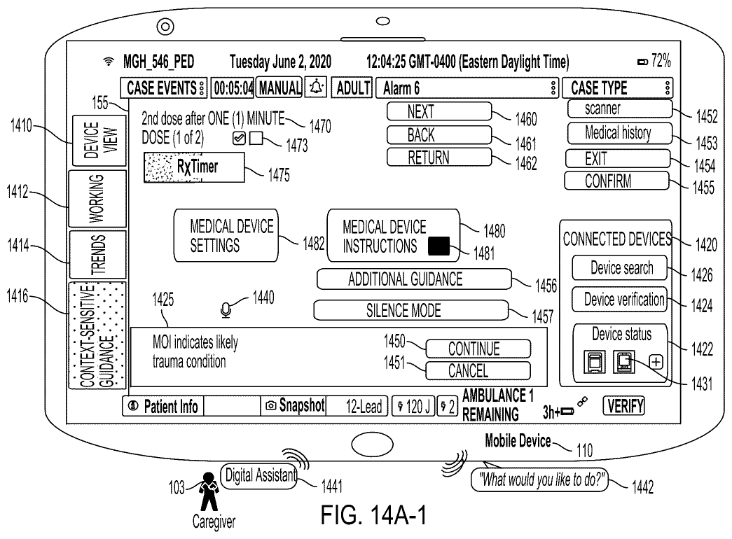

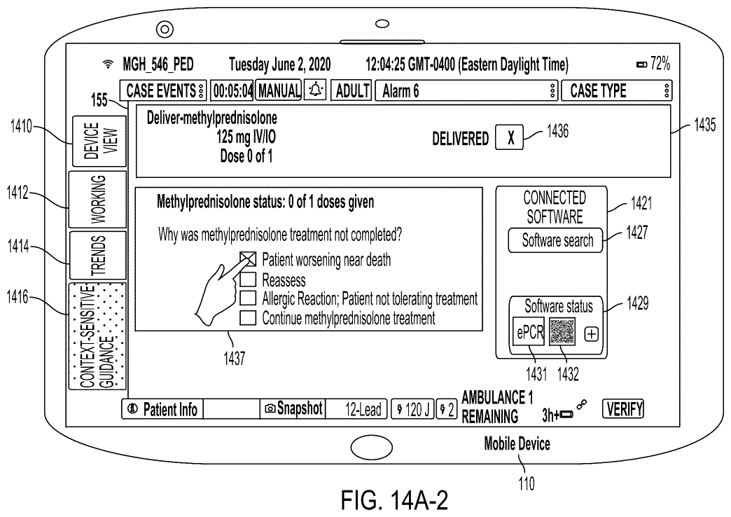

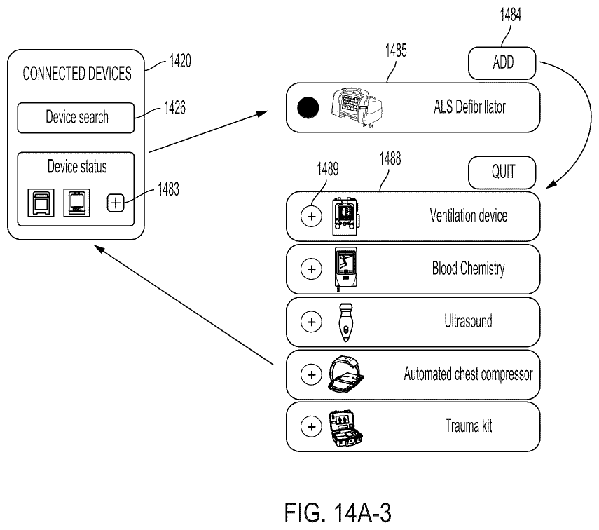

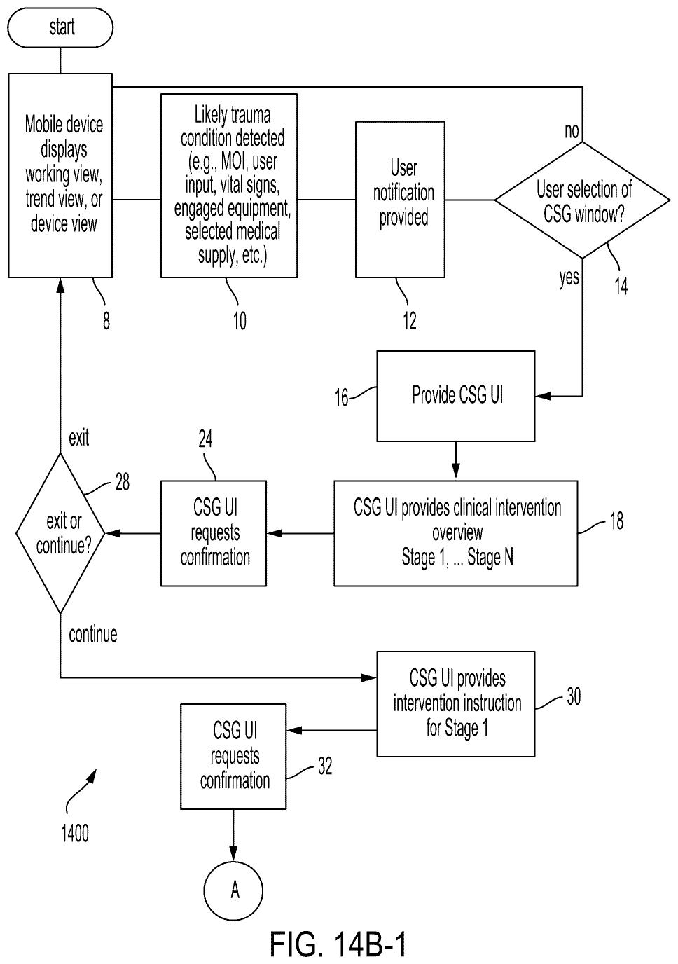

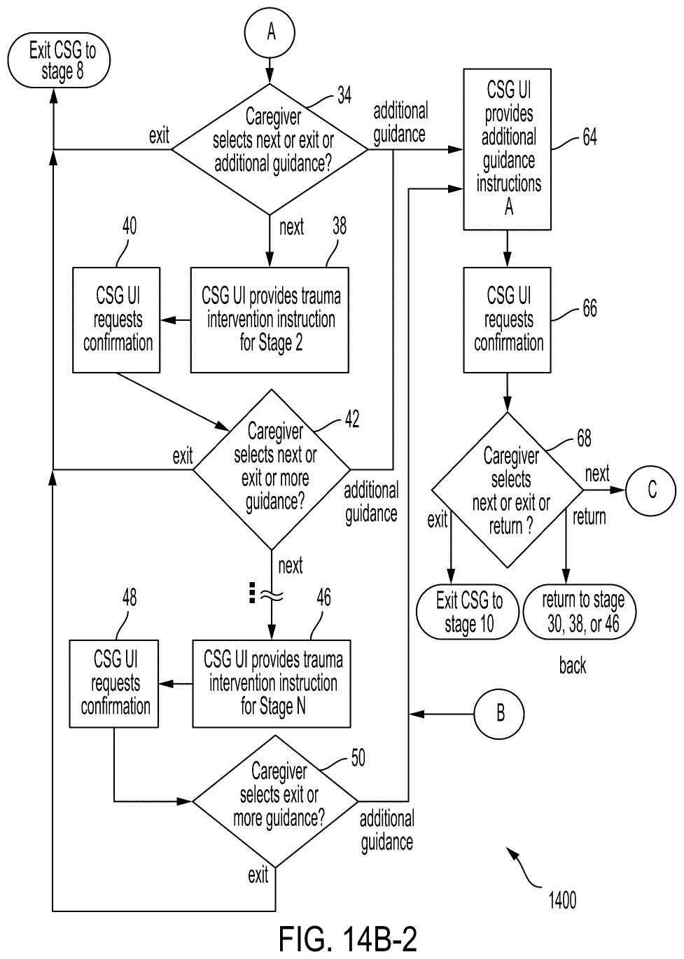

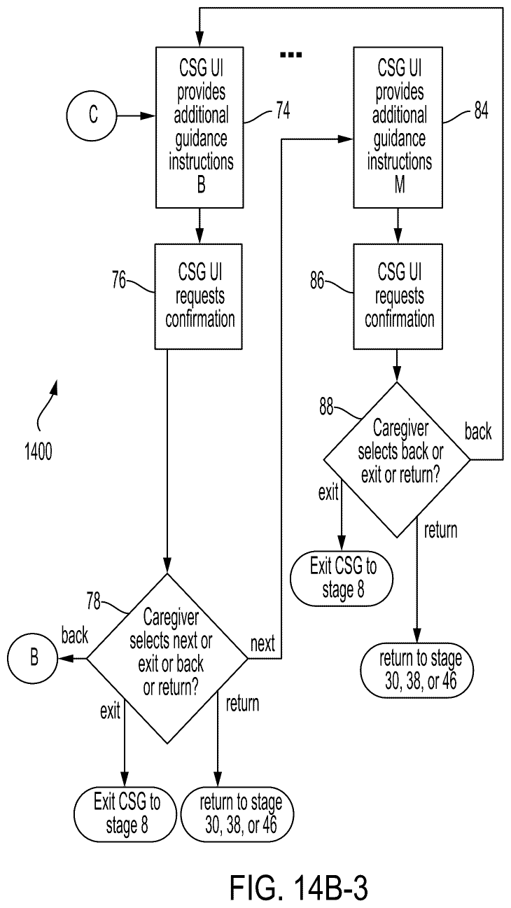

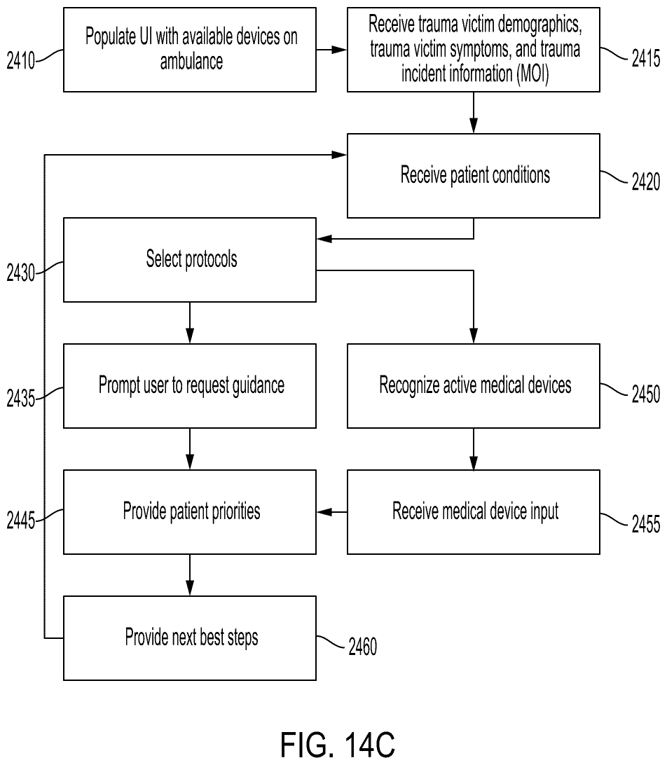

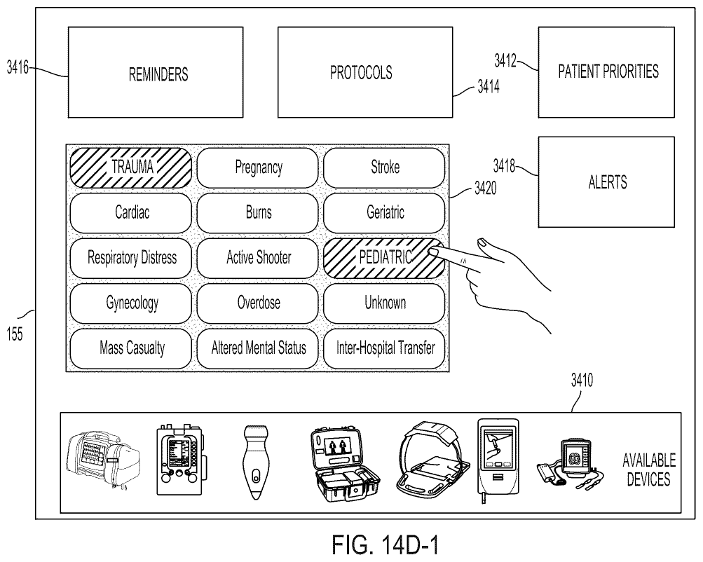

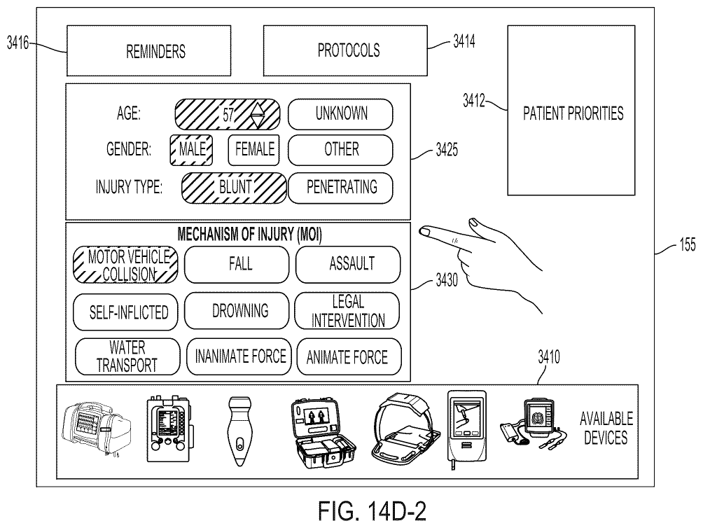

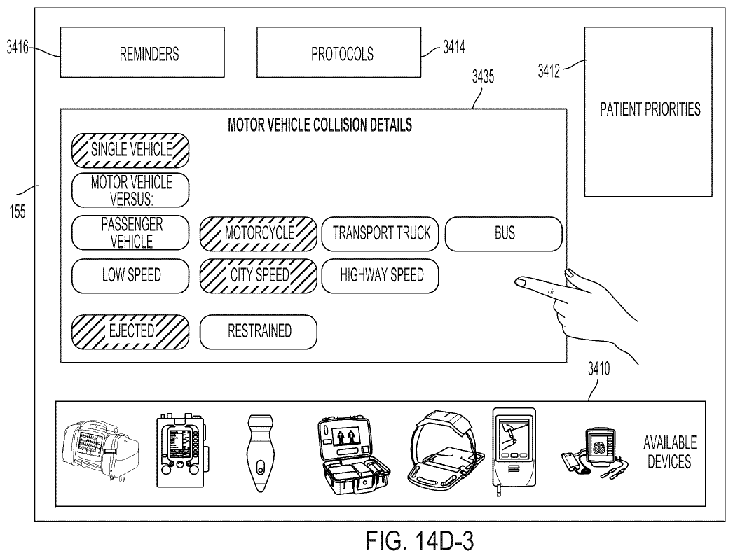

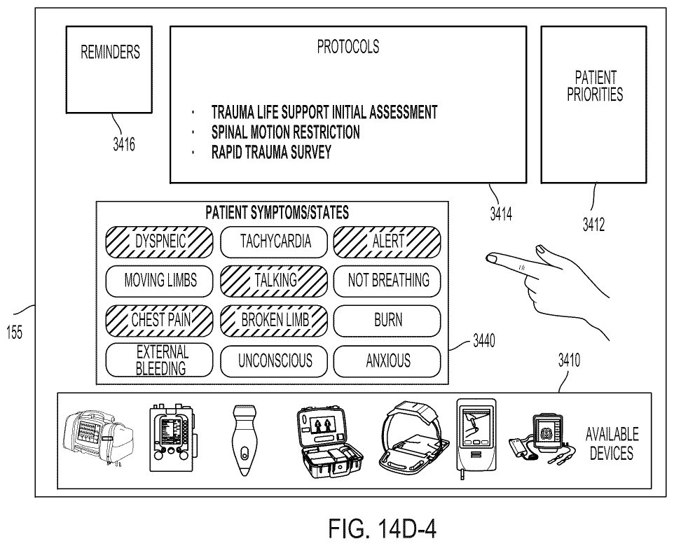

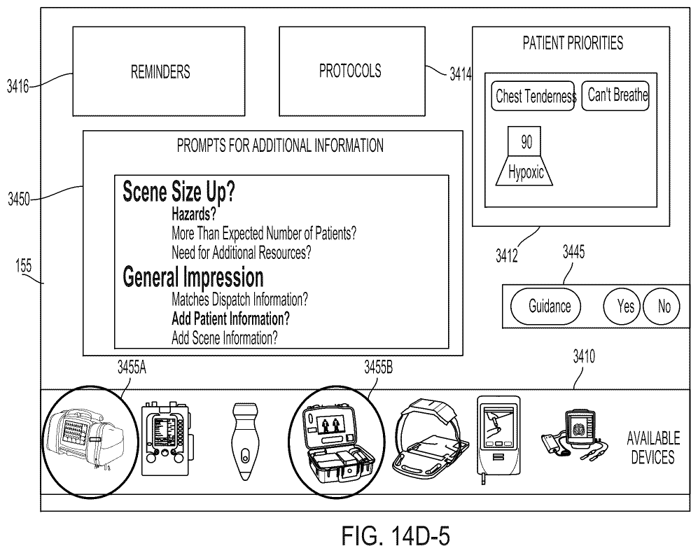

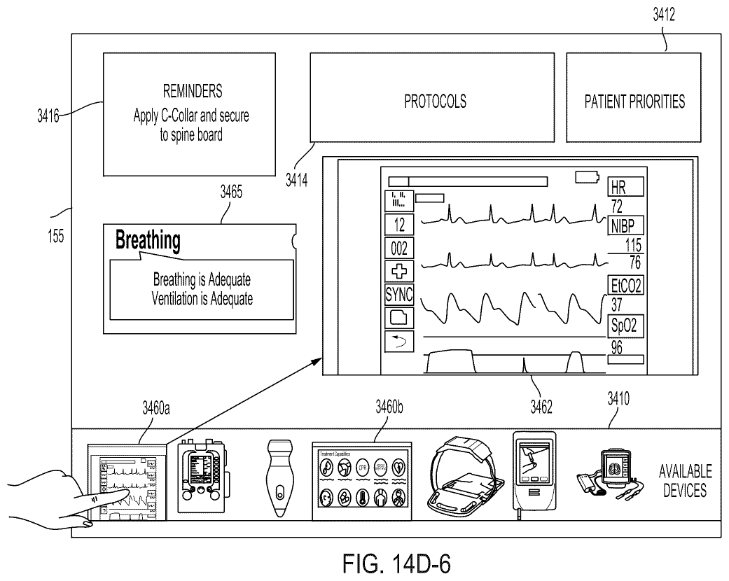

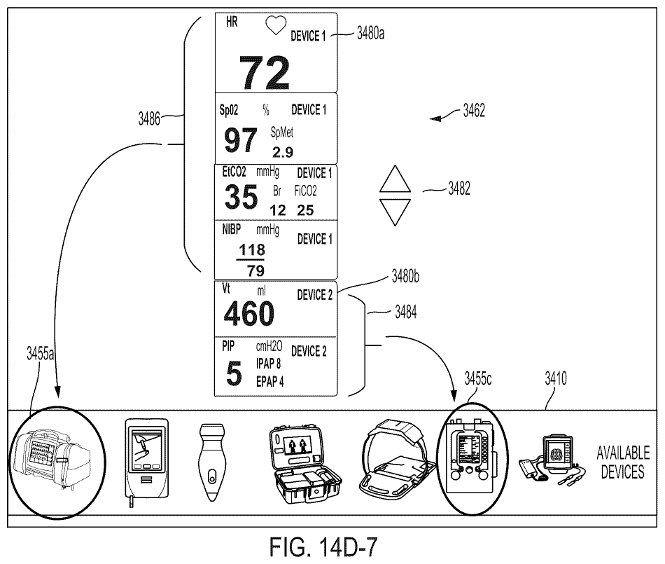

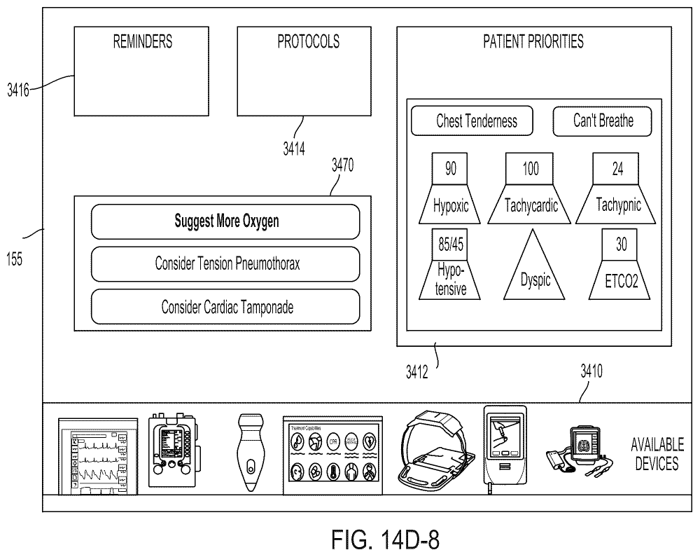

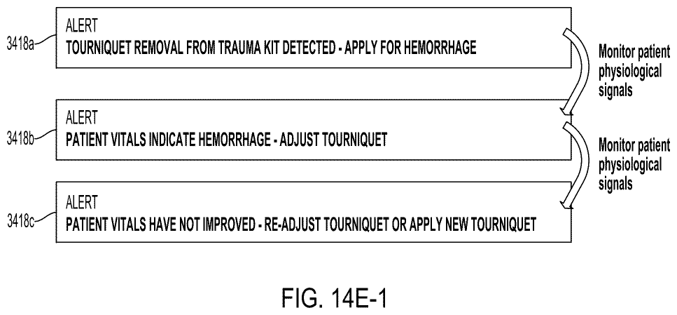

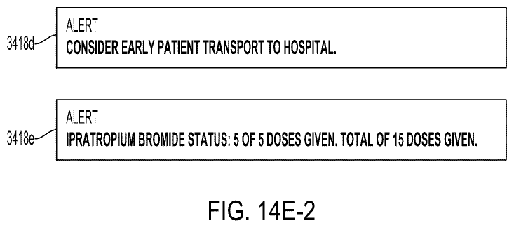

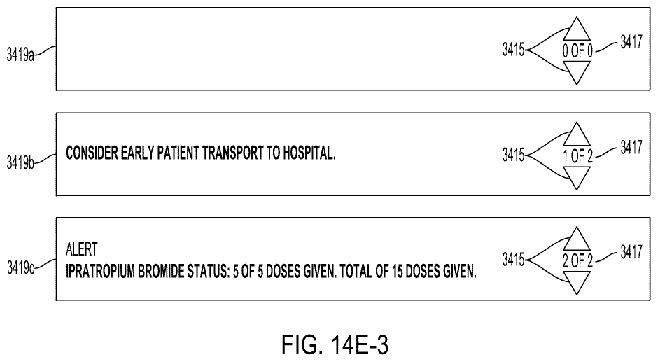

In an implementation, the CSG engine may provide a CSG UI, for example a CSG UI 155, that includes a contextual data source window such as, for example, a connected devices window 1420 (e.g., as shown in FIG. 14A-1) that displays the various connected devices and equipment. In an implementation, the mobile device 110 may couple with a first or initial medical device, for example, on route to the emergency environment or at the outset of patient care. Subsequently, other devices (e.g., at least one additional medical device) may establish communications with the mobile device 110 and/or join the network 806. The communications interface 132 may identify the subsequent devices based on the communicative coupling. The CSG engine 150 may update the connected devices window 1420 to include the identification of these subsequent devices as they are added to the system.

The connected devices window may include a device status window 1422, a device search control 1426, and/or a device verification control 1424. The device status window 1422 may display one or more of an amount of battery life, connection status, and a unique name for each of the one or more items of medical equipment 170 that is communicatively coupled to the mobile device 110. In an implementation, the device status window may display a medical equipment icon 1430 for a connected device with a different color (or other form of graphical distinction) than the icon for an unconnected device. The paired device verification input selector 1424 may enable the mobile device 110 to cause an indicator to flash (or provide another indication) at the one or more medical equipment 170 to confirm that the mobile device 110 is connected to the correct medical device at time of use. In some examples where multiple medical events are occurring at the same time, such as in a trauma unit of a hospital or on a scene of a mass casualty or collision, there may be multiple rescue teams operating multiple medical treatment devices within close proximity of one another. In such situations, it may be easy to mix up mobile devices that are paired to respective medical treatment devices. Therefore, in some implementations, when the verification input selector 1424 is selected, the mobile device 110 may generate an instruction signal that causes the connected medical equipment 170 to generate an indication of being paired with the mobile device 110. In some implementations, upon receiving a verification signal from the mobile device 110, the medical equipment 170 may output a visual and/or audible indication of being connected to the mobile device 110. In some examples, the indication can be a flashing light and/or a tonal sound pulse. The device search control 1426 may enable a display at the mobile device 110 to display wireless communication links that are available for the mobile device 110 to connect to the medical equipment 170. One or more of these links may be pre-configured.

In an implementation, a CSG UI, for example, the CSG UI 155, may include a that includes a contextual data source window such as, for example, a connected software window 1421 (e.g., as shown in FIG. 14A-2). The connected software window 1421 may display the various software and/or applications in communication with the CSG engine 150 and/or the mobile device 110. The CSG engine 150 may couple with other software or firmware on the same device as the CSG engine 150 and/or on another device via a connection between In an implementation, the mobile device 110 providing the CSG engine 150 may couple with one or more other computing devices or one or more medical devices in order to enable an exchange of data between another software application and the CSG engine 150. The CSG engine 150 may update the connected software window 1421 to include the identification of software as it is connected or disconnected.

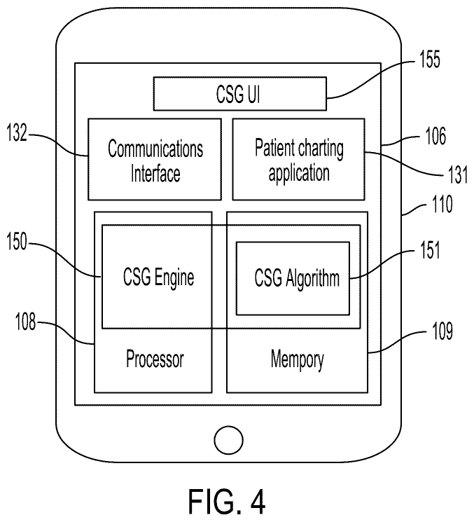

The connected software window may include a software status window 1429, a software search control 1427, and/or a software verification control 1423. In an implementation, the device status window 1429 may display a software identification icon 1431 for connected software with a different color than the icon for software that may be commonly available but currently unconnected. In an implementation, the CSG engine may generate a connections indicator at the UI of connected software and/or at a device providing the connected software. The software search control 1427 may enable a display at the mobile device 110 to display wireless communication links that are available for the mobile device 110 to connect to the various software. One or more of these links may be pre-configured. In an implementation, the connected software may be a charting application such as the patient charting application 131 (e.g., as shown for example in FIG. 4).

The CSG UI 155 may include a code generator 1432 that enables the CSG engine to generate a bar code or a QR code with encrypted information that enables other computing and/or medical devices to receive medication, patient, or emergency incident information and/or establish a communication channel with other software applications, computing devices, and/or medical devices. For example, the code generator 1432 may generate a bar code for medication information and then a computing device on scene other than the mobile device providing the code generator 1432 may scan the bar code and receive the medication information for recordation, display, and/or transmission. As another example, the code generator 1432 may generate a QR code with patient and/or event information that enables another computing device on scene other than the mobile device providing the code generator 1432 to scan the code and access patient records and/or other event information. For example, the QR code may enable a communication connection between the CSG engine and a patient charting application. As another example, the QR code may enable multiple computing devices to access the CSG engine 150 and CSG UI 155 for a same emergency incident. This may enable multiple caregivers for a victim or victims to share information on an ongoing basis or in real-time during the emergency response. As a further example, the code generator 1432 may generate a QR code with device connectivity information (e.g., information about the various connected computing and/or medical devices which, for example, may include one or more of the devices shown in the available devices window 3410). For example, the generated QR code may provide the device type, the serial number for the device, and the internet protocol address for the device. A computing device other than that providing the QR code generator and/or a medical device may scan the QR code to enable a communicative coupling with the device represented by the generated QR code.

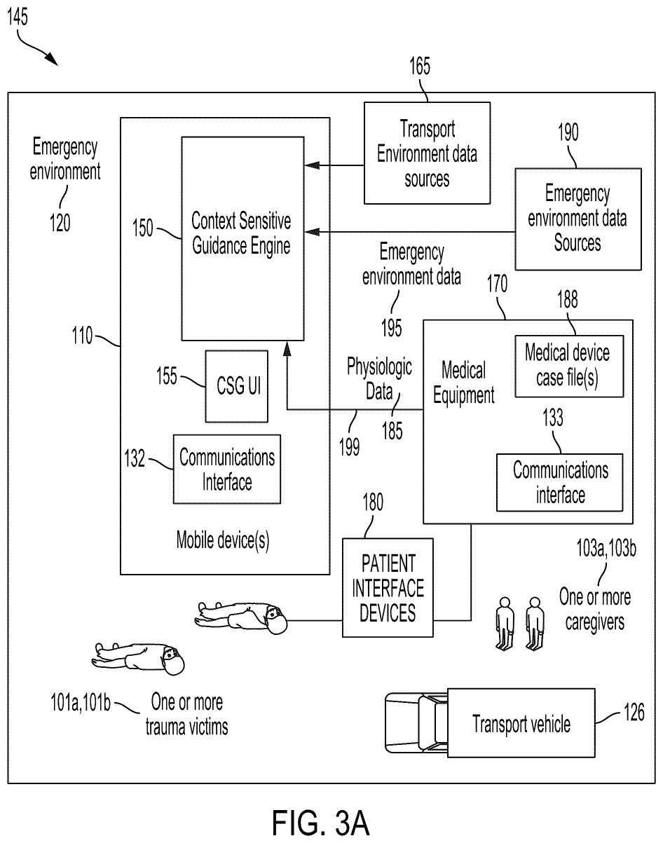

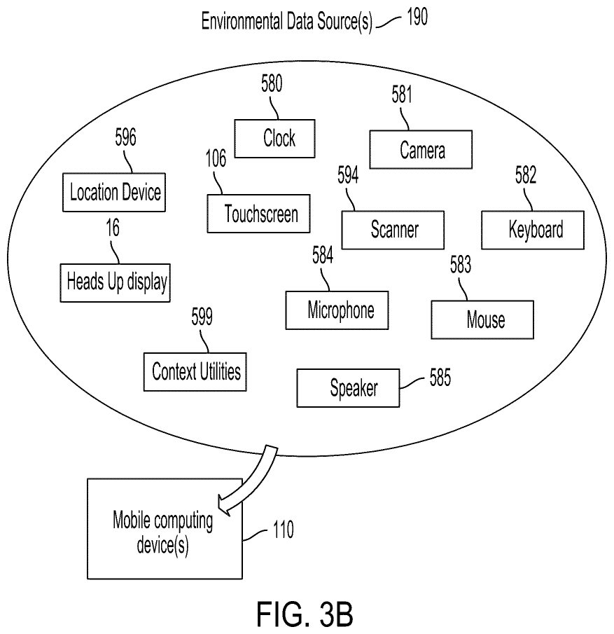

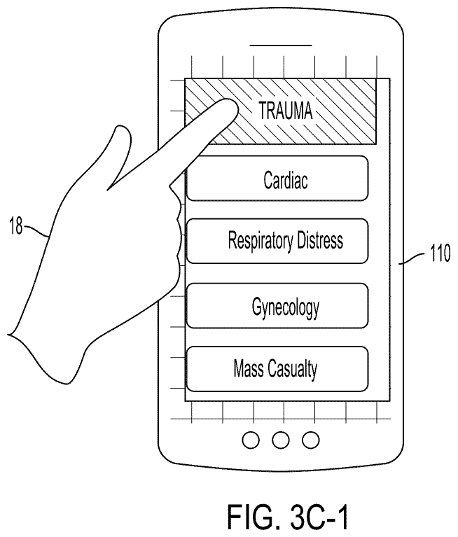

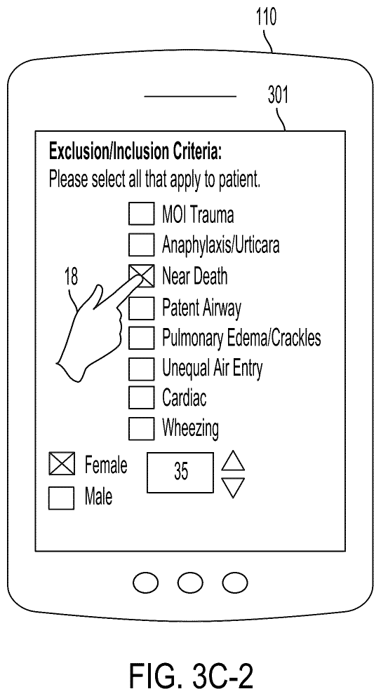

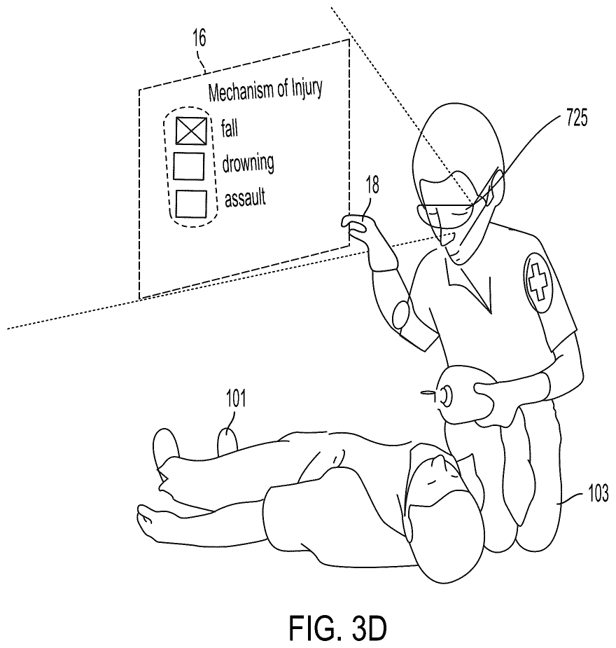

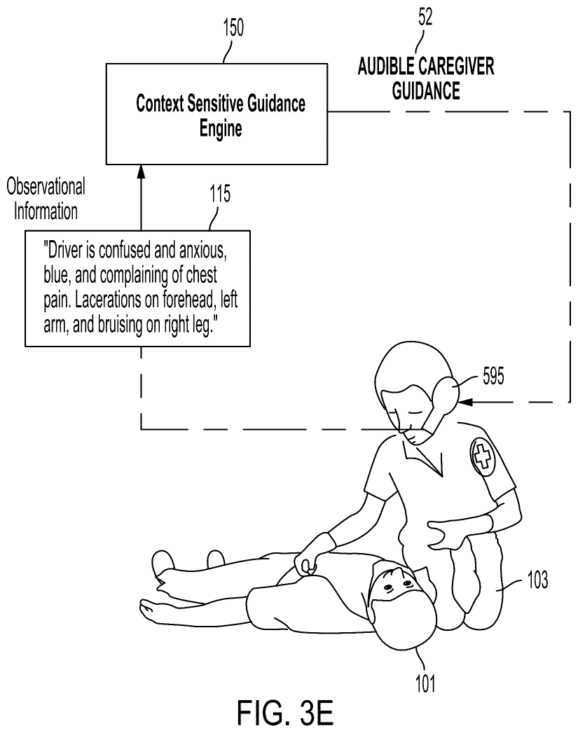

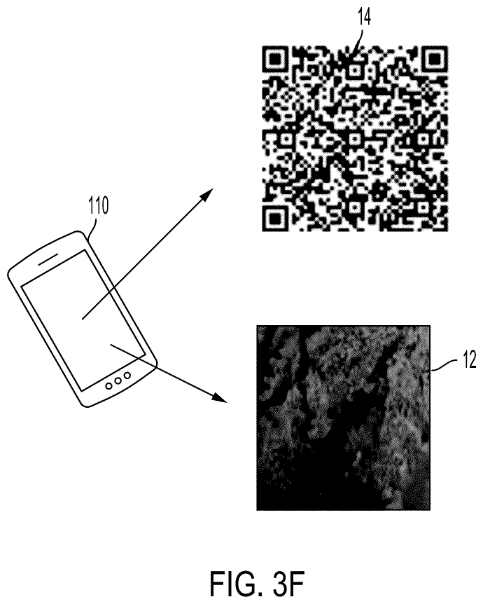

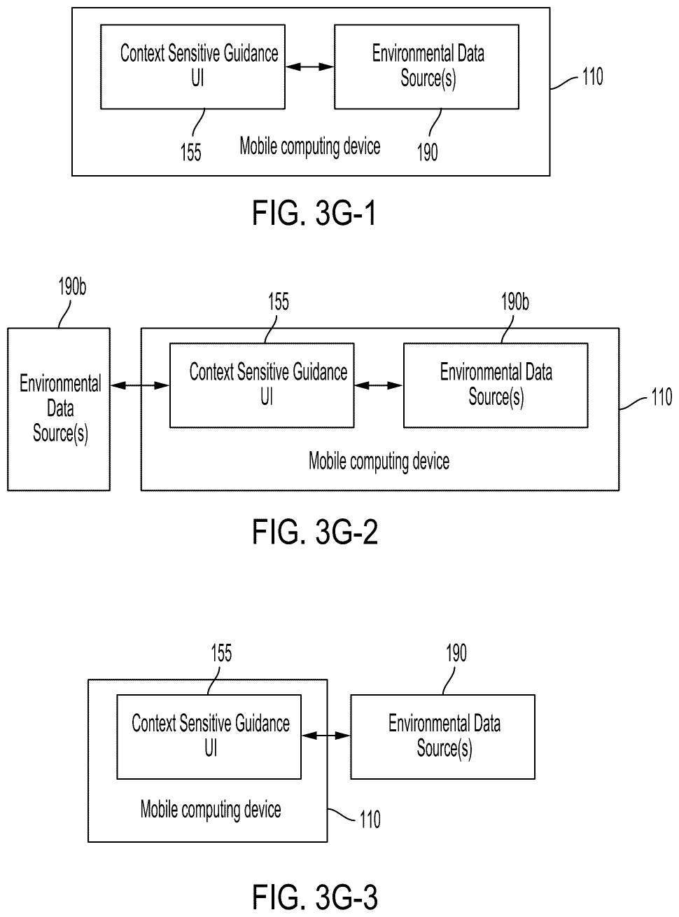

As shown for example in FIG. 14A-3, in an implementation, the connected devices window 1420 may provide manual device connection controls. For example, selection of the device search control 1426 by the caregiver, or another control provided by the connected devices window 1420, may cause the CSG UI 155 to show a connected devices menu 1485 that shows the currently connected devices and provides an add control 1484. Selection of the add control 1484 may cause the CSG UI 155 to show a menu of available devices 1488. Each item in the menu 1488 may include a selection control 1489. The caregiver may manually select one or more of the available medical devices in the menu 1488. The menu 1488 may include one or more of the medical equipment 170, medical supplies 660, and the computing resources 999 (which may include one or more mobile computing devices 110 other than the mobile computing device providing the menu 1488). Once the caregiver has completed their selection, the CSG engine 150 will communicatively couple with the selected devices. In an implementation, CSG UI 155 may display a summary of the connected devices in the device status window 1422. In an implementation, the CSG UI 155 may display device status window 1422 with an add control 1483 in lieu of the add control 1484 and the menu 1485. Selection of the add control 1483 may generate the menu 1488 to enable manual requests for communication channels.

In certain embodiments, the wireless communications interface 133 of a respective medical device included in the medical equipment 170 can be configured to detect that a respective mobile device 110 is within communication range and in response, initiate one or more actions to connect to the mobile device 110 via the wireless communication link 199. In some implementations, a mobile device 110 that is pairable with the medical equipment 170 can be preconfigured as a companion device to automatically connect to the medical equipment 170 via the wireless communication link 199 when within communication range, without having to discriminate between other devices that happen to be within range and/or negotiate a wireless communication connection. Further, rather than requiring a user to potentially spend significant amounts of time in manually configuring the system of each companion mobile device to connect to the medical equipment 170 or accessing a screen to view and then select from possible device connections, mobile device(s) located at the emergency scene may be pre-configured to dynamically join and/or leave the secure network 806 or pair with the medical equipment 170, for example, automatically and/or with one or more simple actions (e.g., switch actuation, pressing a button, near field communication connection, radio frequency, location/proximity recognition, gestural code, tap/bump recognition, motion-activated, sound/vibration, voice command/recognition, amongst others) and/or merely by being in close physical proximity to one another such as by a Bluetooth proximity connection. For example, the mobile device 110 may provide user selectable list of pre-configured wireless communication links that are available for the mobile device to connect to the medical equipment 170. The user can also view other available networks that have not been pre-configured for connection. In some examples, the mobile device may be pre-configured for pairing to other medical treatment devices, and those preconfigured networks can also be displayed at the mobile device 110. The connected devices window 1420 (e.g., as shown in FIG. 14A-1) may display the connected devices.

As part of the communicative coupling of the medical equipment 170 to the mobile device 110, the communications interface 132 may authenticate the medical equipment 170 and establish the communicative coupling in response to and based on the authentication. The CSG engine 150 and/or the communications interface 132 may enable data encryption of some or all of the data exchanged between the mobile device 110 and the medical equipment 170. In an implementation, the data encryption may separately shield patient data, in particular any patient identifying data (PID), to prevent or limit access to the PID by unauthenticated devices and/or users of the mobile device 110 and/or the medical equipment 170 that lack authorization to access PID. In some cases, encryption may enable access to de-identified physiologic data.

The medical equipment 170 may package data in one or more predetermined message configurations or formats for transmission to the mobile device 110. In some implementations, real-time or near real-time data (e.g., case information derived from patient interface devices 180) can be transmitted as streaming data in a JavaScript Object Notation (JSON) format sent over a WebSocket. Historical and bulk data transfers, in some examples, can be transmitted as Representational State Transfer (REST) data in JSON-formatted messages. Both types of message communications (streaming data and REST data) can occur over a transport layer security (TLS) connection, which can use a TCP/IP protocol. The TCP/IP protocol can be provided over Wi-Fi or Bluetooth physical media. When data transfer occurs in real-time or near real-time, case information is simultaneously displayed at the mobile device 110 and the medical equipment 170 or an amount of latency for displaying the case information at the mobile device is less than a predetermined threshold.

The medical supply database 816 may include information about some or all of the medical supplies available in the transport and/or the emergency environments. In an implementation, the medical equipment 170 may populate the medical supply database 816. In an implementation, an agency or emergency crew may populate and/or update the medical supply database 816 via data input to the one or more devices of the computing resources 999 on which this database is provided. The CSG engine 150 may receive a medical supply inventory 815 from the medical supply database 816 via a communicative coupling with the one or more devices of the computing resources 999 in which this database is provided. In some examples, the number of items and/or number of types of items in the device and supply inventories may be between 40-80 items and/or types of items. The medical supply inventory 815 may include available medications and/or may be a running inventory that is updated in real-time as care is provided. The medical device inventory 805 may indicate the availability of devices or services, like blood chemistry analytics, lactate monitoring, capnography, blood glucose tests, and telemetry, which vary amongst various EMS agencies and crews.

The caregiver training database 826 may include information about responder training for an agency or set of agencies. The CSG engine 150 may receive caregiver skill records 825 from the caregiver training database 826 via a communicative coupling with the one or more devices of the computing resources 999 in which this database is provided. In an implementation the CSG engine 150 may obtain the caregiver skill records 825 via entries to the mobile device 110 by the EMS crew. In an implementation, the caregiver training database 826 may be integrated and automatically populated by the dispatch service 130. The caregiver skill level indicates the scope of practice for a particular caregiver. From this information, the CSG engine 150 may determine the skills associated with the EMS crew assigned to the emergency event. In an implementation, the CSG engine 150 may cross-reference the caregiver skill record data 825 with the medical device inventory 805 and the medical supply inventory 815 to determine which medical device interventions are available for the victim based on the ability of the caregivers to utilize the available medical equipment. In general, the scope of practice for various certification levels (e.g., emergency medical responder (EMR), emergency medical technician (EMT), advanced emergency medical technician (AEMT), and paramedic) may vary according to local state or agency protocols. The level of medical skill used to treat victims experiencing life-threatening illnesses or injuries may be broadly grouped as basic life support (BLS) and advanced life support (ALS). EMTs, paramedics, nurses, or sometimes qualified bystanders can provide BLS whereas ALS requires more advanced medical training and is limited to paramedics, physicians, sometimes nurses, or other caregivers that authorized by training to provide ALS. Generally, a BLS provider may not perform invasive procedures and may only administer a limited set of medications. Conversely, an ALS provider may perform invasive procedures and administer a wide array of medications. As other examples, a caregiver with BLS certification may not provide a CPAP intervention whereas a caregiver with ALS certification may provide the CPAP intervention. As other examples, nebulizer treatment, endotracheal suctioning, oral suctioning, intramuscular injection, peripheral venous access require AEMT or paramedic and endotracheal intubation requires paramedic. A particular EMS unit may be a BLS unit or an ALS unit depending on the training level of the personnel and the medical equipment available. An ALS unit generally has at least one paramedic and may be equipped with, for example, advanced airway equipment, a cardiac monitor/defibrillator, IV fluids, a wide array of medications, etc. These items are typically not available with a BLS unit as such a unit is unauthorized to perform procedures requiring this type of equipment.

With regard to defibrillators and other medical equipment, an ALS device will be configured to enable a caregiver to manually control device settings and therapy administrations and to provide a large array of measured and monitored patient physiologic data. This physiologic data generally requires the use of an array of physiological sensors. In contrast, a BLS device will be configured to limit a caregiver's control of the device and provide therapy based on automated determinations from algorithms programmed into the device that a therapy is needed. The caregiver generally cannot override the automated determination in a BLS device. Further, the BLS device provides minimal or no monitored physiologic data beyond what is needed to provide the automated therapy. An ALS defibrillator is configured to allow an ALS provider to monitor a patient's heart rhythm and other vital signs and manually intervene if the provider or the defibrillator algorithms determine that a shock is advised. In contrast, a BLS defibrillator, often an automated external defibrillator (AED) is configured for public access and requires a finding by the device itself that a shock is advised. Thus, the BLS device may not allow a caregiver to manually administer a shock if the device algorithm does not advise shock. Manual administration enables the device to administer a shock in response to a caregiver pressing the shock button even when the heart rhythm analysis algorithm does not advise shock. This is because an ALS provider may determine based on their own advanced medical training and the physiologic data provided by the ALS defibrillator that a shock should be delivered at a particular time. The ALS device allows this feature. Usually, the ALS defibrillator is configured to monitor and display patient vital signs such as heart rate and blood pressure along with pulse oximetry and capnography measurements, twelve lead ECG and may provide pacing and cardioversion along with CPR coaching for compressions and ventilations. In contrast, a BLS defibrillator, such as an AED, is generally not configured to monitor and display patient vital signs such as heart rate and blood pressure along with pulse oximetry and capnography measurements, twelve lead ECG or provide pacing. Finally, the ALS defibrillator generally enables a provider to adjust shock parameters such as the energy whereas the shock parameters in a BLS defibrillator are preconfigured and not adjustable by the caregiver. In some cases, an ALS defibrillator may offer both of a BLS mode and an ALS mode. However, a BLS defibrillator like an AED may not offer the ALS mode.

The identification of a device, a caregiver, and/or an EMS unit as ALS or BLS may provide contextual data that the CSG system 100 may use to determine care and workflow guidance. This distinction provides information on equipment and procedures that are available to the patient. This distinction may also inform a decision about transport because the determination to transport or treat on scene may depend on the equipment and procedures available to the patient.

As one example, if an ALS defibrillator is in use, because the technical capabilities of this device differ from that of a BLS defibrillator, the workflow guidance as to use of the device and/or use of data collected by the device will be different from that of a BLS defibrillator. As one example, the CSG system 100 may not provide guidance for capnography nor include capnography data in a protocol or next best step evaluation if the defibrillator is a BLS defibrillator rather than an ALS defibrillator. Furthermore, the level and type of guidance required by an ALS provider will be more advanced and complex than that for a BLS provider. The next best step and protocol selection algorithms may account for this variation in skill levels. Additionally, where a unit is identified as ALS or BLS, the CSG system 100 may infer or determine that certain equipment and procedures are available or unavailable just based on the level of authorized practice. Similarly, to the equipment and personnel examples, the identification of an EMS unit as ALS or BLS may inform the next best step and protocol evaluations.

Based on the medical device inventory 805, the medical supply inventory 815, and/or the caregiver skill record 825, the CSG engine 150 may identify a need for additional and/or different medical devices and/or medical supplies. For example, the CSG engine 150 may evaluate the emergency event notification information 135 in the transport environment and/or evaluate the notification information 135 along with the environmental data 186 and/or the physiologic data 185 in the emergency environment (e.g., as shown in FIG. 3A) and determine based on, a presenting condition of the victim, a medical status of the victim, a caregiver skill level (e.g., from the caregiver skill record 825), and/or a location of the emergency environment (e.g., from the location and navigation data 835), that additional and/or different medical devices and/or supplies are necessary for adequate care of the victim. In some cases, an obese, pregnant, or pediatric victim may require additional and/or different medical devices and/or supplies. In some cases, the location may indicate particular weather conditions, environmental hazards, and/or other factors relevant to victim care.

The CSG engine 150 may generate a request for the additional and/or different medical supplies and/or medical devices and/or additional caregivers with a different or more advanced scope of practice. The CSG engine 150 may provide this request to the caregiver 103 (e.g., via the CSG UI 155) and/or may communicate directly with dispatch or another source of support outside of the transport and/or emergency environments. In various implementations, another crew or delivery system, including, for example a drone delivery service, may bring the additional supplies and/or devices to the emergency scene 120.

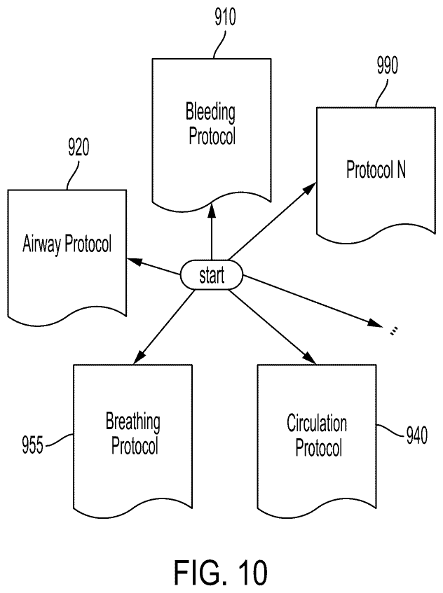

The CSG engine 150 may evaluate protocols (e.g., as discussed further in regard to FIG. 10) in light of available equipment and select one or more next best steps for the caregivers based on the available equipment. The evaluated protocols may include trauma protocols. For example, a protocol may follow one path if ultrasound imaging is available for chest examination and a different path if only a stethoscope is available. As a further example, location may determine the method of patient transport which may in turn determine a next best step for patient care. For instance, if a helicopter would need to land in order to provide CPR and the destination is relatively close, the next best step may be to proceed with patient transport and delay CPR. However, if an ambulance can pull over to the side of a road to administer CPR and the destination is relatively far, the next best step may be to delay transport and administer CPR.

In addition to the available equipment, the CSG engine 150 may consider the caregiver skill level and scope of practice together with the equipment availability. For example, if both of a stethoscope and an ultrasound imaging device are available but the caregiver skill level does not enable them to utilize ultrasound, then, despite availability, the CSG engine 150 may guide the caregiver to the stethoscope examination. Alternatively, the CSG engine 150 may be able to compensate for caregiver skill by guiding the caregiver through a procedure that would be inaccessible to the caregiver in the absence of CSG. This guidance may include telemedicine guidance if a network connection is available.

Referring to FIG. 2D, examples of medical equipment are shown. A quantity of each component in FIG. 2A is an example only and other quantities of each, or any, component could be used. The medical equipment 170 may include medical devices and medical supplies. For example the medical devices may include one or more of a patient monitor 680, a defibrillator 685 (e.g., an automated external defibrillator or a patient monitor/defibrillator), a trauma kit 665, an ultrasound imaging device 690, an automated compression device 695, blood chemistry analytics device(s) 655 (e.g., for lactate and/or glucose monitoring and measurements), a medication delivery system 670, and/or a ventilation system 675.

In an implementation, the trauma kit 665 may include an integrated tablet or other mobile device. In an implementation, the integrated tablet or other mobile device may provide the CSG UI 155 and/or may provide supplementary information or information specific to the contents of the trauma kit. The trauma kit 665 may communicatively couple to the CSG engine 150 to provide supply inventory information and/or supply removal information. In an example, the integrated tablet or mobile device may provide a caregiver UI. This caregiver UI may enable the caregiver to enter physiologic data for the patient. For example, the caregiver UI may be a software application that provides interactive prompts, medical care instructions, and other guidance information. The software application may generate a time-stamped record of caregiver actions based on user entries to the software application. These entries may include physiologic data for the patient. In some cases, the physiologic data may include observed data rather than measured data. For example, observed data may include observed bleeding, burns, laceration, mental state, skin color, sprains, contusions, broken bones, pupil dilation, etc. This observed data may include the existence, body location, and/or extent of the observed condition. Observed data may further include scores such as, for example and not limiting of the disclosure, results of a Rapid Trauma Assessment, an Injury Severity Score (ISS), a triage score, a Glasgow Coma Score (GCS), an Abbreviated Injury Scale (AIS), a Trauma and Injury Severity Score (TRISS), a quick Sequential Organ Failure Assessment (qSOFA), a Lactate enhanced qSOFA (LqSOFA), or other score or assessment used by a caregiver to summarize and characterize a patient's medical state. Measured physiologic data may include body temperature, pulse, blood pressure, etc. Additionally, or alternatively, various items of medical equipment within the trauma kit may be communicatively coupled to the integrated tablet or other mobile device and may transmit or otherwise provide measured physiologic data to the integrated tablet or other mobile device. The integrated tablet or other mobile device may provide received physiologic data (either from caregiver entry or as communicated by the medical equipment) to the CSG engine 150.

In an implementation, the defibrillator 685 may be coupled to a companion mobile device. For example, the defibrillator may be a basic life support (BLS) device or an advanced life support (ALS) device. The companion mobile device may be a tablet that is pre-configured to communicatively couple with the defibrillator. The tablet may provide the device view, working view, trend view, and CSG view windows described herein at least in regard to FIG. 14A-1. In an implementation, the companion mobile device may be a tablet computing device that is pre-configured to communicatively couple with the ALS defibrillator device and to provide a view of a user interface of the ALS defibrillator in real-time at a display screen disposed at the tablet computing device. This view may be the device view described herein. The medical equipment 170 may further include an electronic stethoscope, a SpO2 monitor, an ECG chest patch monitor, a transport monitor, a defibrillator module, an infusion pump, an oxygen supply, CO monitoring equipment, CO2 monitoring equipment, near infrared spectroscopy (NIRS) equipment, etc.

The medical equipment 170 may further include medical supplies 660. One or more of the medical supplies 660 may be included in the trauma kit 665. Medical supplies 660 may include, for example, but not limited to, a cervical collar, a backboard, splints, bandages, blankets, a bag valve mask (BVM), an oxygen supply, airways (e.g., oropharyngeal airway (OPA), nasopharyngeal airway (NPA), laryngopharyngeal airway (LPA)), an intubation tube, an IV administration kit, a spinal motion restriction kit, tourniquets, bandages, occlusive bandages, splints, stretchers, a stair chair, a stokes litter, a traction device, a suction device, nasal cannulas, oxygen, manual stethoscopes, blood pressure cuffs, non-rebreather masks, spinal stabilization board(s), personal protection equipment (e.g., masks gloves, etc.), Heimlich valves, infusion pumps, etc. The supplies 660 may further include plasma, red blood cells, topical hemostatic agents, crystalloid fluids, plasma, intravenous fluids, activated charcoal, and/or medications such as acetaminophen, mannitol, naloxone, aspirin, atropine, diazepam, epinephrine, glucagon, etc.

Referring to FIG. 3A, an example of a device and data configuration for a CSG system 145 in an emergency environment is shown. A quantity of each component in FIG. 3A is an example only and other quantities of each, or any, component could be used. The emergency environment 120 includes the one or more victims 101a, 101b, the one or more caregivers 103a, 103b, the medical equipment 170, and the immediate physical surroundings. The emergency environment further includes the transport vehicle 126, the transport environment data sources 165, and emergency environment data sources 190. The transport vehicle 126 and the transport environment data sources 165 become part of the emergency environment 120 upon arrival of the transport vehicle and the emergency crew at the emergency scene. FIG. 3A provides more granular details of the example of the CSG system 145 in FIG. 2A for deployment in the emergency environment once EMS services are on scene and no longer on route.

As shown in FIG. 3A, once on scene, the first responder(s) or other medical caregiver(s) may triage, treat, and possibly transport the victims to a hospital. Various patient conditions and features of the emergency environment may be critical to providing effective care for the victims. The CSG engine 150 may receive data about these various conditions and features from the emergency environment data sources 190. Collectively, this information may be referred to as emergency environment data 195. In addition, the CSG engine 150 may receive physiologic data 185 from medical devices included in the medical equipment 170. The caregiver 103 may provide medical care to the victim 101 by coupling medical equipment 170 to the victim via patient interface devices 180.

The emergency environment data 195 may include information about the victim, attributes of the emergency scene, and other contextual information that is relevant to treatment of the victim.

Referring to FIG. 3B with further reference to FIG. 3A, examples of environmental data sources are shown. A quantity of each component in FIG. 3B is an example only and other quantities of each, or any, component could be used. The CSG engine 150 may receive the emergency environment data 195 from one or more environmental data sources 190. The environmental data source(s) 190 provide contextual data for the victim 101 and the emergency scene 120.

In an implementation, one or more of the touchscreen 106, the keyboard 582, the mouse 583, the clock 580, the camera 581, the scanner 586, the speaker 585, microphone 584, the heads up display 16, the location device 596, and the context utilities 599 may be disposed at the mobile device(s) 110. The mobile device(s) 110 may also be an environmental data source 190 and provide environmental data 195. The context utilities 599 may include, but are not limited to, a calendar application, a contacts application, a weather application, a mapping application, etc. The speaker 585 and the microphone 584 may be combined into a single device such as the earpiece 595.

The CSG UI 155 is shown in FIG. 3A on the mobile device 110 and shown in FIG. 3B as part of the environmental data sources 190 that provide emergency environment data 195 to the CSG engine 150. The CSG UI 155 may both collect emergency environment data and provide that data to the CSG engine 150 and may receive output from the CSG engine 150, such as caregiver instructions to display at the CSG UI 155. The CSG UI 155 is graphically represented in FIG. 3A as separated from the emergency environment data sources 190. However, the CSG UI 155 may be at least one of the environmental data sources providing information to the CSG engine 150. As discussed below, one or more of the environmental data sources 190 may be disposed at the mobile device 110 along with the CSG engine 150.

The environmental data source(s) 190 may monitor and/or collect environmental data 195 about the emergency environment 120 and the victim 101 during medical care. The environmental data 195 may include contextual and/or observational data from the emergency environment 120 and the caregiver 103 that is available on-site at the point of care for the victim 101. Such data may include, for example, image, sound, location, and time information.

The environmental data source(s) 190 may include one or more devices that enable the CSG engine 150 to receive information from the caregiver 103 and/or from the emergency environment. For example, the microphone 584 and/or the touchscreen 106 may enable the caregiver 103 to provide caregiver observations to the CSG engine 150. Caregiver observations capture conditions, parameters, etc. of the victim 101 and/or the emergency environment 120 that may not be measurable by or may not be practical to measure with medical equipment or another electronic and/or mechanical device. The caregiver 103 may verbalize these observations and the CSG engine 150 may receive these verbalized observations as audio input via the microphone 584. Alternatively, or additionally, the caregiver may input observations via a user input device such as the touchscreen 106, a keyboard 582, or a mouse 583. In an implementation, the touchscreen 106 may provide the CSG UI 155. The CSG UI 155 may provide various menus, prompts, etc. to facilitate data entry.

As examples of caregiver observations, a triage evaluation may include observation of patient responsiveness, respiratory distress, mental confusion, visible wounds, bleeding, skin color, pupil dilation, broken bones, bruises, rigidity, guarding, pallor, etc. Other on scene observations may include hazards to the EMS crew such as downed power lines, hazardous chemicals, fire, spilled fuel, crime scene, possibility of violence, etc. Observations may further include equipment available on scene, presence of firefighters, police, and/or EMS responders, injury context observations such as airbag deployment, seat belt use, rollover, loose objects in automobile that may have caused injury, windshield damage caused by patient impact, and/or type of collision (e.g., head on, rear end, rollover, rotational impact) etc. These injury context observations may indicate particular likely physical injuries. Patient observations may include examination findings such as, for example, patient respiration characteristics like uneven lung sounds, chest wall motion, etc. As other examples, the caregiver 103 may observe that the victim 101 was the driver of a car involved in a collision. The caregiver 103 may further observe that the victim 101 appears confused and anxious, has a blue skin tone, and complains of chest pain. Additionally, the caregiver 103 may observe lacerations and bruising on particular parts of the victim's body. As another example, the caregiver may observe that the victim is located at the side of a highway at night in a snowstorm, has been thrown from a car, is confused, anxious, and blue in appearance complaining of chest pain with lacerations on the forehead and left arm and bruising on the right leg. Additionally, the caregiver may receive injury and other victim information verbally in a conversation with a conscious victim, a bystander, another caregiver, a family member etc.

Referring to FIGS. 3C-1, 3C-2, and 3D, examples of non-verbal data entry to the CSG engine are shown. For example, as shown in FIGS. 3C-1 and 3C-2, the caregiver 103 may enter data through a touchscreen display of the CSG UI 155. FIGS. 14A-1 through 14D-8 provide further examples of the CSG UI 155. As another example, the caregiver 103 use the heads-up display device 725 to access a virtual user interface 16 and provide entry via a gesture 18. In an implementation, as shown in FIG. 3C-2, user selections define exclusion and/or inclusion criteria 301 for protocol selection and workflow guidance generation by the CSG engine 150.

Referring to FIG. 3E, the CSG engine 150 may receive verbalized observations and conversations as audio input. For example, the caregiver 103 may provide the observational information 115 to the CSG engine 150 via a combined speaker/microphone device in the form of an earpiece 595. Based at least in part on this observational information, the CSG engine 150 may provide audible caregiver guidance 52 back to the caregiver via the earpiece 595.

Referring again to FIG. 3B, the environmental data source(s) 190 may include a camera 581 and/or scanner 586, a clock 580, and/or a location device 596. In an implementation, the camera 581 may function as a scanner and the camera and scanner may be a singular device. The location device 596 may be a global positioning system (GPS) device, a cellular network positioning device, or a combination thereof. In an implementation, the environmental data 186 may also include images, associated with the emergency environment 120 as captured by a camera 581 and/or scanner 586. For example, as shown in FIG. 3F, the mobile device 110 may capture an image 12, for example of a laceration, and/or a bar code or quick response (QR) code (e.g., as represented by the QR code 14) via a camera, scanner, or combination device. As another example, the environmental data 186 include time and location information, e.g., from a clock 580 and the location device 596. Such data may indicate that the emergency scene is fifteen minutes from the closest hospital in current traffic conditions. In an implementation, the CSG engine 150 may adjust guidance based on one or more of time and location data, determined travel time to a hospital and determined travel time to a hospital in determined current traffic conditions. For example, the CSG engine 150 may recommend more on-scene and/or in-ambulance procedures for a relatively long travel time and may recommend stabilization without further procedures for a relatively short travel time. This adjustment may be dynamically determined via the input from the environmental data sources 190.

Referring to FIGS. 3G-1-3G-3, examples of various configurations of the environmental data sources 190 are shown. A quantity of each component in FIGS. 3G-1-3G3 is an example only and other quantities of each, or any, component could be used. In FIG. 3G-1, all of the environmental data sources 190 are disposed at the same mobile computing device that provides the CSG UI 155. FIG. 3G-2, one or more of the environmental data sources 190 are disposed at the same mobile computing device that provides the CSG UI 155 and one or more of the sources 190 are disposed on one or more other devices within the computing resources 999. In FIG. 3G-3, all of the environmental data sources 190 are disposed at devices external to the mobile computing device 110 that provides the CSG UI 155. In all of these cases, the CSG engine 150 may be disposed at one of more of the computing resources 999.

Referring again to FIG. 3A, one or more items of medical equipment 170, for example one or more medical devices (e.g., the defibrillator 685, the patient monitor 680, the trauma kit 665, the ventilation system 675, the automated compression device 695, the ultrasound device 690, the medication delivery device 670, or the blood chemistry analytics device(s) 655) may include a communications interface 133 that enables the associated medical device to communicatively couple to the mobile device 110 via the communications link 199 and the communications interface 132 of the mobile device 110. The medical equipment 170 may be configured to gather physiologic data from the patient via the patient interface devices 180, analyze this data, and, in some instances, may delivery therapy to the patient (e.g., electrotherapy or respiratory therapy) based on the gathered and analyzed data. In this manner, the CSG engine 150 may receive information, including physiologic data 185, from the medical equipment 170. During deployment of the medical equipment 170, the medical devices may store and/or transmit (e.g., to the mobile device 110, the edge server 129, and/or computing devices in the remote environment 320) one or more medical device case files 188 that include records of data gathered during a patient case and of treatments and/or interventions by the medical device(s) along with records of device settings and operations during the patient case. The CSG engine 150 may also instruct and/or control medical devices to implement clinical interventions and/or provide patient record data and event markers to the medical devices. Additionally, the CSG engine 150 may also request specific patient data from the medical devices. The CSG engine 150 is thus able to analyze the environmental data, the physiologic data, the emergency event notification information, and the transport environment data to provide guidance and instruction.

Referring to FIG. 4, an example of a CSG engine implemented on a computing device is shown. A quantity of each component in FIG. 4 is an example only and other quantities of each, or any, component could be used.

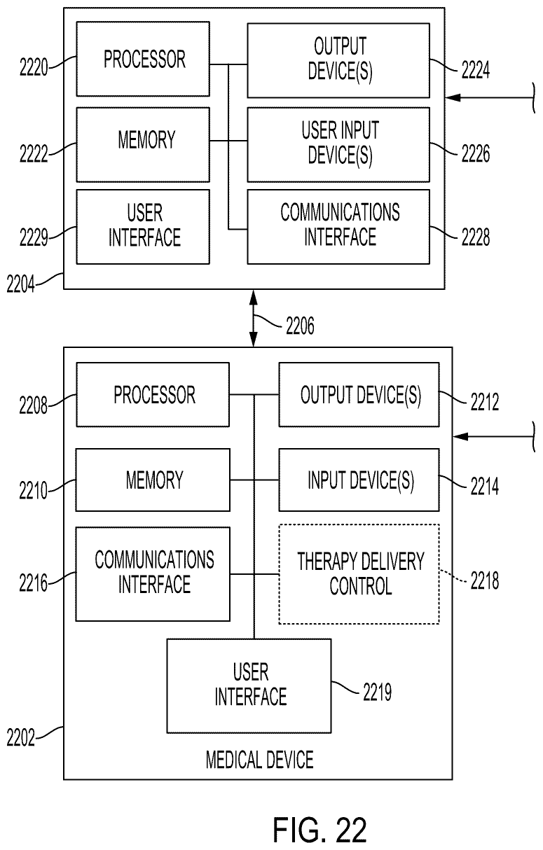

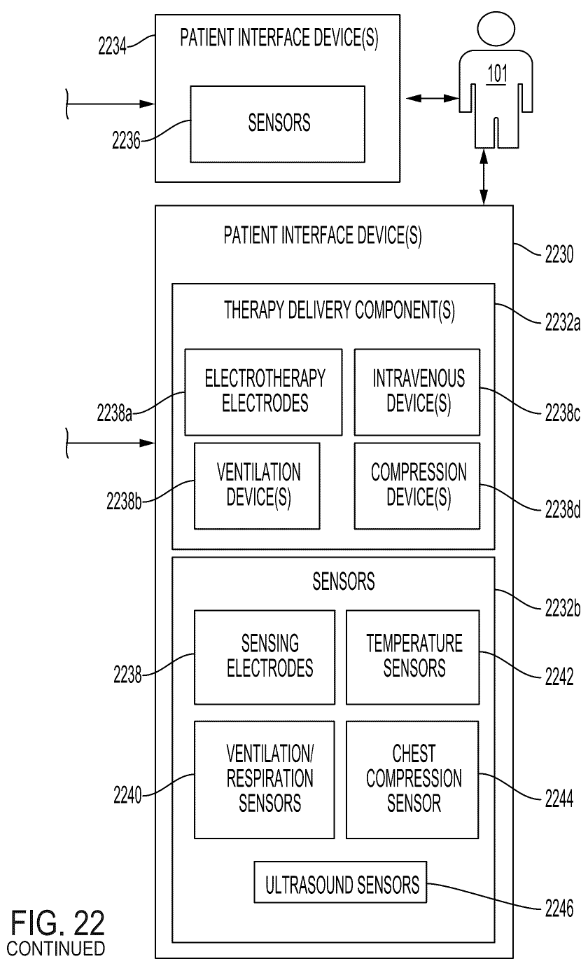

A processor 108 and a memory 109 (i.e., a non-transitory processor readable storage medium) of a mobile device 110 may provide the hardware logic and/or the software logic of the CSG engine 150. The memory 109 may include a CSG algorithm 151 that provides at least a portion of the software logic. In an implementation, the CSG algorithm 151 may be a trauma CSG algorithm tailored specifically to trauma care. The mobile device 110 may further include a display screen 106 that may be a touchscreen. Although shown as a component of the mobile device 110, in various implementations, the CSG engine 150 may be disposed at a medical device (e.g., one or more of the medical equipment 170) or at a remote computing device (e.g., the remote device 310 and/or a cloud server 375 as shown in FIG. 5). In an implementation, the CSG engine 150 may be a distributed resource across multiple physical devices that work together programmatically. The CSG engine 150 may control a CSG user interface (UI) 155 at one or more display screens, audio devices, and/or haptic devices. For example, the CSG UI 155 may provide and/or receive information via the mobile device display screen 106, the speaker 597, the microphone 584, and the camera 581, other environmental data sources 190, mobile devices 110, a medical device user interface (e.g., the user interface 2219 in FIG. 22), and combinations thereof.

In addition to the CSG engine 150 and algorithm 151 as implemented and stored by the processor 108 and the memory 109, the mobile device 110 may provide the CSG UI 155 and may include the communications interface 132. The mobile device 110 may further provide a patient charting application 131. In an implementation, the CSG engine 150 may provide the CSG UI 155 at one or more of the mobile devices 110 and/or at one or more items of the medical equipment 170. The CSG UI 155 may receive information from the caregiver (e.g., measured patient data and caregiver observations) and provide this information to the CSG engine 150. Further, the CSG engine 150 may provide prompts and/or instructions for the caregiver at the CSG UI 155 in order to implement clinical interventions and guide patient care.

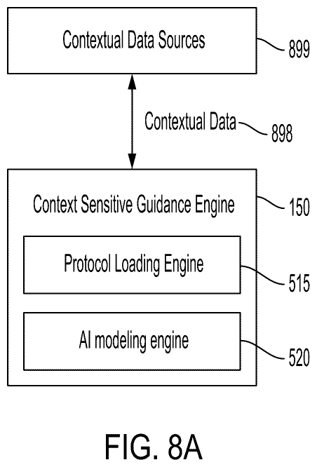

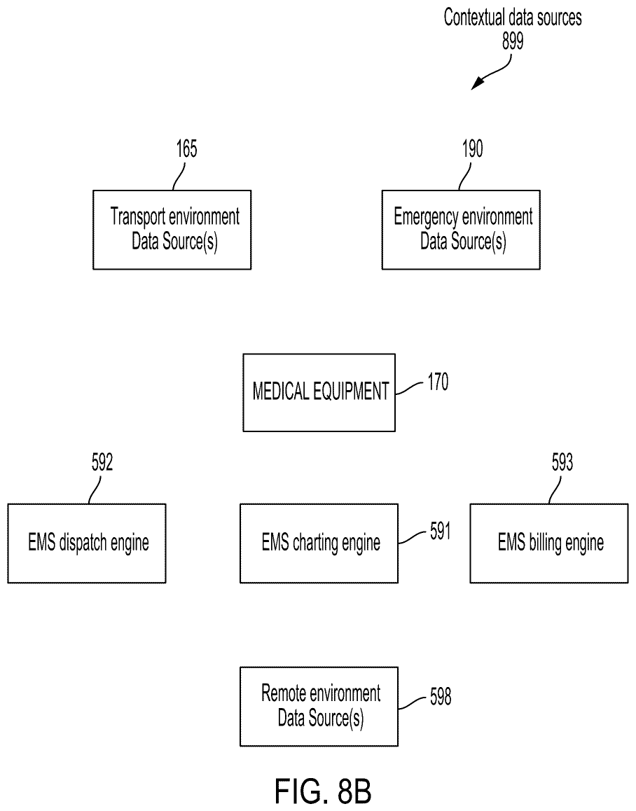

The patient charting application 131 may generate an electronic patient care record (ePCR) as a contemporaneous record of caregiver observations, treatments and interventions provided, physiologic data for the patient, patient transport, transport destination, times and durations of patient care activities, emergency crew identification, patient demographic information, patient health insurance information, etc. The information entered into the ePCR may be entries from the caregiver captured by the mobile device (e.g., touchscreen or keyboard entries, voice entries), entries via one or more input devices (e.g., scanner, microphone, camera, etc.), and/or automated entries from communicatively coupled devices or databases. The application 131 may operate in parallel or in cooperation with the CSG engine 150. In an implementation, the CSG engine 150 may implement the patient charting application 131 and the CSG algorithm 151 through a combined user interface. In various implementations, the CSG engine 150 may provide output to the patient charting application 131. The output from the CSG engine 150 may include information for recordation in the ePCR (e.g., event markers, physiologic data, data evaluations, intervention information, etc.). In an implementation, the CSG engine 150 may reside programmatically within the patient charting application 131. In an implementation, and as discussed in further detail below with regard to FIG. 8A, the CSG engine 150 may include an AI modeling engine 520. The AI modeling engine 520 may receive speech from the caregiver and convert that speech to text and provide the text to the electronic patient care record (ePCR), the medical device case file 188, and/or the CSG engine 150.

Referring to FIG. 5, an example of a CSG system configuration that includes a remote computing environment is shown. A quantity of each component in FIG. 5 is an example only and other quantities of each, or any, component could be used.

The mobile device 110 may communicate with a remote computing environment 320 (e.g., a dispatch center, an emergency room computing device, a telemedicine computing device, a remote server and/or database, etc.) via a communicative coupling 399 with a remote communications network 380 (e.g., a computer network, such as the Internet, and/or a cellular network). The remote computing environment 320 may include one or more medical records databases 390 and/or the cloud server 375. The network 380 enables communications between computing devices in the emergency environment 120 and one or more of the cloud server 375, the medical records database(s) 390, and the telemedicine support 385. In various implementations, some or all of the devices within the emergency environment 120 may be configured to communicate with computing and/or communication devices in the remote computing environment 320 to enable, for example, telemedicine, health history, and data storage services. A long-range wireless network 380 may enable the communicative coupling between one or more devices within the emergency environment 120 and the one or more devices in the remote computing environment 320. In an implementation, the emergency environment 120 may include the edge server 129 as described above in regard to FIG. 2B.

In an implementation, a health information exchange may couple endpoints in the remote environment with one another and/or with computing devices and software applications in the emergency environment 120. The database 390 may include medical records such as an electronic patient care record (ePCR) (i.e., a patient chart, generated during an encounter through a patient charting application 131), an archived patient chart, a record from a health information exchange (HIE) or other regional data base, and/or a hospital electronic health record (EHR).

In an implementation, the CSG engine 150 may incorporate telemedicine provider information in the presence of network connectivity. The remote computing environment 320 may include one or more computing devices 310 to enable telemedicine support 385 from a physician 389. The computing device 310 may be a mobile device, a workstation, or a combination thereof. The computing device 310 may communicate with computing devices in the emergency environment via a communicative connection 398 with the cloud server 375 and/or via a communicative connection 397 independent of the cloud server 375. The mobile device 110 may communicatively couple to a computing device 310 associated with a telemedicine provider 389, such as a nurse, physician, or medical director. In an implementation, the communicative coupling between the CSG engine 150 and the telemedicine support 385 may occur via the cloud server 375 (e.g., a combination of communicative couplings 398 and 399). Additionally, or alternatively, the telemedicine support 385 and the CSG engine may communicatively couple via the network 380 without the involvement of the cloud server 375 (e.g., a combination of communicative couplings 397 and 399). The CSG algorithm 151 may consume this information and/or the CSG UI 155 may provide this information to the caregiver 103. If the connectivity lapses, the CSG engine 150 may provide support to the caregiver 103 consistent with the interrupted guidance from the telemedicine provider until the connectivity is reestablished. For example, if the telemedicine provider is instructing the caregiver 103 on a particular procedure, the CSG engine 150 may continue these instructions based on stored information or machine learning models. The network service engine 330 enables provision of the telemedicine support when the connection is available or reestablished.

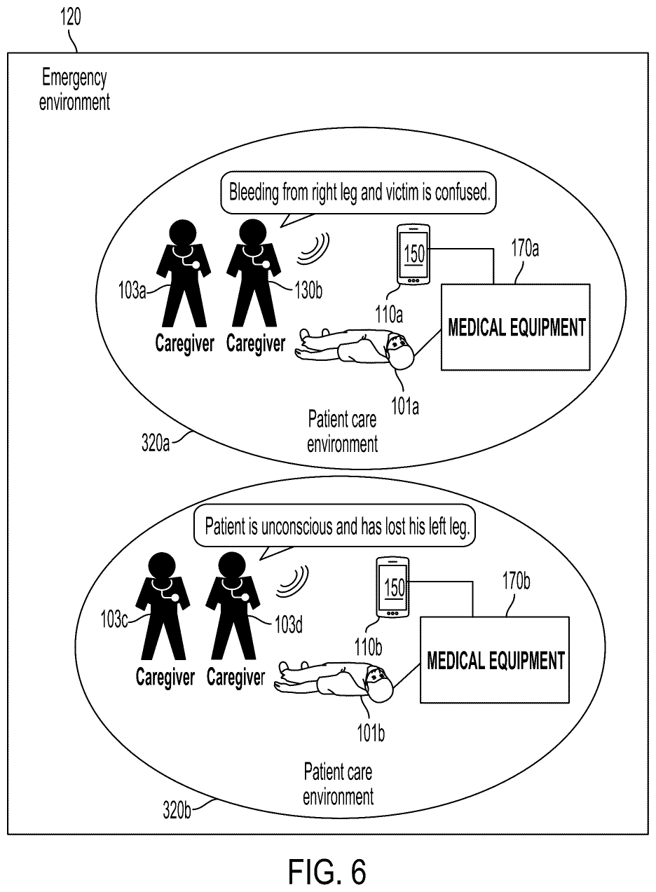

Referring to FIG. 6, an example of patient care environments within an emergency environment is shown. A quantity of each component in FIG. 6 is an example only and other quantities of each, or any, component could be used. The patient care environment is a patient-centric environment in which medical devices, computing devices, and caregivers are focused on the short-term care of a single patient. This environment may be situated, for example, at the scene of a collision or health emergency, on a battlefield or other military setting, in an ambulance, in an emergency room or hospital, etc. The short-term care, for example, acute critical trauma care as described herein, entails identifying one or more clinical presentations that require interventions to stabilize the victim and ensure that the victim stays alive long enough to be moved or transported to a longer-term care environment, such as a hospital.

The constellation of devices connected to and/or receiving information for a single patient, or victim, 101a and/or 101b form a patient care environment, e.g., the environment 320a for the victim 101a and the environment 320b for the victim 101b. Within each patient care environment, all of the medical equipment associated with the single victim forms a medical device suite for a particular victim at the point of care, e.g., the medical equipment 170a for the victim 101a and the medical equipment 170b for the victim 101b. The medical device suite at the point of care may be configured to provide acute care clinical interventions for trauma. In an implementation, the mobile computing devices 110a and 110b are each configured to communicatively couple with the medical equipment 170a or 170b in a respective medical device suite such that the mobile computing devices and the respective medical device suite are associated with a same victim. Thus, all of the physiologic parameters and device information provided at each mobile device 110 is associated with that same single patient. As such, data view windows at the mobile device provide data for one patient and the CSG engine 150 provides dedicated guidance for the care of only one patient.

Within each patient care environment 320a and 320b, one or more rescuers or caregivers (e.g., 103B, 103C, 103c, and 103d) attend to a victim (e.g., 101a and 101b). The one or more rescuers may be, for instance, a civilian responder with limited or no training in lifesaving techniques, a first responder with basic skills, such as an emergency medical technician (EMT), police officer, or firefighter, a paramedic or EMS medical director with advanced skills, or a medical professional, such as a physician or nurse. The rescuer may be acting alone or may be acting with assistance from one or more other rescuers. Additionally, each rescuer may attend to more than one victim within an emergency environment. For example, as illustrated in FIG. 2A, the emergency environment 120 may include at least two victims 101a and 101b. In some situations, the caregivers 103B and 103C may attend to both victims and caregivers 103c and 103d may not be on scene.

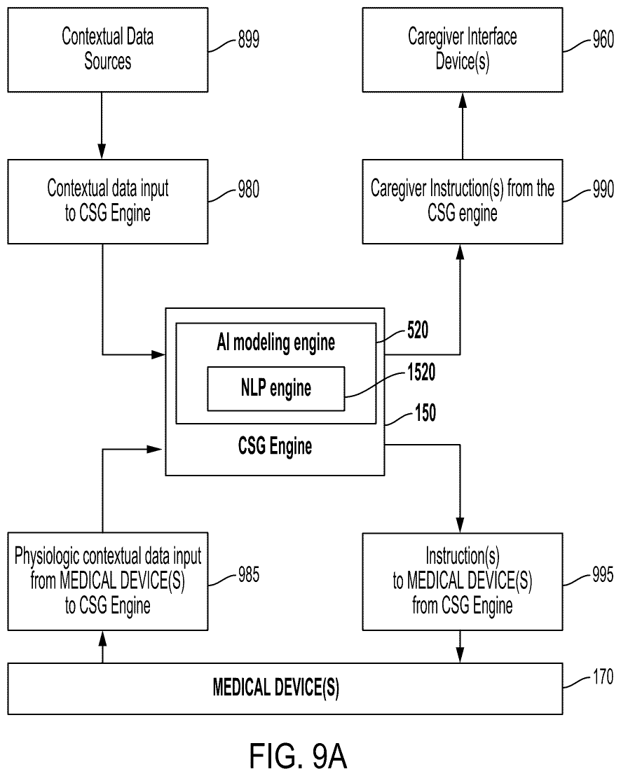

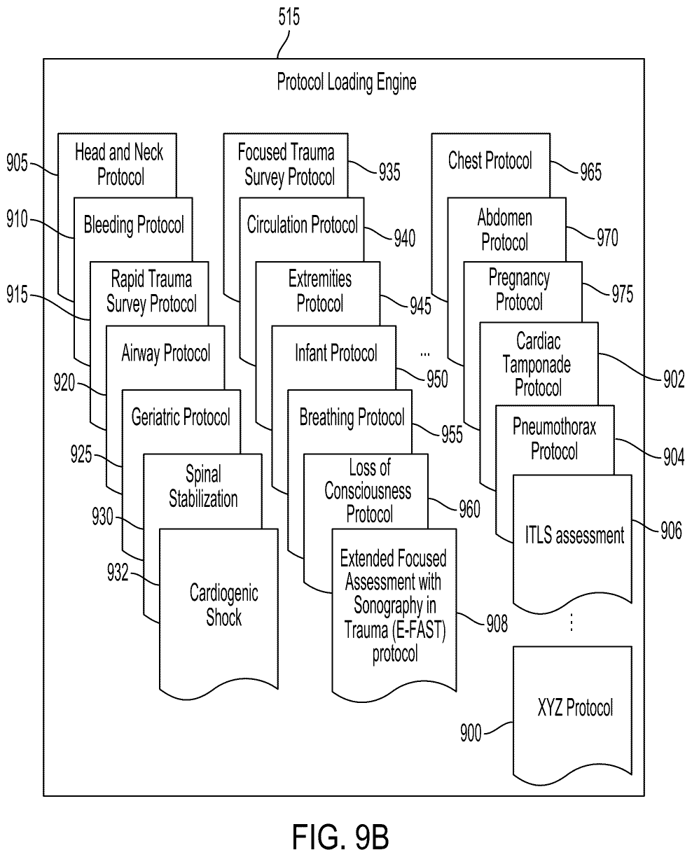

The CSG engine 150 may evaluate multiple protocols in parallel based on the physiologic and environmental data. The protocols are discussed in further detail below in regard to FIGS. 9B, 10, and 11. Based on the evaluation of the protocols, the CSG engine 150 may select at least one action item, which may be an intervention or a treatment. Further, the CSG engine 150 may generate at least one instruction for the caregiver 103 and/or the medical equipment 170. The CSG engine 150 may provide the caregiver instruction to the caregiver (e.g., via CSG UI 155) and/or provide the medical device instruction to at least one medical device 170 in the medical equipment 170.

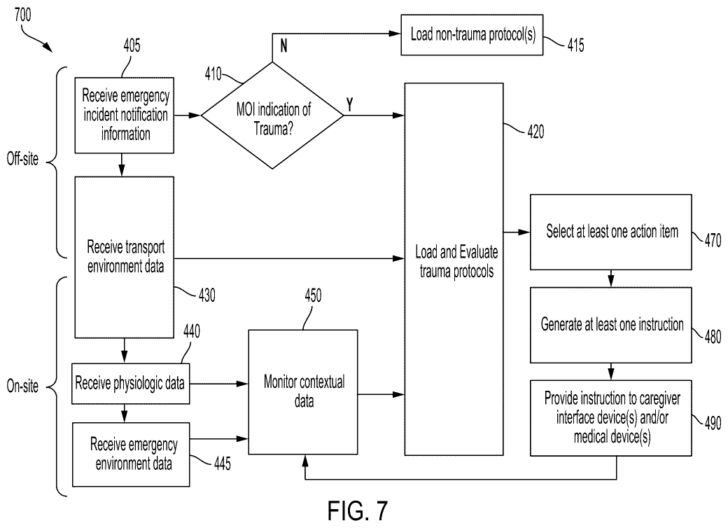

Referring to FIG. 7, an example of a method executed by a CSG system is shown. The method 700 is, however, an example only and not limiting. The method 700 can be altered, e.g., by having stages added, removed, rearranged, combined, and/or performed concurrently.

As discussed above in regard to FIG. 2A, off-site and prior to arrival at the emergency scene 120, the CSG engine 150 may receive 405 the emergency incident notification information 135 from the emergency dispatch service 130. Additionally, off-site and on-route to the emergency scene 120, the CSG engine 150 may receive 430 the transport environment data 160. In some instances, the emergency incident notification information 135 may be sufficient for the CSG engine 150 to determine 410 if a trauma injury to the victim is possible. In an implementation, where a trauma injury is possible, the CSG engine 150 may generate at least one preliminary instruction for the caregivers and/or a medical device prior to arrival of the caregivers at the emergency scene based on the emergency incident notification information 135. The emergency incident notification information 135 and the transport environment data 160 are portions of contextual data available to the CSG engine 150.

Often, the emergency incident notification information 135 includes a mechanism of injury (MOI). In an implementation, the electronic patient care record (ePCR) generated by the first responders may also be a source of the MOI. The CSG engine 150 may associate the MOI with a high probability of trauma. For example, an MOI of a motor vehicle collision, a fall, an explosion, or a structural collapse would all carry with them a high probability of traumatic injury. Conversely, an MOI of cardiac arrest or asthma induced respiratory distress may carry a low probability of traumatic injury. The probability may be low but non-zero because, for example, it is possible that a cardiac arrest victim may sustain a traumatic head injury due to a collapse during the cardiac arrest.

If there is no indication of possible trauma, the CSG engine 150 may load 415 non-trauma protocols based on the MOI. For example, the non-trauma protocols may include a cardiac arrest protocol or a respiratory distress protocol. If there is no indication of trauma, the CSG engine 150 may exclude trauma protocols. If there is an indication of traumatic injury, then the CSG engine 150 may invoke the CSG engine 150. The CSG engine may load and evaluate 420 trauma protocols. If there is an indication of a combination of traumatic and non-traumatic patient conditions, then the CSG engine 150 will invoke multiple subsystems to load a combination of trauma protocols and non-trauma protocols. Additionally, if contextual data subsequent to the initial MOI evaluation indicates a traumatic and/or a non-traumatic injury, the CSG engine 150 may invoke the appropriate subsystem dynamically as the victim care progresses. For example, a victim of blunt trauma may subsequently experience respiratory distress.

In an implementation, the protocol loading engine 515, as discussed below in regard to FIG. 9B, may include (e.g., select a protocol to load) or exclude (e.g., not select a protocol to load) protocols based on the exclusion and inclusion criteria 301. In some examples, and referring to FIG. 7, the CSG engine 150 may load non-trauma protocol(s) 415 and/or the load trauma protocols 420 based on unselected or otherwise unindicated patient symptoms, presentations, or conditions. For example, the CSG UI 155 may provide a list of candidate patient conditions (e.g., as shown for example in FIG. 3C-2). The caregiver may select one or more of the candidate patient conditions. The candidate patient conditions that the caregiver does not select remain as unselected candidate patient conditions. Based on the unselected conditions or mechanisms of injury may form the exclusion and inclusion criteria 301. For instance, examples are provided at least in FIGS. 3C-1, 3C-2, and 3D of caregiver selections or indications of various patient conditions. In the example of FIG. 3C-1, “trauma” is selected but “cardiac,” “respiratory distress,” “gynecology,” and “mass casualty” are unselected and may be excluded. In the example of FIG. 3C-2, “wheezing” and “patient airway are selected and “MOI Trauma,” “Anaphylaxis/Urticaria,” “Pulmonary Edema/Crackles,” “Unequal Airway,” “Cardiac,” and “Near Death” are unselected and may be excluded. In the example of FIG. 3D, “fall” is selected, but “drowning” and “assault” are unselected and may be excluded.

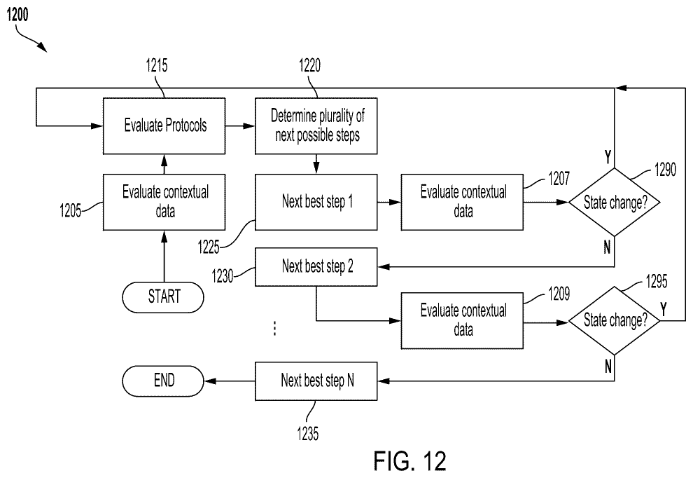

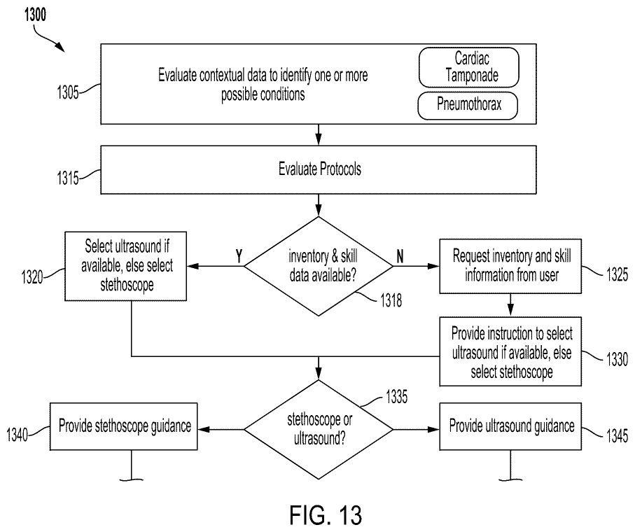

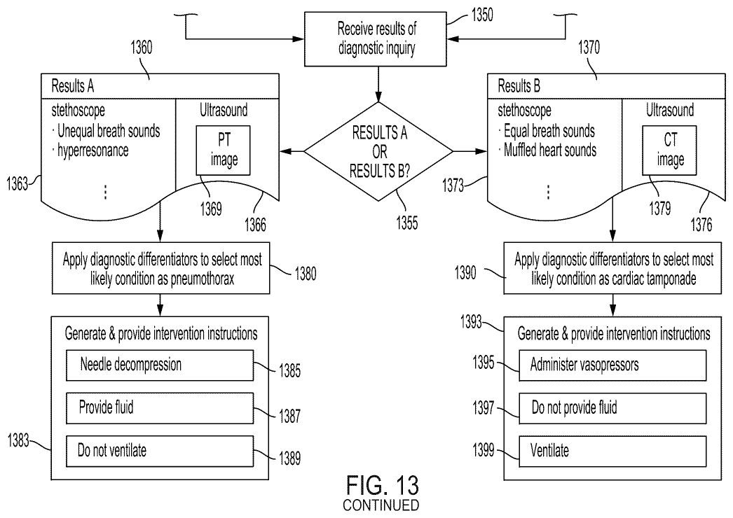

In an implementation, the criteria 301 may be part of the contextual data evaluated in determining a next best step of treatment for a patient. For example, at the stages 1205, 1207 and 1209 as shown for example in FIG. 12, the exclusion and inclusion criteria 301 may be included in the evaluated contextual data. Similarly, at these criteria may be included in the evaluation discussed at the stage 1305 as shown for example in FIG. 13.

Based on the exclusion and inclusion criteria 301, the CSG engine 150 may remove, or exclude, a protocol or workflow guidance step or include a protocol or workflow guidance step. For example, if a caregiver selects a female gender for a patient and selects a patient age between 13-55 (e.g., as illustrated in FIG. 3C-2), these selections indicate that the patient could potentially be pregnant. Therefore, inclusion of these criteria may cause the CSG engine 150 to include the pregnancy trauma assessment protocol. Conversely, exclusion of these criteria (e.g., male gender or female gender with age below 13 or above 55) may cause the CSG engine 150 to exclude the pregnancy trauma assessment protocol. Other examples of selections that may function to exclude or include a particular protocol are selections of a geriatric patient, an obese patient, or a pediatric patient. These categories of patients may require specialized workflows for proper treatment of the patient. As another example, indication of a mass casualty event may trigger inclusion of different protocols than a single patient event. As a further example, the protocol selection engine 515 may exclude a spinal motion restriction protocol unless the caregiver selects one or more of the following: head wound, neck wound, neck pain or tenderness, or lack of motion in extremities. As yet another example, the protocol selection engine 515 may load one or more protocols based on initial patient presentations but then add or change those protocols based on the exclusion/inclusion criteria. For instance, a patient may initially present with chronic obstructive pulmonary disease (COPD) and trouble breathing. Based on these presentations, the CSG engine 150 may include a bronchospasm workflow or protocol. However, if the caregiver selects or otherwise indicates that the patient is near death (e.g., selects “near death” in the example in FIG. 3C-2) and/or lacks a patent airway (e.g., does not select “patent airway” in the example of FIG. 3C-2), then the CSG engine 150 would include and prioritize an intubation workflow or protocol. The intubation is the next best step and may be necessary to prevent death. The priority in such an example would be to prevent death and then subsequently treat for bronchospasms. As another example, if the caregiver selects or otherwise indicates “unequal air entry,” then the CSG engine 150 may include a tension pneumothorax or cardiac tamponade protocol or exclude these protocols in the absence of a selection of unequal air entry.

In an implementation, the CSG engine 150 may exclude certain protocols based on the terms or phrases missing from voice data provided, for example, as shown in FIG. 3E and as processed by the NLP engine 1520 as shown in FIG. 9A. Although the exclusion and inclusion criteria 301 are shown in the examples of FIGS. 3C-1 and 3C-2 as items on a selectable menu list, this is an example only and limiting of the disclosure. The CSG engine 150 may exclude or include protocols based on any contextual information received as described in various implementations herein.

Based on models trained on past history data for traumatic injuries, the CSG engine 150 may determine a probability of a trauma injury. For example, a high-speed collision, a severed limb, or multiple stab wounds would increase the likelihood of a traumatic injury. On the other hand, if the MOI is cardiac arrest, then without other indications of traumatic injury, the CSG system would load 415 a non-trauma protocol(s), in this case, a cardiac arrest protocol. In an implementation, CSG engine 150 may determine a likelihood of trauma based on a location indicated by the emergency incident notification information 135. For example, an emergency environment 120 located along a highway is more likely associated with traumatic injury than a private home or a senior center. Based on a determination that traumatic injury is likely, the CSG engine 150 would load and evaluate trauma protocols based on the off-site information. As another example, a traumatic injury that is likely to cause hemorrhage coupled to cardiac arrest may require an adjustment to a CPR protocol based on a trauma protocol because chest compressions in the presence of hemorrhage may exacerbate the bleeding. Therefore, the CSG system may work with multiple protocols in parallel to manage a multi-system patient condition.

Once on-site at the emergency scene 120, the first responders may discern the MOI and/or additional emergency event information through their own personal or collective observation of the victim and of the emergency scene. The first responders may provide this contextual information to the CSG engine 150 either verbally (e.g., through the AI modeling engine 520) or via touchscreen entry to the CSG UI 155. Additionally, once on-site, the CSG engine 150 may receive 440 physiologic data from the medical equipment 170 and may receive 445 emergency environment data from the environmental data sources 190 (e.g., as shown for example in FIG. 3B). The verbal caregiver data, the data entered at the CSG UI 155, the physiologic data, the emergency environment data are further portions of the contextual data available to the CSG engine 150.

For example, the medical equipment 170 may include a trauma kit (e.g., the trauma kit 665 shown in FIG. 2D) communicatively coupled to the CSG engine 150. The trauma kit may provide data indicative of trauma. For example, the trauma kit may store and transmit data regarding supplies that have been removed from the kit to treat the victim. Thus, medical supply inventory information provided to the CSG engine 150 may be indicative of supplies used for trauma treatment that have been removed from the trauma kit in the emergency environment. In one or more examples, the CSG engine 150 may provide one or more caregiver instructions and/or one or more medical device instructions based on what has been removed from the trauma kit 665 and/or based on what remains in the trauma kit 665. For example, if a caregiver removes a tourniquet from the trauma kit 665, the CSG engine may provide instructions for tourniquet use and/or alerts associated with tourniquet use. For instance, chest compressions may be dangerous if there is external bleeding as the compressions prior to tourniquet application may exacerbate bleeding. As another example, if the caregiver removes allergy medications from the trauma kit 665, the CSG engine 150 may provide a medical device instruction to monitor and/or adjust respiratory therapy based on allergy induced respiratory distress. As an additional example, the trauma kit 665 may detect a removal of a cervical collar and provide this removal information to the CSG engine 150. In response, the CSG engine 150 may provide instructions for cervical collar use and/or alerts associated with cervical spine stabilization. The use of the cervical collar and spine support device may arise, for example, in the case of an occupant of an automobile involved in a collision. The instructions provided by the CSG engine 150 may include instructions for manual stabilization of the C-spine, application of the cervical collar, and use of a spine support during extraction of the occupant from the vehicle and from the trauma scene. Additionally, the CSG engine 150 may provide instructions for conducting a rapid trauma assessment. As a further example, the CSG engine 150 may modify a workflow guidance based on a lack of available equipment in the trauma kit 665. As an example, a workflow guidance for a broken bone may specify a splint. However, if a splint is not available in the trauma kit, the CSG engine 150 may provide workflow guidance for using an elastic bandage in a manner that will properly stabilize the broken bone in the absence of a splint. As another example, a workflow guidance may recommend intubation with an endotracheal (ET) tube. The CSG engine 150 may receive inventory information from the trauma kit 665 indicating that the ET tube is not available and that a bag valve mask is available in the trauma kit. Based on this inventory information, the CSG engine 150 may provide an alert and/or an alternate workflow guidance for a bag valve mask ventilation. As yet further examples of actions by the CSG engine 150 based on inventory in a trauma kit 665, an inventory indication of removal of a medication from the kit by a caregiver may cause the CSG engine 150 to provide at the CSG UI contraindication information, drug interaction information, dosage information, and/or dosage time stamp markers for recordation of medication administration; an inventory indication of removal of a needle thoracotomy kit may cause the CSG engine 150 to provide at the CSG UI prompts for point of care ultrasound (POCUS) in order to confirm or disconfirm a tension pneumothorax; an inventory indication of removal of a lactate may cause the CSG engine 150 to provide at the CSG UI prompts for component data of an quick lactate enhanced Sequential Organ Failure Assessment (LqSOFA) score. As another example, the number of a particular item removed from the kit may cause the CSG engine 150 to provide prompts and assistance as multiple removals may indicate that the caregiver is having difficulty using a particular item. For example, where the probability of a patient requiring two or three tourniquets is low, the removal of two or three tourniquets from the trauma kit may indicate that the caregiver is struggling with proper tourniquet procedures. Thus, after a removal of a predetermined number of tourniquets, the CSG engine 150 may prompt the caregiver to request guidance or may provide the guidance on tightening and/or checking proper placement of a tourniquet.

As another example, the caregiver 103 may couple the victim to a patient monitor and/or a defibrillator. The physiologic data may include vital signs such as SpO2, EtCO2, blood pressure, heart rate, respiratory rate, and ECG and this data may indicate trauma. Early indicators of trauma may include a general impression of a patient based on pupil reactivity, signs of bleeding or blunt trauma (e.g., a bruise or fracture), patient alertness, and general observations of airway, breathing, and circulation. The CSG engine 150 receive the contextual data in real-time as it is generated and/or provided by the medical equipment 170, transport environment data sources 165, and the emergency environment data sources 190. The CSG engine 150 may monitor 450 this incoming data for indicators of trauma. For example, the CSG engine 150 may repeatedly analyze this data to see if any of the physiologic data, the transport environment data, and/or the emergency environment data include information that is likely to be consistent with a traumatic injury to the victim. In an implementation, the CSG engine 150 may continue to receive 430 the transport environment data 160 while on-site at the emergency scene 120 and during patient transport away from the emergency scene. The CSG engine may continue to load and evaluate protocols 420 with the previously received off-site data supplemented with the data received on-site.

Physiologic data and observations such as low blood pressure, fast and/or shallow breathing, cold temperature, clammy skin, weak and irregular pulse may all indicate trauma and/or shock due to trauma. Thus, one or more of these conditions may trigger an indication of trauma and need for a trauma protocol. In some cases, just an incident type or MOI may be sufficient to indicate a high likelihood of trauma (e.g., a car collision or a fall). Once a caregiver completes an initial trauma assessment on scene, other protocols may be invoked. The CSG engine 150 loads protocols such as a rapid trauma survey or a spinal motion restriction protocol automatically as the engine 150 receives the trauma assessment data.

Based on the evaluation 420 of the loaded protocols, the CSG engine 150 may identify actionable medical conditions and associated interventions. Each identified intervention is evaluated relative to interventions in progress and any other newly identified interventions to determine an order of priority and to determine the next best intervention. In other words, the CSG engine 150 tracks the care state of the patient and decides or advises the caregiver on what the immediate action or actions should be. The care state of the patient includes the physiologic state of the patient along with the overall status of the resuscitation. It answers the caregiver question, “what do I do right now?” Based on the evaluation 460, the CSG engine 150 may select 470 the at least one action item for the caregiver and/or a medical device and generate 480 at least one instruction for this action item. The CSG engine 150 may then provide 490 the instruction to the caregiver 103 via the CSG UI 155 and/or to the medical equipment 170 via the communicative coupling 199. Once the instruction is generated, the CSG engine returns to monitor 450 the physiologic and environmental data.

As an example, at the stage 410, the CSG engine 150 may determine from the emergency incident notification information 405 that there is a high likelihood of trauma due to a high-speed motorcycle collision as the MOI. The engine 150 may also receive vital sign data at the stage 440. These vital signs may indicate that the patient is tachycardic, hypotensive, has decreased O2, and increased lactate. As possible causes of these conditions in response to traumatic injury as indicated by the MOI are shock, cardiogenic shock, tension pneumothorax, and cardiac tamponade, the engine 150 may load and evaluate 420 protocols associated with these conditions. For example, the engine 150 may load an E-FAST protocol, a tension pneumothorax protocol, a cardiac tamponade protocol, and a cardiogenic shock protocol (e.g., the protocols 902, 904, 908, and 932 in FIG. 9B) to differentiate and determine therapies. The at least one action item selected at stage 470 may be a lung scan at the side of the victim's body where the victim perceives pain. At the stage 480, the CSG engine 150 may generate an instruction for the caregiver and provide the instruction along with examination procedures at the stage 490. The method 400 may continue to monitor the physiologic and environmental data at the stage 450 and receive a result of the lung scan. If that result is positive for tension pneumothorax, then at the stage 420 the method would prioritize the tension pneumothorax protocol and provide guidance. However, the guidance may vary based on environmental data. For example, the skill level or scope of practice of the caregiver as indicated by the environmental data may determine if the CSG engine 150 provides a needle decompression or chest tube instruction. If the lung scan is negative for tension pneumothorax, then the engine 150 would prioritize diagnostic steps in the cardiac tamponade protocol. If these diagnostic steps rule out cardiac tamponade, then the CSG engine 150 would proceed to evaluate the cardiogenic shock protocol.

As another example, the traumatic injury may result from a MOI identified as a fall in the emergency incident notification information received at the stage 405. The environmental data received on-site at the stage 445 may be a set of voiced observations by the caregiver that indicate an adult patient who on initial contact is unconscious, breathing rapidly (e.g., about 36 breaths per minute) and shallow with a detectible pulse of about 97 beats per minute. In an implementation, the caregiver may utilize medical equipment to measure the breathing rate and/or the heart rate and the CSG engine 150 may receive this information as physiologic data from the medical device at the stage 440. In response to this set of symptoms, the CSG engine 150 may load and evaluate one or more protocols and select action items at stage 470 of placing an oropharyngeal airway and providing assisted ventilation with a bag valve mask and about 15 liters per minute of oxygen. Further, the CSG engine 150 may generate an instruction to transport the patient to a medical facility as soon as possible (i.e., as a high priority action). As an action item for transport, the CSG engine 150 may provide an instruction to secure the patient to a backboard. Further, the CSG engine 150 may initiate a notification to the hospital that a patient is arriving and include the observed and measured patient conditions. In response to a physical exam indicating uneven breathing, broken ribs, and a broken femur as voiced data from the caregiver received by the CSG engine, the CSG engine 150 may further identify action items that include protecting ribs with a soft bulky bandage and stabilizing the femur with a splint. As continued analysis of contextual data for the patient continues at the stage 450, the caregiver may observe that the victim's skin is becoming cool and clammy, and the medical device monitor may detect an increase in pulse rate and a decrease in blood pressure. These observations and measurements may indicate that the patient may be in shock and possibly bleeding internally. Therefore, the CSG engine 150 may load and evaluate shock and internal bleeding protocols to provide an instruction to cover the victim with a blanket and to provide administration of warm saline.

As a further example, in the case of an airway obstruction caused by something as simple to fix as blood and broken teeth stuck in the airway, the CSG engine 150 may capture physiologic and environmental data at the stage 450 which reveal hypoxia, hypercarbia, and respiratory distress. The CSG engine 150 may evaluate loaded protocols at the stage 420 and select the action items of performing an airway assessment and sweeping the oropharynx for obstructing material at the stage 470. At the stage 480, the CSG engine 150 may generate an instruction for the caregiver to perform manually sweeping and, if required, suctioning the airway to allow the patient to breathe. The CSG engine 150 may provide this instruction to the caregiver at the CSG UI 155 at the stage 480. At the stage 450, the caregiver may observe the victim to determine whether the breathing improved, and the connected medical sensors may continue to analyze oxygenation and capnographic information for incoming physiologic data. The CSG engine 150 may load additional protocols and/or re-evaluate existing or additional protocols to select action items to treat the victim based on the victim's medical state.

Referring to FIGS. 8A and 8B, examples of components and data sources for the CSG engine are shown. A quantity of each component in FIGS. 8A and 8B are examples only and other quantities of each, or any, component could be used. The CSG engine 150 may include a protocol loading engine 515 and an AI modeling engine 520. The CSG engine 150 may receive inputs from contextual data sources 899.

As shown in FIG. 8B, the contextual data sources 899 may include the transport environment data sources 165, the emergency environment data sources 190, and/or the medical equipment 170. In some examples, the contextual data sources 899 also provide data outputs from the CSG engine 150. For example, the emergency environment data sources 190 may include a touchscreen 106 as shown in FIG. 3B. In an implementation, the touchscreen 106 may receive data input to the CSG engine and may provide output at a UI (e.g., the CSG UI 155). Similarly, the microphone 584 and the speaker 585 may provide input to and output from the CSG engine 150. As another example, the medical equipment may include user interfaces that provide output information from the CSG engine 150 and may also provide input to the CSG engine 150 as physiologic data 185 and/or caregiver input information provided to the user interface of a medical device.

The contextual data sources 899 may further include one or more of an EMS charting engine 591, an EMS billing engine 593, an EMS dispatch engine 592 (e.g., as part of the emergency dispatch service 130), and remote environment data sources 598. In various implementations, the CSG engine 150 may receive inputs from and/or provide outputs to these contextual data sources 899. The remote environment data sources 598 may include one or more computing devices and/or databases associated with the remote environment 320 (e.g., telemedicine support 385, cloud server 375 and/or medical records database 390). In the presence of a network connection, the processor 108 of the mobile device 110 that executes or provides the CSG engine 150 may communicatively couple to one or more of the remote environment data sources 598. The CSG engine 150 may receive information from one or more of a remotely located telemedicine provider, a remotely located dispatch service, and a remotely located medical history database.

The contextual data sources 899 provide contextual data 898 to the CSG engine 150. Thus the contextual data 898 may include the emergency environment data 195, the physiologic data 185, the transport environment data 160, the emergency event notification information 135, data from the remote environment data sources 598 (e.g., telemedicine information, medical records database information, etc.), and data from one or more of the EMS charting engine 591 and the EMS billing engine 593.

Referring again to FIG. 8A, the protocol loading engine 515, as discussed in further detail below, loads protocols into the CSG engine as a guide for response activities and parameters. The AI modeling engine 520, as discussed in further detail below, applies AI models to the inputs to the CSG engine 150 to generate actionable outputs.

Referring to FIG. 9A, an example of inputs and outputs for an AI modeling engine of a CSG engine is shown. A quantity of each component in FIG. 9A is an example only and other quantities of each, or any, component could be used.

The CSG engine 150 receives contextual data input 980 from the contextual data sources 899. Examples of the contextual data sources 899 are shown in FIG. 8B. The contextual data sources 899 may include the mobile device 110 and the mobile device 110 may provide the CSG UI 155. Therefore, contextual data 898 may be provided to the CSG engine 150 via input to the CSG UI 155. Additionally, the CSG engine 150 provides 990 caregiver instructions to one or more of the caregiver interface device(s) 960 and the caregiver may receive the instructions via the caregiver interface device(s) 960. For example, the caregiver may receive the instructions via the mobile device(s) 110, the speaker 585, the earpiece 595, a medical device user interface (e.g., the user interface 2219 in FIG. 22), etc. and combinations thereof. In various examples, a contextual data source 899 may also be a caregiver interface device 960. Thus, a same device may both input data to and receive output from the CSG engine 150. For example, the mobile device 110 may both provide contextual data to the CSG engine 150 via input to the CSG UI 155 and may provide caregiver instructions at the CSG UI 155 where those instructions are received from the CSG engine 150.

In regard to outputs, in an implementation, the CSG engine 150 may generate and an output to the CSG UI 155 in the form, for example, of instructions or prompts for the rescuers, or information for recordation and/or display. The CSG UI 155 may make the output from the CSG engine 150 available to the caregiver as a visual, audible, and/or haptic instruction.

Additionally, the CSG engine 150 may provide output to the medical equipment 170. The output may include, for example, instructions for the medical equipment 170 to perform a particular process or procedure, information for recordation and/or display at the medical device(s), instructions to record and/or display the information, requests for medical data, etc. In an implementation the instructions to perform a particular process or procedure may cause the medical device(s) to automatically perform the particular process or procedure. In such an implementation, the instructions may function as a control signal. Instructions sent to the medical equipment 170 may include instructions to perform an intervention, parameters of the intervention, and/or instructions to record information about an intervention or other determination of the CSG engine 150 (e.g., instructions to record an event marker).

Aspects of the present disclosure are also directed to allowing a user, via inputs at a CSG UI 155, to provide instructions to the medical treatment device. During treatment of a critically ill patient, rescuers in the immediate vicinity of a patient are often consumed with tending to the medical needs of the patient, whether that includes administering electric shock or ventilation via the medical treatment device, administering chest compressions, administering ventilation, or treating wounds. Additionally, user input interfaces (e.g., keypads and other buttons for inputting information) that are local to the medical treatment device can be cumbersome to operate in time-critical situations. Instead, in some examples, users at a mobile device 110 can control one or more functional operations and/or provide one or more inputs at a user-friendly, convenient touchscreen at the mobile device 110 without interfering with patient treatment. In some scenarios, the caregiver providing direct care to the patient may also operate the mobile device 110. In other scenarios, a caregiver that is providing intermittent care and/or assistance to another caregiver and not providing direct care to the patient for some period of time may operate the mobile device 110. In some examples, mobile device 110 users can input patient information, record event markers, initiate 12-lead ECG analyses, or record a device snapshot. Therefore, allowing a user to provide instructions to activate one or more operations of the medical treatment device via the mobile device 110 provides enhanced technical flexibility that is not available when operating locally at the medical treatment device by allowing supervisors or other personnel at the scene of a medical event to observe, in real-time, how the medical event is progressing without having to hover over the treatment area, which may impede patient care.

In response to receiving user inputs at the mobile device 110 associated with one of the control operations at the medical treatment device, the mobile device 110, in some implementations, transmits an instruction signal to cause the respective operation to occur at the medical treatment device. In some examples, instruction signals sent from the mobile device 110 to the medical treatment device can instruct the medical treatment device to update patient information, treatment information, or diagnostic information for the medical event. In response to receiving the respective signal, the medical treatment device performs the respective operation associated with the instruction signal, which may include storing provided information (e.g., transmitting patient information for updating at the medical treatment device) or recording an event marker (e.g., transmitting a treatment/event marker for the medical treatment device to record in the patient care record) or initiating a snapshot (e.g., transmitting an instruction signal for the medical treatment device to initiate a snapshot of ECG associated with the time of the instruction input) or activating an analysis feature (e.g., instruction signal for the medical treatment device to perform a 12-lead analysis) at the medical treatment device. In some embodiments, the instruction signals can also include control signals for causing the medical treatment device to initiate electrotherapy or apply another therapeutic treatment.

In some examples, upon initiation and/or completion of the respective operation, the medical treatment device transmits a notification signal to the mobile device 110. In response to receiving the notification signal from the medical treatment device, the mobile device 110 can cause display of a notification message at the mobile device 110 that the respective action is being performed; and then a subsequent notification signal from the medical treatment device for the mobile device 110 to display a notification message that the respective action has been performed. Thus, the systems and methods described herein provide a technical solution to the technical problem of enabling control of a medical treatment device by more than one user providing inputs at one or more mobile device 110 that are remote from the medical treatment device. For example, before the improvements described in the present disclosure were developed, personnel were limited to providing inputs or controlling operations directly at the medical treatment device interface. Because of this technical inconvenience, event markers, 12-lead analyses, and snapshots failed to be recorded at significant treatment points, reducing the effectiveness of event debriefs, personnel evaluations, and patient condition analyses. Therefore, the systems and methods described herein provide a solution to the clinical problem of providing patient care during critical medical events based on all available information in real-time (e.g., how treatment has been provided and how a patient is responding to all types of administered treatment). Further, the systems and methods described herein also solve the clinical problem of allowing supervising personnel to provide real-time treatment feedback to rescuers and others providing direct medical care, which creates a more effective clinical environment for both new and seasoned rescue teams and individuals.

In an implementation, the output from the CSG engine 150 to the medical equipment 170 may include instructions for action at the medical equipment 170. For example, the output may include, for example, an alarm instruction, an event marker instruction, a snapshot recording instruction, etc. As another example, the output may include a user selection of whether the patient is an adult, pediatric, or neonatal patient, which can be transmitted to the medical equipment 170. In some examples, the patient type may determine alarm set points, waveform scale, defibrillation energy, and/or type of case information that is relevant to and determinative of patient care. In some implementations, the output may include a request to the medical equipment 170 for any patient information already stored at the medical equipment 170.

The CSG engine 150 provides the medical device instructions 995 to the medical equipment 170 via the communicative coupling 199. In an implementation, the instructions 995 to the medical equipment 170 may include closed loop control instructions for at least one medical device. Closed loop control may also apply to multiple interventions and devices. For example, the CSG engine 150 may receive indicators of the effects of a vasopresser, ventilation, and chest compressions. The CSG engine 150 may generate and control instructions to the caregiver, the ventilation device, and possibly an automated chest compression device based on these indicators in a closed loop manner such that the received data determines the instructions without requiring separate caregiver input or control of the medical devices.

With regard to medical devices, the term closed loop control, as used herein, may refer to control of one or more operational parameters for one or more medical devices, such as with relatively little or no required user action, participation or intervention, and can include reference to, but is not limited to fully automated or fully automatically regulated control. Closed loop control may include, for example, device facilitated or algorithmically facilitated tracking, control, and adjustment of one or more parameters, which may or may not include user involvement or participation. Where user involvement or participation is included, it may include, for example, confirming a suggested or recommended medical device setting change or configuration, deciding on implementing a course of action, selecting one of several suggested courses of action, responding to a presented alert or alarm, or other decisions, choices, or actions. User involvement or participation could also include, for example, setting or changing a parameter, where a closed loop control algorithm proceeds from there, initially according to the user-set or user-changed parameter setting. In various embodiments, if there is user involvement, it may be, for example, among other things, in whole or in part user-initiated, or in whole or in part prompted, suggested, recommended, or required. In some embodiments, closed loop control may be utilized but may be subject to manual adjustment or override by the user.

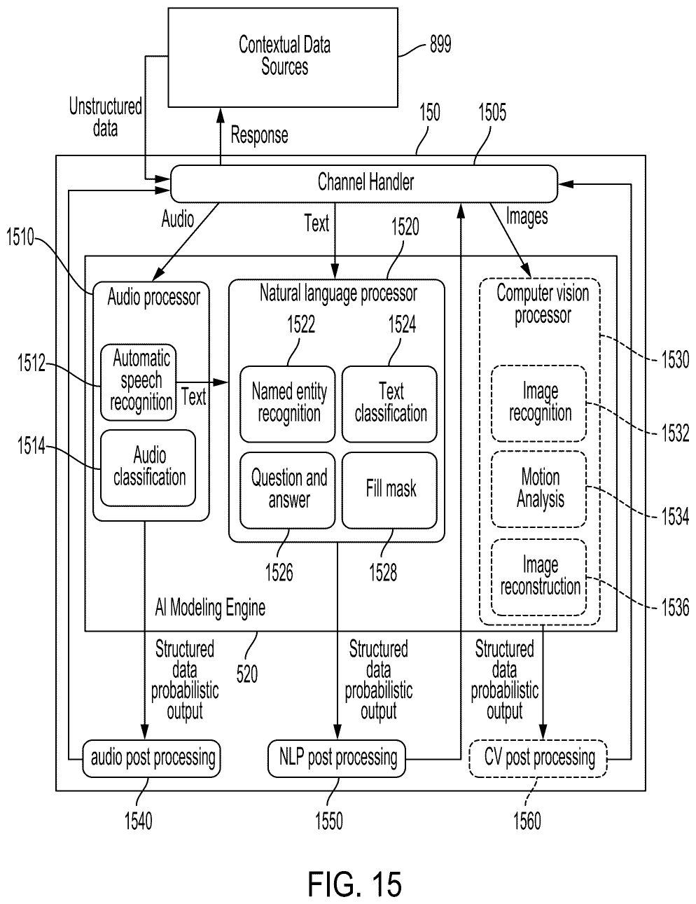

In an implementation, the CSG engine 150 includes the AI modeling engine 520. As discussed in detail in conjunction with FIGS. 15-19 below, the AI modeling engine 520 receives unstructured raw audio, text, and/or image data from speech, written materials, data files, and images. This unstructured raw data may come from audible speech, written caregiver reports, medical device data files, for example in a JavaScript Object Notation (JSON) format, or video or still images. Capturing unstructured data enables the AI modeling engine 520 to convert this data into structured and actionable data that improves patient care. The AI modeling engine 520 converts the unstructured data to structured data using AI models and feeds the structured data to the CSG engine 150 to provide caregiver guidance. The structured data conforms to data elements of a protocol. For example, the structured data may conform to data elements of a trauma protocol. The structured data is associated with a probability and the CSG engine 150 may determine and generate care instructions based on the structured data and the associated probability.

The CSG engine 150 and the AI modeling engine 520 may receive the environmental data 186 as an input 980 and the physiologic data 185 as input 985. The environmental data 980 may include voice data from the caregiver(s) and/or the victim(s). In an implementation, the environmental data 980 may also include ambient sounds from the emergency environment. In various implementations, the environmental data 980 may further include camera data, scanner data, data captured by a touchscreen, location data such as GPS data and/or cellular location data, data captured by a heads-up device, and/or dispatch data (which may include the emergency event notification information 135). In some instances, the environmental data 980 may further include information from a remotely located telemedicine provider (e.g., voice data, data files, images, etc.). For example, the verbal conversation between the telemedicine provider 389 and a local caregiver 103 may be unstructured data provided to the AI modeling engine 520 as environmental data 980. The input 985 may include data input from the one or more medical devices. In an implementation, this input may be textual input in the form of a text data file, for example, in a JSON format.

The AI modeling engine 520 receives the physiologic data and the environmental data as unstructured data and converts this unstructured data to structured data conforming to data elements associated with a patient care protocol. The AI modeling engine 520 then applies machine learning models to the structured data to select at least one action item for the caregiver and/or the medical device. The AI modeling engine 520 includes machine learning models associated with protocols (e.g., trauma protocols and/or non-trauma protocols) and the AI modeling engine 520 trains and updates the models based on the inputs 980 and 985. Thus, the machine learning models of the AI modeling engine 520 are initially trained on historic data but are then optimized, updated, and improved as use of these models in practice provide additional training data.

The environmental data 186 originates from the emergency environment data sources 190. The physiologic data 185 originates from the medical equipment 170. The environmental data 186 may also include physiologic data observed or measured by the caregiver 103 and provided to the CSG engine 150 via the mobile device 110 and/or one of the environmental data sources 190. In an implementation, the caregiver 103 may obtain this physiologic data from a device that is not communicatively coupled to the mobile device 110. The caregiver 103 may provide this physiologic data verbally (e.g., via audio input to a microphone) and/or manually (e.g., via touchscreen or heads up display). For example, if the caregiver 103 manually palpates a pulse, they might provide the pulse rate in this manner. As another example, if the caregiver 103 visually estimates a volume of blood loss, they might provide this physiologic data in this manner as well.

The CSG engine 150 provides two types of output, caregiver instructions 990 and medical device instructions 995. The CSG engine 150 provides the caregiver instructions 990 visually, audibly, and/or haptically via the CSG UI 155.

In an implementation, the instruction 990 may further include a transcript for the telemedicine provider based on the structured data output. In some cases, the CSG engine 150 may curate the transcript to emphasize particular data items associated with a confidence metric over a certain threshold. In other cases, the CSG engine 150 may select portions of the input 980 and 985 and create a report for the telemedicine provider that is relevant to the type of medical care that the telemedicine provider supports (e.g., if the telemedicine provider is providing ultrasound guidance for a suspected pneumothorax, the CSG engine 150 may curate a report that includes all information relevant to the pneumothorax inquiry and exclude information that is likely irrelevant to the pneumothorax, such as a broken leg bone or an eye injury. The transcript may enable the telemedicine provider to view portions of data that the first responder may neglect to communicate based on an assumption of low priority or because the first responder merely forgets to communicate that information. For example, even though a first responder believes that the presenting condition is a pneumothorax, the transcript may include information that indicates a cardiac tamponade with a confidence metric over the threshold but less than pneumothorax. Upon review, the telemedicine provider may recognize that the condition is in fact a cardiac tamponade and provide appropriate input to the CSG engine 150. Based on this input, the CSG engine 150 may re-direct the caregiver. In some implementations, there may be communication delay or gap with the telemedicine provider. The transcript may enable the telemedicine provider to catch up with the caregivers on scene without interruption of their work.

In an implementation, the AI modeling engine 520 may include a natural language processor (e.g., the NLP engine 1520 discussed in further detail below in regard to FIG. 15). Caregiver observations are particularly important in trauma because in many cases the effects or indicators of trauma are difficult, or in some cases impossible with current medical technology, to measure using an instrument. As similarly discussed above in regard to FIG. 2A, patient responsiveness, respiratory distress, mental confusion, visible wounds, bleeding, skin color, pupil dilation, broken bones, bruises, etc. are examples of critical information initially available most readily by caregiver observation. These observations may prompt particular physiologic parameter measurements. As examples, an observation of pallor may trigger an instruction to a medical device and/or a caregiver to measure blood pressure or lactate or an observation of cyanosis may trigger an instruction to a medical device and/or a caregiver to measure CO2, blood pH, and lactate. When assessing and treating a victim, the caregiver may intentionally speak out loud and also may speak out loud out of habit as a way of “thinking out loud” and/or communicating with the victim or other caregivers. The natural expressions and language of a caregiver are unstructured raw data that does not necessarily conform to the normalized and structured data that a machine learning algorithm can analyze. The NLP engine 1520 provides an efficient way for the CSG engine 150 to capture data that does not rely on intentional human input. In other words, the caregiver can proceed with their tasks uninhibited by data entry tasks and by merely speaking out loud regarding observations and actions, the CSG engine 150 can capture this data. Thus, the human caregiver and the NLP engine 1520 function as a non-invasive sensor that collects data for the victim based on observations of the caregiver 103.

A blood pressure measurement provides a simple example of the conversion from unstructured data to structured data and subsequent guidance. A caregiver may say out loud, “I've got a cuff reading of 90 over 70.” The AI modeling engine 520 would first convert this audible utterance to text. Then the AI modeling engine 520 would use trained models to recognize physiologic data, specifically blood pressure data. This may be due to a model trained to recognize that a number “over” another number (e.g., “90 over 70”) is a blood pressure and further that the first number is systolic pressure, and the second number is diastolic pressure. Further the trained model would recognize that “cuff” combined with “number over number” is a blood pressure cuff as part of a non-invasive blood pressure (NIBP) reading, as opposed to an invasive blood pressure reading. The AI modeling engine 520 would then generate structured data of “systolic pressure 90,” “diastolic pressure 70,” and “NIBP.” The AI modeling engine 520 would provide this structured data to the CSG engine 150. The CSG engine 150 would, in turn, use this structured data to notify the caregiver that the blood pressure is low, recognize a correlation between the low blood pressure and an elevated heart rate and respiratory rate also measured and guide the caregiver to check for hemorrhage with ultrasound.

In an implementation, the heart rate and respiratory rate in the example above may be inputs from the medical device in a JSON data file. Like the audible information, the AI modeling engine 520 may convert the JSON data file to structured data elements of heart rate and respiratory rate and provide this information to the CSG engine 150 to generate caregiver and/or medical device instructions.

As another example, based on the captured speech data “hemorrhage” as environmental data received by the CSG engine 150, the CSG engine 150 may evaluate the trauma protocols and select a tourniquet application as the at least one action item. However, if the captured speech data is “truncal hemorrhage” or a combination of “hemorrhage” with “central aorta,” then the CSG engine 150 may evaluate the trauma protocols and provide instructions for packing and/or spray foams instead of a standard pressure tourniquet. Furthermore, based on an input of “truncal hemorrhage,” then the CSG engine 150 may predict a high likelihood of bleeding and air leaks above or below the diaphragm and predictively and proactively access instructions for appropriate upcoming procedures. Additionally, based on information like “truncal hemorrhage,” the CSG engine 150 may determine a disposition location for the victim that is able to provide surgery.

In addition to converting unstructured data to structured data that is consumable by the CSG engine 150, the AI modeling engine 520 may predict caregiver workflow. In this manner, the CSG engine 150 does not merely guide a caregiver along a caregiver's chosen workflow but rather receives contextual data (e.g., the medical device inventory 805, the medical supply inventory 815, the caregiver skill record 825, and other contextual data including location and navigation data 835 and/or data available from the context utilities 599) and determines priorities for care. The CSG engine 150 then provides guidance that pushes the caregiver to an optimized state of readiness based on these priorities. The CSG engine 150 enables this optimized state of readiness by further providing information and instructions regarding the medical devices and other tools at the caregiver's disposal. The predictions of the AI modeling engine 520 based on the totality of inputs to this engine (e.g., through the contextual data sources 899) enable the CSG engine 150 to recognize connections between items of information that may elude the caregiver, particularly with the time constraints, multiple physical systems, and life-threatening injuries typical of a traumatic injury.

In an implementation, the CSG engine 150 may request additional information in order to refine instructions based on the structured output. For example, an output of “car crash” as determined by the NLP engine 1520 may trigger an audible prompt from the CSG UI 155 to ask, “what was the approximate speed at impact?” The guidance for a trauma injury due to a crash at low speed (e.g., 16-64 kph) will differ significantly from the guidance for a trauma injury due to a crash at high speed (e.g., 96-160 kph, for example. As other examples, guidance for multiple victims may differ significantly from guidance for a single victim, guidance for a pediatric, obese, geriatric, and/or pregnant victim may differ significantly from the guidance provided in the absence of these factors, guidance for a blunt trauma may differ significantly from a penetrating trauma, and guidance for a victim that is hemorrhaging and/or not talking may differ significantly from the guidance for a victim that is not bleeding and/or talking. The output from the NLP engine 1520 may correspond to key words that are associated with various protocol or algorithmic choices and the CSG engine 150 may query the caregiver or search within previously entered data to enable a decision between protocol choices.

In an implementation, the output from the NLP engine 1520 may also populate a patient chart (e.g., through the charting application 131) and thereby simplify and streamline the logistics of the response. In this case, hand off to another team or facility is more efficient and the data is more accurate than if the caregivers have to take time away from patient care to generate such charts.

In an implementation, the NLP engine 1520 may enable telemedicine support, e.g., support from the telemedicine provider 389 in FIG. 5. The telemedicine provider 389 may communicate with the emergency environment via an audio or audio/visual communications link. For example, a smartphone, tablet, laptop, workstation, or other computing device and/or mobile device may provide this communications link. In the absence of the NLP engine 1520, the caregiver 103 has to listen to the voice of the telemedicine provider 389, either through a speaker/microphone earpiece (e.g., the earpiece 595) or through the mobile device 110 or another telecommunications device. It may distract the caregiver from the patient care tasks to focus on a particular communications device and also, in a noisy emergency environment it may be difficult for the caregiver to hear the voice of the telemedicine provider 389. The NLP engine 1520 can integrate the telemedicine instructions into a single support interface provided by the CSG engine 150. For example, the device 310 providing the telemedicine communication link may provide the voice signal from the telemedicine provide as an input directly to the processor 108 of the mobile device 110. In turn, the processor 108 of the mobile device 110 may provide this as a direct input to the CSG engine 150 in addition to or in place of converting the voice signal to an audible speaker output. As such, the telemedicine communication device 310 is another example of a contextual data source 899 that may provide input to the CSG engine 150. The NLP engine 1520 may receive this voice signal input, generate a structured output, and incorporate that output into instructions provided at the CSG UI 155.

Referring to FIG. 9B, an example of a protocol loading engine is shown. A quantity of each component in FIG. 9B is an example only and other quantities of each, or any, component could be used.

The protocol loading engine 515 may load one or more protocols for use and analysis by the CSG engine 150. These protocols may include one or more of a bleeding protocol 910, an airway protocol 920, a breathing protocol 955, a circulation protocol 940, a loss of consciousness (LOC) protocol 960, a rapid trauma assessment protocol 915, a focused trauma assessment protocol 935, a head and neck protocol 905, a geriatric protocol 925, a spinal stabilization protocol 930, a cardiogenic shock protocol 932, an extremities protocol 945, an infant protocol 950, a chest protocol 965, an abdomen protocol 970, a pregnancy protocol 975, a cardiac tamponade protocol 902, a pneumothorax protocol 904, an International Life Support (ITLS) assessment protocol 906, an extended focused assessment with sonography in trauma (E-FAST) protocol 908, etc. The protocols are not limited to those shown in FIG. 9B and may include one or more other or additional care protocols as indicated by the “XYZ” protocol 900. Each protocol includes care activities and parameters as elements of standardized care and/or those customized by a medical director. In an implementation, the protocol loading engine 515 may unload or disable a particular protocol. For example, for a female confirmed not to be pregnant or a male, the engine 515 may disable the pregnancy protocol 975. For a child, the engine 515 may disable the geriatric protocol 925 and, similarly, for an elderly individual, the engine 515 may disable the infant protocol 950. In an implementation, the protocol loading engine 515 may load protocols, or exclude protocols, based on the exclusion/inclusion criteria discussed in regard to FIG. 3C-2.

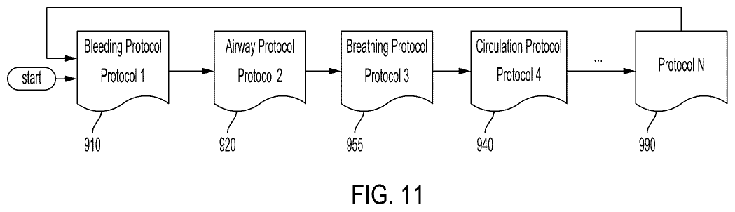

Referring to FIG. 10, a schematic diagram of parallel protocol processing is shown. A quantity of each component in FIG. 10 is an example only and other quantities of each, or any, component could be used. Parallel processing is of particular importance in a traumatic injury because the multiple physical systems that are typically injured in trauma cause multiple and simultaneously occurring life-threatening medical conditions. For example, an injury to the chest may causes hemorrhaging and respiratory distress (e.g., due to lung damage) or may further include cardiac arrest that develops because of the respiratory distress. As another example, a trauma may lead to shock, but the shock may manifest itself over the course of care and not be present upon initial observation of the victim. As a further example, in the case of an extremity trauma without any other injury (e.g., a broken leg without any other injuries), the priority of care is to address the extremity trauma. However, in the case of a multi-system trauma (e.g., a broken leg along with head trauma), the CSG engine may evaluate airway, breathing, and circulation along with the extremity trauma in the absence of bleeding or evaluate bleeding, airway, breathing, and circulation in the presence of bleeding along with the extremity trauma. In these cases, evaluation of protocols in parallel may be particularly critical because effective treatment of the victim may require steps from various protocols to be interspersed with one another rather than each protocol in a series of protocols being executed from start to finish, for example as shown in FIG. 11. For example, a victim may present with both an airway obstruction and respiratory distress from a pneumothorax. If the caregiver focuses on the airway obstruction without intervening for the pneumothorax, the victim may die even if the caregiver clears the airway obstruction. As another example, a trauma victim may present with intra-abdominal bleeding from a spleen, extremity hemorrhage from an extremity amputation (e.g., a traumatic injury in the form of a severed limb), high blood alcohol content, and an opioid overdose. Such an example provides competing factors that if treated in the wrong order may lead to a victim's death. For example, a focus on the head injury without an intervention directed at the splenic bleeding and/or a focus on the extremity amputation without an intervention for the airway obstruction could both lead to a deadly outcome for the victim. For both of the above examples, it is also possible for a victim to have suffered burn injuries. These examples demonstrate the complex nature of trauma and many critical factors involved in providing care to a victim or patient, such as a trauma victim.

As an example of the variety of data that the CSG engine 150 may receive for a victim, user observations and medical device data may implicate airway, bleeding, and loss of consciousness protocols. For example, an airway issue may be associated with caregiver observations of “foreign body obstruction” and “gurgling” as verbal inputs and medical device data indicative of hypoxia (e.g., SpO2 and EtCO2 data) along with chest velocity and force measurements from a pneumograph and telemetry data indicative of tachycardia. At the same time, physiologic data for heart rate, blood pressure, lactate concentration, and oxygen perfusion may indicate bleeding. Human observations for bleeding may include a location of an injury (e.g., chest, abdomen, or retroperineum), an observation of blood on the ground or another surface beneath the victim, and an observation of broken bones. Ultrasound data may further narrow the location of an internal bleed or hemorrhage. Bleeding may be missed by a first responder (e.g., if it is dark, if the bleeding is under the victim and not visible by the caregiver, or if there is internal bleeding). Thus, monitoring indicators of bleeding in parallel with airway parameters may detect bleeding sooner than a human caregiver would detect the bleeding. The CSG engine 150 may raise the priority of a bleeding intervention based on the data. In the case of a victim suffering from loss of airway and bleeding, there may be a loss of consciousness (LOC). LOC is generally of a lesser priority in a sequential care plan traditionally executed by a human caregiver. However, the ability to detect indicators of impending LOC and potentially provide interventions earlier may improve outcomes for emergency medical event victims, including trauma victims. As an example of inputs related to LOC, the medical devices may provide heart rate, blood pressure, lactate concentration and oxygen data. The human caregiver may verbally input a Glasgow coma scale rating, a victim movement assessment, a pupil dilation assessment, and an assessment of the communication capability of the trauma victim (e.g., is the victim speaking). The CSG engine 150 may also record victim speech as an indicator separate from the caregiver assessment.

It would not be unusual for all of the data and observations listed above to be received by the CSG engine 150 and analyzed to determine a next best step or series of steps for the caregiver and medical devices. If the emergency scene 120 is in close proximity to a hospital and the heart rate, blood pressure, lactate concentration, and oxygen are within a normal range or close to a normal range, the next best step may be transport. If the victim is unconscious, a protocol may indicate intubation but if the victim's oxygen levels are adequate, then a fast drive to the hospital may be the next best step rather than intubation. In these examples, a location input may be critical to the decision making by the CSG engine 150 as far as the determination of next best step. Additionally, for something like intubation, for example, caregiver skill level is critical because not every practitioner is qualified to perform an intubation. Thus, availability of telemedicine to provide physician support may also be critical.

The CSG engine 150 evaluates multiple protocols, 910, 920, 955, 940, 990 without a pre-determined sequence assigned to the protocols and without requiring a first protocol to be complete before beginning a second protocol. For example, unlike sequential protocol processing as illustrated in FIG. 11, the CSG engine 150 does not predetermine a sequence like, for example, the traditional bleeding-airway-breathing-circulation sequence of trauma care. Rather, the CSG engine 150 can apply the bleeding, airway, breathing, and circulation protocols in parallel. The analysis of parameters associated with these protocols does not need to happen in sequence and in fact indicators of conditions associated with multiple protocols may be simultaneously present.

For example, if an initial assessment of the patient indicates a traumatic injury to the chest, like a broken rib, the CSG engine 150 may determine a probability associated with injury invoking each of the bleeding, airway, breathing, and circulation protocols. The highest probabilities from the broken rib may be bleeding and breathing and these may be approximately equal. The CSG engine 150 can then prepare instructions for addressing both of these situations. The CSG engine 150 may provide a caregiver instruction to address the bleeding and essentially simultaneously instruct a ventilator to power on and provide operational settings so that the ventilator is ready to go as soon as the caregiver can attach it to the patient. Further, the CSG engine 150 can monitor the physiologic indicators for airway and circulation issues without impeding progress on the bleeding and breathing interventions. Over the course of treatment, there may be changes in the physiologic indicators that may indicate that two competing life-threatening conditions have changed priority in terms of which is likely to kill the victim sooner. As an example, an airway issue may be of higher priority than a circulation issue but as the trauma intervention proceeds, the circulation issue may supersede the airway issue, for example, if the airway is partially blocked but the victim goes into cardiac arrest. The first responder may be able to circumvent the airway blockage but without immediate defibrillation, the cardiac arrest may kill or severely damage the victim before the partial airway blockage causes death or permanent damage.

In contrast to the CSG engine 150, a human caregiver can only do activities, like those exemplified above, one at a time and in sequence. Moreover, first responders and physicians are specifically and intentionally trained to do these activities in sequence to avoid errors. In general, a human practitioner focuses on a subset of information at each step of care provision and temporarily disregards other information in order to function effectively and in a hierarchical manner. In accordance with advantages of embodiments described herein, the CSG engine 150 can simultaneously store and analyze all of the available information including past and present data at once where individual humans may be incapable of such an overly complex analysis. The CSG engine 150 can simultaneously analyze, prioritize, sort, and monitor all information available while a single individual can only handle a limited set of decision-making processes at once. Synthesizing and filtering the vast amounts of information into actionable tasks and recalling clinical protocol steps becomes overwhelming for a human care provider, especially considering competing demands on cognitive load such as noise, light, stress, hunger, tiredness, environmental distractions, driver busy maintaining control, inability to access and assess patient, physical demands, among others. In general, human practitioners typically exhibit task fixation, where they focus only on a smaller subset of total information potentially available at each step of care provision and temporarily disregard other information in order to function effectively and in a hierarchical manner. The CSG engine 150 can hold all of the information at once without the need to focus that a human brain requires.

As another example of a benefit of parallel processing, the CSG engine 150 can look at a distribution of care activities based on probabilities and prepare information and instructions according to non-zero probabilities. For example, a first responder may believe that the victim has suffered a pneumothorax. Based on the inputs from the emergency environment interface device(s) 190 and the medical equipment 170, the NLP engine 1520 may determine structured output of pneumothorax and cardiac tamponade, with a probability of 60% for the pneumothorax and 40% for the cardiac tamponade. The CSG engine 150 may then prepare instructions for both situations. Furthermore, as the system receives additional information, the probabilities may change to indicate a higher likelihood of cardiac tamponade and the CSG engine 150 may provide an alert or early warning and guidance for the human caregiver to change course (e.g., as shown in FIG. 14D-1).

As a further aid to parallel processing, the CSG engine 150 and the NLP engine 1520 can receive and incorporate caregiver observations and patient history through verbal input from the caregiver. For example, a first responder arriving on an emergency scene may interview the victim, read or scan a medical alert bracelet or medication container, and/or speak with a family member. With the benefit of this medical history information, the CSG engine 150 can evaluate multiple protocols for similarly presenting conditions and differentiate based on the provided history.

In an implementation, the CSG algorithm 151 of the CSG engine 150 may implement non-linear evaluation methods to evaluate physiologic parameters, evaluate and load protocols, and/or to identify a recommended clinical intervention. The non-linear evaluation of multiple physiologic parameters may function as a patient condition estimator where the estimated patient condition corresponds to a particular intervention.

One example of a patient condition estimator is a Kalman filter. Generally, the Kalman filter models a linear system with Gaussian distribution, which may not be encountered in the physiologic setting. Thus at least one example of the patient condition estimator executes an extended Kalman filter (EKF) to solve the problem of non-Gaussian, nonlinear filtering. The EKF is based upon the principle of linearizing the measurements and evolution models using Taylor series expansions. The series approximations in the EKF process may, however, lead to poor representations of the nonlinear functions and probability distributions of interest. As a result, the EKF may diverge. Therefore, at least one example of the patient condition estimator executes an unscented Kalman filter (UKF) based on the hypothesis that it is easier to approximate a Gaussian distribution than it is to approximate arbitrary nonlinear functions. It has been shown that the UKF leads to more accurate results than the EKF and that it generates much better estimates of the covariance of the states (the EKF often seems to underestimate this quantity). The UKF, however, has a limitation in that it does not apply to general non-Gaussian distributions, as is often the case with ECG spectral distributions.

Another example of the patient condition estimator is a Sequential Monte Carlo process, also known as a particle filter. This example overcomes the non-Gaussian limitations and allows for a complete representation of the posterior distribution of the states, so that any statistical estimates, such as the mean, modes, kurtosis, and variance, may be easily computed. Particle filters may, therefore, deal with any nonlinearities or distributions. Particle filters rely on importance sampling and, as a result, require the design of proposal distributions that can approximate the posterior distribution reasonably well. In general, it is hard to design such proposals. The most common strategy is to sample from the probabilistic model of the state evolution (transition prior). This strategy, however, may fail if the new measurements appear in the tail of the prior or if the likelihood is too peaked in comparison to the prior.

Some other example patient condition estimators include an estimator/predictor trajectory tracking technique known as the Unscented Particle Filter (UPF) and other non-linear estimators such as, for example, non-linear regression, neural networks, and convolutional neural networks.

In some embodiments, the CSG algorithm 151 of the CSG engine 150 may be based, at least in part, on a Hidden Markov Model (HMM) with the sequence of patient condition and therapeutic intervention vectors as the input. A state transition matrix can be developed using HMM techniques known to those skilled in the art. In particular, the sequence of underlying patient states and recommended therapeutic interventions primitives, and condition and resultant intervention sentences is modeled as a hidden Markov model (HMM), defined as a variant of a finite state machine having a set of states, Q, an output alphabet, O, transition probabilities, A, output probabilities, B, and initial state probabilities, II. The current state is not observable. Instead, each state produces an output with a certain probability (B). Usually the states, Q, and outputs, O, are understood, so an HMM is said to be a triple, λ=(A, B, II). Each value of output alphabet, O, can be given a unique threshold and coefficient set. For instance, the HMM will have the transition probabilities stored in it, as a result either of training against a database or patient-specific training, indicating such understood “facts” (i.e., therapeutic intervention grammar). For instance, that it is a very low probability that a patient's blood pressure will decrease by more than 20 mmHg if they were just given a dose of a vasopressor: there must been either an intervening the blood pressure reading may be lost in noise, or, alternatively, blood pressure may decrease with sepsis as a more likely estimate of the patient's underlying condition, in which case the optimal therapeutic intervention would be to continue vasopressor but also give broad-spectrum antibiotic for potential septic infection, and perform point of care blood analysis to do a measure of lactic acid.

Other techniques known in the field of HMM classification may be employed. For instance, discriminative training techniques that dispense with a purely statistical approach to HMM parameter estimation and instead optimize some classification-related measure of the training data. Some examples include maximum mutual information (MMI), minimum classification error (MCE) and minimum phone error (MPE) and use of the Viterbi algorithm to find the best path. Other techniques are to keep a set of good candidates instead of just keeping the best candidate, and to use a better scoring function (re scoring) to rate these good candidates. The set of candidates can be kept either as a list (the N-best list approach) or as a subset of the models (a lattice). Re-scoring may be done to minimize the Bayesian risk.

In some embodiments, so-called Deep Learning Networks may be employed. Deep networks may be employed for unsupervised or generative learning to capture high-order correlation of observed or visible data for pattern analysis or synthesis purposes when no information about target class labels is available. Additionally, deep networks may be used for supervised learning to directly provide discriminative power for pattern classification purposes, often by characterizing the posterior distributions of classes conditioned on the visible data. Further, hybrid deep networks may be employed where the goal is discrimination that is assisted with the outcomes of generative or unsupervised deep networks. Other classification methods include, for example, Deep Neural Networks (DNN) and Recurrent Neural Networks (RNN), Convolutional Neural Networks (CNN), Restricted Boltzman Machine (RBM), Deep Belief Network (DBN), Deep Boltzman Machine (DBM), etc.

Referring to FIG. 12, a parallel protocol method for a CSG engine is shown. The method 1200 is, however, an example only and not limiting. The method 1200 can be altered, e.g., by having stages added, removed, rearranged, combined, and/or performed concurrently.

At the stage 1205, the CSG engine 150 receives and evaluates contextual data 898. The contextual data includes physiologic contextual data from the medical devices. At the stage 1215, the CSG engine 150 loads and evaluates protocols in parallel, as described in regard to FIG. 10 above. For example, if the contextual data input to the CSG engine 150 indicates a chest trauma injury with external bleeding to an eighty-year-old male, then the system may load at least the bleeding protocol 910, the chest protocol 965, the cardiac tamponade protocol 902, the pneumothorax protocol 904, and the geriatric protocol 925. Each of these protocols may include an ordered series of steps to perform according to a standard of care and/or a preference of a medical director.

The CSG engine 150 determines a set of next possible steps at the stage 1220. The CSG engine 150 may identify multiple possible next steps from a current step based on the totality of the protocols evaluated. The CSG engine 150 evaluates the protocols based on the contextual data including the physiologic data input from the medical equipment 170. As a simplified example, the set of next possible steps at the stage 1220 may include 4 steps, with each step being the first step on each of the four protocols. The CSG engine 150 may then select one of these steps at the stage 1225 as the first next best step. Further, the CSG engine 150 may determine an order of performance for all of the next possible steps on the four protocols and may provide instructions for the caregiver and/or the medical devices based on this determined order of performance of steps subsequent to the next best step. For each identified next step for patient care, there is a protocol that corresponds to the care associated with that next step. For example, if the possible next steps are to apply a tourniquet and administer chest compressions, one trauma protocol would correspond to caregiving activities for the tourniquet application and another trauma protocol would correspond to caregiving activities for the chest compressions. The CSG engine 150 then determines, from the multiple possible next steps, at least one next best step. The next best step is the next possible step that provides the highest likelihood of improving a patient's conditions and the lowest likelihood of diminishing a patient's condition. For example, if the wound requiring the tourniquet is such that chest compressions would increase blood loss through that wound to a degree that would threaten the patient's life more than the delay in chest compressions, then the CSG engine 150 may identify the next best step as the tourniquet application. The CSG engine 150 may then select an action item based on this next best step, such as removing the tourniquet from the trauma kit 665.

The CSG engine 150 may determine the set of next possible steps based on the protocols. The engine 150 may set these priorities based, for example, on the relative probability or likelihood of a particular condition and the actionable information available to the engine 150. In an implementation, the priority of care that determines the next best step is established in a standard of care and the CSG engine 150 may follow those priorities when evaluating the various relevant clinical protocols. In an implementation, a database may include one or more of clinical expertise and/or prioritization from a medical director and the CSG engine 150 may establish priorities based on the database. The medical director's prioritization may be accomplished via pre-determined settings and/or a prioritization algorithm with weighting factors and parameters or metrics that are adjustable by a medical director. Some of these parameters may include lethality, frequency, quality of life impact, risk etc., from a library of clinical trauma conditions. In an implementation, these parameters may additionally include parameters such as demographics (age, sex, ethnicity, etc.), medical history, etc. and/or environmental factors (e.g., time to hospital, mode of transportation, available medical equipment, caregiver availability, location of trauma event, number of victims, safety of the trauma scene, etc.). In a further implementation, the parameters may include protocol step-specific metrics such as speed of intervention, ease of intervention, supplies needed for intervention, type of intervention (e.g., therapeutic or diagnostic), etc. The medical device availability may take into account the availability of a particular medical device or associated supply, the ability of the caregiver to utilize the particular medical device based on caregiver training, the availability of telemedicine support, and/or the availability of any medical supplies associated with a particular medical device. The caregiver availability may take into account the number of caregivers, the caregiver training and scope of care, and/or the priority associated with each task for the caregiver. As an additional factor, in some implementations, the CSG engine 150 may base priority on a travel distance and/or travel time to a hospital or longer-term care facility. In some cases, if the hospital is very close by, a condition may not be life-threatening in that time frame and the overall quality of care for the patient may be better if the hospital initiates treatment for that condition.

The CSG engine 150 may evaluate one or more of these metrics via a prioritization algorithm that that may include assigned or adjustable weights for the various parameters. Thus, the prioritization of care activities by the CSG engine 150 may be evidence based rather than merely protocol driven. In an implementation, the CSG engine 150 may progressively adjust the priority of care determination over time. For example, the CSG engine 150 may initially implement a protocol driven system and start to populate the database with the clinical expertise and outcomes. As the database contents grow, the CSG engine 150 may develop and adjust the prioritization algorithm. Medical directors may also adjust parameters and weights and overall system performance over time.

In some cases, a traumatic injury may present multiple life-threatening medical conditions simultaneously. For example, a situation may arise where an injury to the neck causes hemorrhaging and crushes the trachea. In these cases, the guidance from the CSG engine 150 may be particularly critical because effective treatment of the victim may require the caregiver to adjust their interventions based on nuanced changes in the victim's condition because two competing life-threatening conditions have changed priority in terms of which is likely to kill the victim sooner.

As discussed above, the CSG engine 150 may receive at least a portion of contextual data prior to arrival at the emergency scene. For example, the CSG engine 150 may receive the emergency event notification information 135 and the transport environment data 160. This off-site contextual data enables the CSG engine 150 to select and evaluate a first group of protocols prior to arrival at the emergency scene. In this manner, the CSG engine 150 can prepare caregivers and medical equipment for activities and deployment immediately upon arrival at the emergency scene. Once on-site at the emergency scene, the CSG engine 150 may additionally receive physiologic data 185 from the medical equipment 170 when the caregiver 103 couples at least one of the medical equipment 170 to the victim 101. The CSG engine 150 may further receive emergency environment data 195. Upon receipt of the physiologic data 185, the CSG engine 150 may add additional protocols and/or replace one or more protocols to form a second group of protocols to evaluate. Furthermore, as treatment progresses at the emergency scene and/or during victim transport from the emergency scene to a hospital, the CSG engine 150 may continue to add and/or replace protocols as dictated by the evolving medical status of the victim.

At the stage 1207, CSG engine 150 may evaluate, or re-evaluate, the contextual at the stage 1207. Although the method 1200 shows the evaluation of the data at discrete intervals corresponding to the stages 1205, 1207, and 1209 for illustrative purposes, in practice the CSG engine 150 receives, monitors, analyzes, and evaluates contextual data in real-time as the contextual data becomes available to the engine 150. In this manner, the CSG engine 150 can dynamically adjust guidance according to the evolving state of the victim.

At the stage 1290, the AI modeling engine 520 may determine whether a patient care state has changed. For example, if the next best step was to apply a tourniquet but then the victim experiences acute respiratory distress (RD), the RD may not be a result of the tourniquet application. The patient care state may also improve in response to the next best step or in response to a step prior to the next best step or based on the natural resolution of a victim condition. Based on the contextual data, the AI modeling engine 520 may recognize an improvement or degradation in patient condition as a patient care state change.

As another example, the patient care state may change as a result of a change in weather (e.g., a temperature drop poses additional risks to the victim and affects the necessary care) or a change in available caregiver skill and/or medical equipment (e.g., a second crew arrives with a paramedic where the first crew had only BLS training or a second crew arrives with an ultrasound imaging device where the first crew only had a stethoscope). As yet another example, a new caregiver may change the patient care state. For example, if first responders only had BLS training and a new caregiver with ALS training arrives on scene, then the available interventions may change. Similarly, establishing a connection with a telemedicine provider may represent a change in the patient care state. The patient care state may change due to a worsening or improving condition of a victim's physiologic condition. This may occur in response to the next best step or may occur independently just because the passage of time has caused another medical issue to manifest itself. There are at least several other examples of changes to the patient care state. The victim may present with burns, but a bleed may develop during the course of care. Thus, the state changes from “burn” to “burn+bleed” and the priority of care may change if the bleed presents a more imminent threat to the victim than the bleed. The victim may initially present with bodily injuries such as cuts and scrapes. However, over the course of care, the victim may become restless or agitated due to hypoxia and/or hypoglycemia resulting from a head trauma that the CSG engine 150 missed during the initial assessment. Here, the priority of care may shift from treating cuts and scrapes to checking the airway, assessing glucose levels, and treating airway and glucose conditions prior to the cuts and scrapes. Most trauma victims are at risk of shock and therefore the patient care state could change to include and/or prioritize shock if and when symptoms of shock manifest themselves. However, even shock may not take priority over bleeding and therefore bleeding at any time may also change the patient care state.

In response to a patient care state change, the method 1200 returns to the stage 1215 to evaluate protocols, which may include loading additional protocols, replacing protocols, or removing protocols, and determining the set of next possible steps 1220 and a first next best step 1225. In response to an absence of a patient care state change, the method 1200 proceeds to the second next best step 1230 according to the previously determined order of performance. The sequence of identifying next best steps repeats (e.g., with another evaluation of contextual data at the stage 1209 and another state change evaluation 1295) for N iterations until a next best step N 1235 which may be the transfer of the victim to a victim disposition site like a hospital.

In re-evaluating the protocols at the stage 1215 following a patient care state change, the CSG engine 150 may identify an updated set of next possible steps at the stage 1220 to replace a previously identified set of next possible steps. Further, the CSG engine 150 may select a new, or updated, next best step from this updated set of steps. The CSG engine 150 may select the at least one action item for the caregiver and/or the medical devices based on this updated next best step.

In order to move through the stages of FIG. 12, the AI modeling engine 520 evaluates a present medical state of the victim as reflected in the present state of all of the inputs to the CSG engine 150 (e.g., contextual data 898 from the contextual data sources 899). Based on the array of information received from the contextual data sources 899, the CSG engine 150 implements the AI modeling engine 520 may to identify a patient care state and select a next best step based on that state. For example, the trauma care state may be cardiac arrest with minimal external bleeding or may be cardiac arrest with severe external bleeding. The order of priority of activities for these two states may be different in regard to when to initiate chest compressions. Further, in the latter state, if a tourniquet is applied and chest compressions commenced, the victim state may change if the tourniquet becomes loose or another wound or internal bleeding is recognized. Thus, the AI modeling engine 520 may have to re-shuffle priorities between a bleeding protocol and a cardiac arrest protocol based on the state of the victim.

In an implementation, the AI modeling engine 520 may evaluate priorities of data with regard to the actionable nature of the data. For example, an input of “severe blood loss” may have a higher priority for immediate treatment than “victim is confused.” As another example, a drop in heart rate from 70 to 30 bpm may have a higher priority for immediate treatment than a change in a pulse oximetry value from 94% to 90%. However, although a data item may be of lower priority for treatment at one stage of care in light of the information available to the CSG system at that stage, the same data item may be of a higher priority in light of additional information. For example, an early observation of “victim is confused” may increase in priority for treatment when combined with a later observation of “face drooping on one side.” The AI modeling engine 520 keeps track of these data items and continues to reevaluate data in combination with new data. Thus, where an early observation of “victim is confused” may not produce an action item, this observation combined with “face drooping on one side” may indicate a high probability of a stroke and generate appropriate action items for stroke interventions and treatment. For example, the combination of first data item (e.g., “victim is confused”) at a first trauma state with a later occurring second data item at a second trauma state (e.g., “face drooping on one side”) may indicate a state change to a third trauma state that includes care for a possible stroke. As another example, a first trauma state of geriatric patient combined with a second trauma state of systolic blood pressure <110 may indicate a state change to shock. As yet another example, a first trauma state of cold and clammy skin combined with a second trauma state of one or more of low systolic blood pressure, anxiety, or tachycardia may indicate a state change to cardiogenic shock.

In addition to tracking data and re-evaluating next best steps as the victim care progresses, the AI modeling engine 520 may also identify and track one or more unperformed steps from the set of next possible steps. These steps may be unperformed due to a replacement of the set by an updated set of next possible steps following a state change. These unperformed steps may be necessary steps but the priority for treatment of these steps may change as the patient care state evolves. The AI modeling engine 520 may track and log these steps and then generate reminders at a later time during the patient care for these steps. The CSG engine 150 may provide these reminders to the caregiver and/or the medical devices. For example, it may be necessary to assess the neck of a victim prior to moving or transporting the victim. However, this assessment may be of lower priority while the trauma victim is losing blood. Thus, even if a spinal injury is suspected, the system may log a missed step of assessing the neck and then generate a reminder once the hemorrhage has been stopped or minimized.

In an implementation, the AI modeling engine 520 may additionally manage the evaluation of unstructured text by searching for particular items within that text rather than or prior to processing the entirety of that text by the AI modeling engine 520. The AI modeling engine 520 may include models trained to recognize a context of the patient (e.g., cardiac arrest, drug overdose, head trauma, chest injuries, etc.) and search for unstructured text particular to that context. As another, the AI modeling engine 520 may examine the unstructured text according to international classification of diseases (ICD) codes in order to facilitate charting and billing.

In addition to caregiver and medical device instructions for a next best step, in various implementations, the output from the CSG engine 150 may include a diagnosis or potential diagnosis. For example, the CSG engine 150 may evaluate the contextual data and, based on this data, identify a potential diagnosis of a victim condition along with a probability associated with the potential diagnosis. The CSG engine 150 may determine the potential diagnosis to be a diagnosis if the probability exceeds a particular threshold (e.g., 90-100%). In selecting a next best step as the at least one action item for the caregiver and/or the medical device, the CSG engine 150 may select the at least one action item based on the potential diagnosis and the associated probability in addition to the environmental and physiologic data.

As a further capability in selecting a next best step, the CSG engine 150 may take into account location and navigation information. For example, the CSG engine 150 may determine a location disposition for the victim to a location outside of the emergency environment, such as a hospital. The CSG engine 150 may identify this disposition location based on the contextual data. For example, the CSG engine 150 may determine the disposition location based on one or more of presenting condition(s) of the victim, a severity of the condition, a distance to a hospital, a travel time based on traffic and/or weather conditions, and insurance information for the victim. Based on the identified disposition location, the CSG engine 150 may select the at least one action item for the caregiver and/or the medical device. For example, if the hospital is closer and specializes in treatment of the presenting condition(s), and there is a low skill level of the EMS crew without specialized medical equipment in the transport environment, the CSG engine 150 may modify instructions for less intervention in the mobile environment. The goal in this case may be merely to stabilize and transport immediately. On the other hand, if a hospital is further away and there is some specialized equipment in the transport environment and an available telemedicine link, the CSG engine 150 may modify instructions for more intervention in the mobile environment.

Referring to FIG. 13, an example of a method of CSG is shown. The method 1200 is, however, an example only and not limiting. The method 1300 can be altered, e.g., by having stages added, removed, rearranged, combined, and/or performed concurrently. The method 1300 provides an example of an implementation of the method 1200 for a trauma victim presenting with chest trauma.

At the stage 1305, similarly to the stage 1205 in FIG. 12, the CSG engine 150 receives and evaluates contextual data. The CSG engine 150 identifies two possible conditions resulting from trauma, namely cardiac tamponade and a pneumothorax. The CSG engine 150 may identify other conditions as well, but these two conditions may each be associated with a confidence metric that exceeds a threshold. At the stage 1315, similarly to the stage 1215 in FIG. 12, the CSG engine 150 may evaluate protocols loaded by the protocol loading engine 515 based on the contextual data. These protocols may include the cardiac tamponade protocol 902 and the pneumothorax protocol 904. Additionally, the protocols may include the chest protocol 965, along with the airway, breathing, circulation, and bleeding protocols (e.g., protocols 920, 955, 940, and 910) along with protocols indicated by the inputs to the CSG engine 150. For example, the victim may be a pregnant female thus invoking the pregnancy protocol 975. There also may be an injury independent from the chest trauma, like head trauma, thus invoking the head and neck protocol 905. Although the method 1300 focuses on the cardiac tamponade and pneumothorax protocols for simplicity, the CSG engine 150 may continue to evaluate all of the other loaded protocols and if care for another condition becomes higher priority than a care step in the method 1300, the CSG engine 150 may divert from the method 1300 to rearrange the care steps to accommodate the higher priority care step. The plurality of protocols enables the CSG engine to determine a plurality of next possible steps and a next best step 1 as shown at the stages 1220 and 1225 in FIG. 12. For example, the plurality of next possible steps may include all the diagnostic steps for pneumothorax and cardiac tamponade along with steps corresponding to pregnancy, head injury, and breathing. In an example, the victim may also present with hypoxia and the next best step (e.g., corresponding to the stage 1225) may be to provide oxygen as an intervention for the hypoxia. The CSG engine 150 may then update an evaluation of the physiologic data and environmental data (e.g., corresponding to the stage 1207). If the patient now presents with tachycardia, this may represent a patient care state change at the stage 1290. Thus, the CSG engine 150 may re-evaluate protocols (e.g., at the stage 1215 or 1315) to add protocol items for tachycardia to the plurality of next possible steps. However, if the victim does not present with tachycardia, then the patient care state may be unchanged and the CSG engine may proceed with the next best step 2 (e.g., at the stage 1230 of FIG. 12). This next best step 2 may correspond to the inventory and skill inquiry at the stage 1318 to select stethoscope or ultrasound examination based on the possible pneumothorax or cardiac tamponade.

At the stage 1318, as part of the protocol evaluations, the CSG engine 150 may look for a medical device inventory 805, a medical supply inventory 815 and/or a caregiver skill record 825. The example of method 1300 assumes that a caregiver skill level allows use of ultrasound and/or that telemedicine support for an ultrasound examination is available. If the inventory information is available, the CSG engine 150 may select ultrasound as a preferred examination tool at the stage 1320. If the inventory is not available, the CSG engine 150 may query the user, for example at the CSG UI 155, to provide inventory and/or skill information at the stage 1325. Based on the query response, the CSG engine 150 may select ultrasound as a preferred examination tool at the stage 1330. At the stage 1335, the CSG engine 150 may confirm a type of examination as a stethoscope or an ultrasound examination. The CSG engine 150 may confirm the type of examination via a user query at the CSG UI 155 (e.g., via the “confirm” control 1455) and/or may receive an automated confirmation from the medical device, for example, the ultrasound equipment.

Based on the type of examination confirmed, the CSG engine may provide guidance for a stethoscope examination at the stage 1340 or guidance for an ultrasound examination at the stage 1345. The CSG UI 155 may provide medical device instruction 1480 for one of the stethoscope or the ultrasound equipment. This guidance may follow the user-controlled granularity guidance exemplified in FIGS. 14B-1, 14B-2, and 14B-3 as discussed below. For example, for either examination type, the CSG engine 150 may break down the guidance into nested guidance steps and the caregiver may control and select the level of detail based on their own experience and expertise and/or the availability of a telemedicine resource.

At the stage 1350, the CSG engine 150 may receive the results of the diagnostic inquiry by stethoscope or ultrasound. The CSG engine 150 may receive these results as caregiver observations (e.g., entered at the touchscreen 106 for the CSG UI 155 and/or entered as verbal input processed by the AI modeling engine 520. The CSG engine 150 may analyze the diagnostic results at the stage 1355 to determine if the results more likely correspond to pneumothorax (i.e., the results A listed at the stage 1360) or to cardiac tamponade (i.e., the results B listed at the stage 1370).

Additionally, or alternatively, the CSG engine 150 may receive physiologic data, for example, a recording from an electronic digital stethoscope or an ultrasound image and/or ultrasound imaging analytics from the ultrasound device. For example, the stethoscope results 1363 may indicate unequal breath sounds and hyperresonance associated with a pneumothorax. Alternatively, the stethoscope results 1373 may indicate equal breath sounds and muffled heart sounds associated with cardiac tamponade. In some cases, the stethoscope results selected (A or B) are those with the highest probability if the results are not absolutely conclusive. In the case of the ultrasound exam, the CSG engine 150 may independently (e.g., based on unsupervised analysis by the computer vision engine 1530 shown in FIG. 15) or jointly with the caregiver (e.g., based on supervised CV engine analysis and/or by presenting the caregiver with the ultrasound image for caregiver and/or telemedicine interpretation, in some cases aided by sample images corresponding to cardiac tamponade or pneumothorax) classify the ultrasound image. The ultrasound results 1366 may include the pneumothorax image 1369 or the ultrasound results 1376 may include the cardiac tamponade image 1379. The CSG engine 150 may then apply diagnostic differentiators to select the most likely condition as pneumothorax 1380 or as cardiac tamponade 1390. The diagnostic differentiators may be differentiators within results A or results B and/or differentiators included in other physiologic or environmental data. For example, an observation of jugular venous distention may support the diagnosis cardiac tamponade or pneumothorax, as opposed to another condition associated with chest injury. Further, the caregiver and/or the medical devices may provide additional data that raises the probability of one of pneumothorax or cardiac tamponade over the other when taken in combination with other data (e.g., presence of coughing, cool extremities, blue lips, discomfort that is relieved in certain positions, blood pressure changes, etc.).

As shown by comparing the intervention instructions 1383 for pneumothorax with the intervention instructions 1393 for cardiac tamponade, the next best steps (e.g., the steps following step 2 and proceeding to step N in FIG. 12) are different and in some cases opposite depending on the cardiac tamponade or pneumothorax determination. For example, for a pneumothorax, fluid delivery 1387 would be recommended to the caregiver whereas for cardiac tamponade, fluid delivery would not be recommended to the caregiver (e.g., no fluid delivery 1397). As an additional example, for pneumothorax not providing ventilation 1389 would be recommended to the caregiver whereas for cardiac tamponade ventilation 1399 recommended to the caregiver. Finally, for an indication of pneumothorax, a needle decompression 1385 would be recommended to the caregiver whereas for cardiac tamponade, an administration of vasopressors 1395 would be recommended to the caregiver. The CSG engine 150 may provide guidance for any or all of these procedures through the CSG UI 155 exemplified and discussed in regard to FIGS. 14B-1, 14B-2, and 14B-3.

As demonstrated by the example in FIG. 13, a single type of traumatic injury, chest trauma, may result in different presenting condition for the trauma victim with different and incompatible interventions. Thus, a system that can accurately and quickly analyze a substantial amount of data from the patient and the emergency care environment is substantially more likely to provide the correct intervention outcome. These chest trauma conditions can result in the death of a trauma victim within minutes thus the critical need for speed in accurately analyzing indicators and guiding care.

Referring to FIG. 14A-1, an example of CSG user interface is shown. A quantity of each component in FIG. 14A-1 is an example only and other quantities of each, or any, component could be used.

The mobile device 110 may provide a secondary display for one or more medical devices and provide patient treatment information associated with multiple medical treatment devices and/or other data, such as caregiver performance data and patient physiologic data along with CSG. Whereas each medical device provides data specific to that device or collected by that device, the data at each medical device provides a subsystem view and not a holistic view of the patient. In contrast, by aggregating the data from multiple medical devices at the mobile device 110 and providing centralized analysis by the CSG engine 150, the CSG system described herein provides treatment and/or guidance for a user to treat the patient as a whole and does not merely respond to a single subsystem, like a particular medical device. Furthermore, the physical location of the mobile device 110 may not be constrained by a need to couple to the patient as is the case with the medical devices. The medical devices generally require a particular and proximate position relative to the patient in order that sensors and therapy delivery devices may be properly coupled to the patient.

In some examples, the caregiver 103 can view patient treatment information received from the medical equipment 170 communicatively coupled to the mobile device 110. In addition, in some embodiments, the user interface views at the mobile device 110 may include user inputs that allow the caregiver 103 to transmit instruction signals to provide data or issue commands to control one or more functional operations of the medical equipment 170.

In some embodiments, providing the ability for a single caregiver to view case information from multiple medical treatment devices provides a technical solution to a clinical problem in that a caregiver and/or supervisor using the mobile device 110 can provide enhanced coaching and support to other rescuers. Further, in certain emergency care situations where patient access or rescue personnel are limited, providing a single caregiver with the ability to view case information simultaneously from multiple medical treatment devices and transmit instruction signals to control operation of the medical treatment devices enables patients to receive advanced medical care in remote or non-traditional care environments. However, some embodiments include various roles for various users. In some embodiments, a local wireless communication channel can be established among two or more of the devices in the emergency environment 120 to enable data to be securely and accurately shared among the devices and systems. For instance, health data about the patient 101, data indicative of treatment delivered to the patient 101, or other types of data can be exchanged over a wireless communication channel, potentially including one or more remote and offsite computing systems. This can help enable treatment by multiple rescuers to be coordinated or integrated in an efficient and accurate manner. In some examples, wired and/or wireless communication channels are established among only some of the devices involved in treatment of the patient 101 (e.g., between two of the devices). In some examples, a wireless communication channel is established among all of the devices involved in treatment of the patient 101.

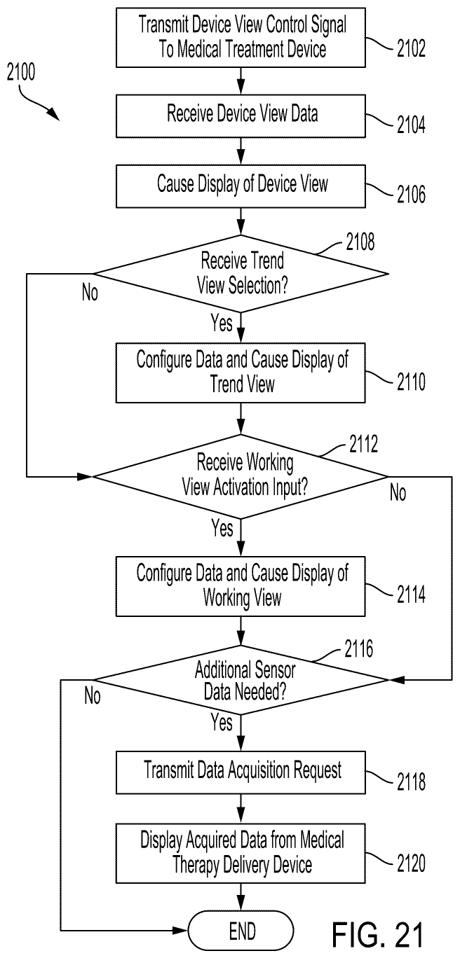

In an implementation, the mobile device 110 may provide a UI with selection tabs for multiple window views. For example, the selection tabs may include a device view selection tab 1410, a working view selection tab 1412, a trends view selection tab 1414, and a context-sensitive guidance selection tab 1416. The method 2100, as discussed below in regard to FIG. 21, is an example of a method that enables a display of the device view window, the working view window, and the trend view window.

The device view selection tab 1410 enables the mobile device user to access a device view window configured to provide a real time display of at least a portion of the plurality of physiologic parameters in a visual reproduction of a display format provided on a respective medical device at the operational interface of that respective medical device. A medical device may include an integrated display screen configured to display medical device data for the care of the patient. This integrated display screen provides an operational interface for the medical device. It allows the user to control the medical device operations and to view data collected by the medical device from the patient in real time as the data is collected. The device view window provides all of the data from the operational interface at a second display (i.e., the mobile device display 106) that is physically separate or separable from the medical device. The presentation of data at the device view window may replicate the presentation on the medical device or may rearrange or otherwise alter the appearance of the presented data.

The device view may improve care from a team of caregivers where the screen of the medical device may not be readily accessible to all team members and/or may be inconveniently located for optimum viewing (e.g., under a gurney, on a moving gurney, under an ambulance bench, etc.). For example, a first caregiver may attend to the use of the medical device to provide therapy to the victim. A second caregiver 104 may prepare medications, or a gurney, or attend to another task out of view of the medical device display screen. In some scenarios, the second caregiver may supervise a larger care team and possibly other victims. This second caregiver may provide care of the victim based on the device window view at the mobile device 110. Thus, the device view, allows additional medical team members to participate in patient treatment without having to be immediately proximate to the medical treatment device because they can view all the patient data concurrently displayed at the medical treatment device. In an implementation, the device view may provide information from the operational interfaces of multiple medical devices.

The working view selection tab 1412 may provide a presentation of the data available on the medical device display in a manner that differs from that of the operational interface on the medical device display. As an example, the working view window accessed via the working view selection tab 1412 may provide a different collection of data, a different arrangement of data, instructions and options not available on the device display, etc. and their view may be customized to the user of the mobile device and/or customized based on the status of the patient. In an implementation, the tab 1412 may be referred to based on a host device nomenclature. As examples, if the mobile device is a tablet, this tab may be labeled a “tablet view,” if the mobile device is a smartphone, this tab may be labeled a “phone view,” if the mobile device is a headset, this tab may be labeled a “headset view,” etc.

The working view window may display different data than the device view window. Rescuers and other medical team members may also wish to view additional information or information in a different format from what is provided in the device view window. The working view window may provide one or more patient data dashboards customized to preferences or treatment roles of a user of the mobile device. For example, a CPR data dashboard may display real-time CPR information, for example, including chest compression depth and chest compression rate. The displayed CPR information may provide real-time feedback to a rescuer delivering CPR chest compressions to a patient regarding whether the depth of each chest compression is within a target range and a target compression rate. Similarly, in some embodiments, a ventilation dashboard may display real-time ventilation information including, in some examples, ventilation rate, tidal volume, and minute volume. In some situations, a rescuer providing ventilation to a patient may select to have the ventilation dashboard displayed within the working view to more easily view feedback associated with providing patient ventilation. In some cases, the working view may be created and customized ad hoc during medical treatment by controls that allow the viewer of the mobile device 110 to format the medical device data as they wish.

In some examples, the working view window may be a scrollable display window that includes data dashboards displaying treatment-based sensor data associated with treatment devices or methods instead of or in addition to the information displayed within the device view. The scrolling capability increases the display real-estate available for data. In an effort to reduce the size and weight of a medical device to enhance portability and usability, the size(s) of the information display screen(s) on a medical device is often reduced to the point where the display area is too small to concurrently display all medical device data, including the sensor information gathered from the patient as well as all the information gathered about diagnostics, treatment, device performance, caregiver performance, etc. For example, while a patient monitor or defibrillator may only be a volume of less than approximately 0.5 ft.³ (approximately 0.01 m³) with a display size of approximately 8.5″ (approximately 22 cm) diagonal, the amount of information gathered and generated by the medical device at any one time could easily fill an entire display screen of approximately 36″ (approximately 91 cm) diagonal or more. This presents a dilemma for the caregiver using the medical device: the choice of what information to display is oftentimes difficult and displaying one particular parameter comes at the expense of losing visibility of another parameter. In addition, information that is not visible at a particular time during patient care may result in ineffective or harmful interventions and medical treatments for the patient. Thus, the working view window provides a page free of the medical device display area limitations.

The trend view selection tab 1414 enables the user of the mobile device 110 to access a trend view window. In an implementation, the trend view window is configured to provide user-customizable data trend information for one or more physiologic parameters received at the mobile device from one or more communicatively coupled medical devices. For example, upon selecting the trends tab 1414, the mobile device 110 may display a list of one or more trends for the user to select and/or de-select based upon trend viewing preferences. The trends view window may display physiological sensor value trends over time during the medical event. In some cases, the trend data can be displayed in a graphical or tabular format. The trends view window may be a scrolling window so that users can access a larger set of data with a scrolling gesture. The trend view window 1515 may also include discrete data values. In one example, user can select for display waveforms and/or mean values for physiologic parameters captured by the medical devices communicatively coupled to the mobile device. In some embodiments, the user can also select a time interval for recording trend data that is displayed within the trend view. In some implementations, inputs provided at the trend selection tab 1554 can also allow to select the predetermined time interval e.g., every 10 seconds, 20 seconds, 30 seconds, 1 minute, 3 minutes, 5 minutes) for recording and/or displaying trend data in the trends view window. In an implementation, the trends view window may include event marker annotations overlaid on the trend plots. These annotations enable the user to readily discern an impact of a respective intervention or treatment on a patient's condition over the course of time. In an implementation, the one or more medical equipment 170 may automatically record event markers for various treatments, interventions, or other activities during patient care provided to the patient or caregivers may manually input event marker data at the medical equipment 170 and/or at the mobile device 110. In an implementation, the CSG engine 150 may generate event markers. Event markers may indicate activities such as, for example, administration of a particular drug, placement of an IV, oxygen administration, detection of a shockable rhythm, application of electric shock to the patient, initiation of chest compressions, initiation of ventilations, etc.

In some situations, multiple caregivers may provide care for the victim. For example, after viewing one or more of the windows at the mobile device 110, a caregiver on scene but not directly administering care to the patient and/or the remote caregiver 389 may deem a particular piece of medical device data to be relevant to the decision by the caregiver directly administering care to the victim. In some scenarios, the caregiver may send an instruction from the mobile device 110 to the medical equipment 170 to effectuate a change to its operation and/or a change to the data display at the medical equipment 170. For example, the instruction may include a command to initiate a blood pressure reading via an oscillometric blood pressure cuff, if the supervisor notices that the time duration since the last blood pressure reading has been exceeded. As another example, a 12-lead may be initiated from the mobile device 110. As an example, while treating a trauma victim not initially presenting with respiratory distress, the caregiver attending to the trauma victim may have made the choice to not include either the capnography waveform or EtCO2 values on a display screen of the medical equipment 170. Another caregiver may see in the working view that the capnography waveform is indicative of an obstructive lung condition and toggles over to the device view and sees that the capnography information is not being displayed (upon which the first caregiver is basing their potentially flawed medical decisions). Based on this, the additional caregiver may initiate an instruction from a second mobile device to the mobile device 110 and/or to the medical equipment 170 to alter the display format and display the capnography information. Alternatively, the instruction may take the form of a request to alter the display format. The request may be in the form of a text request to the caregiver operating the medical treatment device (e.g., “Important patient info: please display capnograph”).

The CSG selection tab 1416 enables the user of the mobile device 110 to access a CSG UI. An example of a CSG UI 155 provided at the mobile device is shown in FIG. 14A-1. The design or layout of the CSG UI 155 and the constituent screens as illustrated herein are examples only and not limiting of the disclosure. The CSG UI 155 and the constituent screens include various interaction elements (e.g., entry fields, selectable menus, buttons, arrows, checkboxes, soft-keys, sliders, etc.) and associated displayed instructions to prompt the caregiver for a user entry of contextual data. The mobile device 110 may provide the user-entered contextual data as the input 980 to the CSG engine 150. Additionally, the CSG UI 155 and the constituent screens provide information included in the output 990 to caregiver interface devices. The output 990 includes information from the CSG engine 150 provided to the mobile device 110 for display at the CSG UI 155. Additionally, the CSG UI 155 may be a touch-activated interface controlled by the user via one of more touchscreen gestures 1698 and may include a scrolling window as described in regard to other windows at the mobile device 110.

In an implementation, interactions between a caregiver and the CSG UI 155 may include a voice command or utterance from the caregiver (e.g., the utterance to the voice control 1441 in FIG. 14A-1) and/or audible output (e.g., the output 1442 in FIG. 14A-1) from the CSG UI 155. In lieu of or in addition to the menu or keyboard entries illustrated herein for user entry of information, the CSG UI 155 may capture voice information and convert this information to text entries. In some implementations, the voice information may include annotations for event marker data. The CSG UI 155 may provide a digital assistant, as indicated for example by the icon or voice activated control 1440, to allow user control of and input to the CSG engine 150 to manipulate the CSG UI 155 via natural language processing. The voice command or utterance may conform to a pre-determined command list and/or may be an unstructured utterance that is interpreted by the natural language processor. For example, the caregiver may say “continue” or “next” in accordance with a pre-determined command list. Additionally, or alternatively, the caregiver may say “I need more help” or “what do I do now” as examples of unstructured text that the natural language processor converts to structured text of “continue” or “next.”

In an implementation, the caregiver 104 may activate the digital assistant with an utterance (e.g., the user may utter “Digital Assistant” or another command or moniker to activate voice control 1441). In response, the CSG engine 150 may provide the audible information or response 1442. For example, the CSG engine 150 may audibly query the user for more information or clarification (e.g., audible information may include a question such as “what would you like to do?”) and/or may audibly provide any or all of the instructions shown on the CSG UI 155 and/or one or more of the parameters available at the mobile device 110. The voice activated control 1441 may also provide the functions of one or more of the touchscreen controls for the CSG UI 155. For example, one or more of the audible utterances of “continue,” “cancel,” “exit,” “confirm,” “back,” “next,” “more guidance,”, “silence,” “connect device,” “back,” “working view,” “trends,” “device view,” or synonyms or other equivalent function terms and/or combinations thereof may cause the CSG engine 150 to perform the functions also associated with touch screen controls. For example, a “continue” touchscreen control enables an option to continue CSG instructions, a “next” touchscreen control enables an option to proceed to an next step in a series of CSG instructions for a clinical intervention, an “exit” touchscreen control enables an option to exit CSG instructions, a “more guidance” touchscreen control increase a detail level for CSG, a “back” touchscreen control enables a user to return to a previous instruction in a series of CSG instructions for a clinical intervention, and a “silence mode on/off” touchscreen control enables the user to mute or unmute audible UI output. The silence mode on/off control may be of critical importance in a military setting where noise could endanger a mission or provoke an assault and may be of critical importance in a noisy emergency environment where background noise may interfere with voice control or understanding of audible CSG instructions.

The scanner control 1452 may enable the mobile device 110 to capture information for the CSG UI 155 via a barcode scan, a QR code scan and/or a biometric scan. In one example, the user may activate the scanner control 1452 to scan a barcode of a medication being administered to the patient, and the mobile device 110 may convert the scanned identification code into medication type and/or dosage. In other examples, the user may activate the scanner control 1452 to scan demographic information, payment information, medical device identification codes, caregiver identification codes, patient and/or caregiver biometrics, etc. In an implementation, the user may verbally activate the scanner control 1452.

In an implementation, the CSG engine 150 may generate medical device operation instructions. The medical device operation instructions may be one or more of instructions for the caregiver or remote controls that enable automated instructions to be sent to the medical equipment 170 from the mobile device 110. In an implementation, the mobile device 110 may include one or more remote medical device controls 1481 at the UI 155 associated with the operation of a communicatively coupled medical device. In an implementation, the mobile device 110 can transmit instruction or command signals to the medical devices in response to a user activation of the medical device control 1481. For example, the CSG engine 150 may provide settings recommendation and the caregiver may implement those recommendations through an input to the mobile device 110. In an implementation, the mobile device 110 may transmit instruction or command signals to the medical devices automatically in response to a determination by the CSG engine 150 of particular medical device settings. The transmitted instruction signals from the mobile device 110 to the medical equipment 170 may provide information, initiate one or more functional operations, or set or change operational parameters or modes of the respective medical treatment device. In a further implementation, the caregiver may utilize controls physically located at the medical device(s) and the CSG UI 155 may display those settings 1482.

Referring to FIGS. 14B-1, 14B-2, and 14B-3 an example of a method for providing CSG at a user interface is shown. The method 1400 is, however, an example only and not limiting. The method 1400 can be altered, e.g., by having stages added, removed, rearranged, combined, and/or performed concurrently.

Starting with FIG. 14B-1, the mobile device 110 may display 8 one of the working view, trend view, or device view windows. The CSG engine 150 may detect 10 indicators of trauma in the contextual data. For example, the MOI, user input to the mobile device and/or medical devices, vital signs, the type of medical equipment in use, medical supplies selected for use by the caregivers, etc. may provide indicators of trauma. In response to the detected indicators of trauma in the contextual data, the CSG engine 150 may provide 12 a user notification at the mobile device display. The caregiver may select 12 the CSG window selection tab 1416. In response to this selection, the CSG engine 150 may modify the display at the mobile device 110 to provide the CSG UI 155. In an implementation, the CSG engine may provide the CSG UI 155 automatically in response to a detection of a likely trauma in the contextual data and then giver the user an option to exit from this display.

Unlike the information provided at the display screen of a typical medical device or patient monitor, the CSG engine 150 may curate the information provided to the caregiver. The large amount of data that may be provided at a medical device display screen, such as a patient monitor screen, shows an overall state of a patient. These data displays may not provide caregiver guidance. Rather, the caregiver has to sort, analyze, and evaluate this large amount of data to determine if clinical interventions are necessary. As is commonly the case for emergency medical services, particularly in the pre-hospital context, the caregiver may have multiple patients and other distractions (e.g., family members, moving the patient, care activities, environmental distractions, etc.). Thus, in contrast to the medical device screen information, the CSG engine 150 does not merely provide information for the caregiver to view, analyze, and draw conclusions from. Rather, the CSG engine 150 selects particular information, i.e., critical physiologic parameters, that the caregiver needs to or should be aware of at a particular moment and selects and instructs the caregiver on immediate interventions. The CSG engine 150 may downregulate or filter the large quantity of data available from a typical medical device to specific factors and steps that are immediately necessary to increase the odds of patient survival. This real-time context sensitive guidance from the CSG engine 150 provides the caregiver with an understanding of the purpose and urgency of those interventions. The CSG engine 150 curates the information provided to the caregiver at the CSG UI 155. For example, the CSG UI 155 may exclude physiologic parameters other than the critical physiologic parameters to emphasize the critical physiologic parameters. For example, a heart rate between 100-120 bpm and a respiratory rate between 20-24 bpm may indicate hemorrhage. The CSG UI 155 may display these two physiologic parameters and exclude other parameters to focus the caregiver's attention on this issue along with a recommendation to address the source or cause of hemorrhage. In an implementation, as a condition changes, the CSG engine 150 may add additional critical parameters to the display at the UI 155. For example, as hemorrhage worsens, there may be a drop in blood pressure (e.g., a drop below 90 mmHg) and the CSG engine 150 may add the blood pressure measurement to the display of critical parameters. However, the selection tabs 1410, 1412, and 1414 allow the user to toggle to any other window and view all of the physiologic parameters available at the mobile device 110. Additionally, the exit control 1461 enables the user to exit the CSG UI 155 and return to a previously engaged window view.

In various implementation, the CSG UI 155 may utilize graphical tools to guide the caregiver. For example, for a series of activities, the CSG UI 155 may provide a progress bar with a pointer to remind the caregiver of a position of a current clinical intervention within the series. In some examples, the CSG UI 155 is configured to a visually distinguish between a current step in an instruction list and one or more of subsequent and previous steps in the instruction list with an expanded detail window, differing bullet icons, and various combinations of colors, fonts, grey scale, bold type, character size, etc. to distinguish between a current step and upcoming steps. For example, as each instruction becomes a current instruction, the CSG UI 155 may provide the current instruction in bold face and provide the other instructions in a lighter gray scale face. By visually distinguishing between the steps, the CSG UI 155 may focus the caregiver's attention on a current step while still providing information about an entire sequence of steps. Further, additional details associated with a current step may be visual distinct from subsequent steps and may match the visual appearance of the current step. As yet another example, the steps may be distinguished from one another by physical placement and by numerical indicators.

As another example of presentation of CSG details, the CSG UI 155 may provide these as graphic images. The graphic images may be an illustrated depiction of the medical equipment or a photographic image of the medical equipment. In an implementation, the graphic images may include animated instructions or videos. The graphic images, either illustrated or photographic, may include instructional overlays, such as arrows, words, and pointers etc. that indicate a method of use and/or assembly for the medical equipment. In an implementation, the CSG engine 150 may access stored graphic images of medical equipment based on the connected devices information. For example, the CSG engine 150 may tailor or match the images to a particular make and model of medical device as indicated by the connected devices information. Additionally, the CSG UI 155 may provide the graphic images and/or the instructional overlays in color, either in whole or in part. Further, the instructions corresponding to color images may be rendered in colors that match the images and/or the instructional overlays. The graphic images (e.g., illustrated depictions, photographic images, or a combination thereof) may include and/or depict instructions for caregiver use of the medical device. The CSG UI 155 may include alphanumeric instructions and/or identification information with the graphic images. The graphic images may include expanded view windows that are associated with the listed instructions and that change in association with and according to changes in emphasis on the listed instructions. The graphic images also include varying amounts of detail according to the detail provided in listed instructions. As the CSG UI 155 changes the emphasized portion of the clinical intervention instructions to guide a caregiver, the CSG UI 155 also changes the equipment images and expanded views to illustrate the instructions. As another example, the graphic images may include images of a patient where necessary to illustrate proper attachment of a medical sensor or device to the patient.

As an example, the CSG engine 150 may modify one or more of the device view, the working view, the trend view, or the CSG view to include a user notification 1425 of a likely trauma condition. For example, the MOI may indicate that trauma is likely. In an implementation, a physiologic parameter measurement may provide an indication to the system that a traumatic injury is likely. For example, a sharply pointed capnography waveform (e.g., often referred to as a “shark-fin” waveform), a precipitous drop in blood pressure, tachycardia, etc. In an implementation, the CSG engine 150 may provide the user notification 1425 at the window currently viewed by the user of the mobile device (e.g., the device view, the trend view, the working view, or the CSG view). In an implementation, the CSG engine 150 may control the display screen 106 of the mobile device 110 to provide the user notification window 1425 in an unoccupied portion of the currently displayed window to emphasize and highlight the detected change and the critical physiologic parameters. In this way, the notification grabs the attention of the user without obscuring any other available information.

In an implementation, the CSG UI 155 may provide the critical physiologic parameters along with clinical intervention instructions. In an implementation, the CSG UI 155 may provide only the critical physiologic parameters and exclude other physiologic parameters in each screen view that includes clinical intervention instructions in order to keep the caregiver focused on these parameters without the distraction of other physiologic information and to minimize information interpretation tasks for the caregiver. In an implementation, the CSG UI 155 may provide the critical physiologic parameters in a consistent and same location on the window view in order to minimize confusion and information interpretation tasks for the caregiver.

With the user notification 1425, the CSG engine 150 may provide one or more controls for guidance selection options. For example, the CSG engine 150 may provide the continue control 1450 for an option to continue instructions and/or the cancel control 1451 for an option skip guidance. In an implementation, activation of the continue control 1450 automatically transitions the display at the mobile device 110 to the CSG UI 155. In an implementation, activation of the cancel control 1451 may clear the user notification 1425 and/or the display of critical parameters.

Returning again to FIG. 14B-1, the CSG engine 150 can control the CSG UI 155 to provide a sequence of UI screen displays in a manner customized to the needs of the caregiver. As an example, the CSG UI 155 may provide 18 a trauma intervention overview for that includes one or more stages (e.g., stage 1, stage 2, . . . , stage N). The overview may apply to a generalized trauma intervention or to a specific traumatic injury. The overview may list a sequence of one or more steps that the caregiver should take to implement the trauma intervention. In various implementation, the overview may conform to the preferences and practices of a particular medical director and agency and/or may provide guidance according to a standard of care that may be common across multiple caregiver organizations and may or may not provide the same detail with regard to components like drug dosages or an order of operation. Other changes to the overview and associated steps may include one or more of adding more steps, adding more caregiver questions, changing steps, changing the order of the steps, selecting different critical physiologic parameters or data as a trigger for CSG, selecting different data as the critical data, etc.

For example, a trauma intervention overview for a pneumothorax may include the steps of (1) conducting a stethoscope or ultrasound exam, (2) provide a needle decompression and (3) provide fluids. As another example, a trauma intervention overview for a cardiac tamponade may include the steps of (1) conducting a stethoscope or ultrasound exam, (2) provide a vasopressor, and (3) ventilate.

The CSG UI 155 may request a confirmation 24 that the caregiver has seen the overview. For example, the CSG UI 155 may provide a confirmation control 1455. At the overview stage, the caregiver may select 28 to continue with CSG or exit. For example, the exit control 1454 may return the mobile device to a display of the working view, trend view, or device view window. If the caregiver. If the caregiver selects to continue with CSG, the CSG UI 155 may provide 30 intervention instructions for stage 1 of the overview. Similarly, the CSG UI 155 may provide intervention instructions for stage 2 through stage N, e.g., at the stages 38 and 46. Following each set of instructions, the CSG UI 155 may request a confirmation, e.g., the confirmation 40 after the instructions for stage 2 and the confirmation 48 after the instructions for stage N. The confirmation enables the caregiver to confirm that an instructed intervention has been provided to the patient by the caregiver or by a medical device. In an implementation, activation of a confirmation generates a code marker in a case file for the patient encounter. The mobile device 110 may provide an indication of the confirmation to at least one of the medical devices along with an event code indicative of the activity or intervention that is confirmed, and the medical device may record the code marker in the case file. In an implementation, in order to advance through the CSG screens, the CSG engine 150 may require a confirmation that a current step has been completed before moving to the next step.

In an implementation, the CSG engine 150 may prompt the caregiver for a confirmation that a previously instructed action item or next best step prior to an evaluation of the efficacy of that prompted action item of next best step. For example, if an action item should improve a patient care state and the system cannot determined based on the inputs 980 and 985 whether this action item has occurred, it may request the confirmation. This may avoid an erroneous determination thus an action item was ineffective and should be modified or repeated due to the lack of state change. In an implementation, the CSG engine 150 may flag detrimental changes in the physiologic condition of the victim based on the physiologic data and/or the environmental data. Further, the CSG engine 150 may provide instructions to the caregiver and/or the medical devices in direct response to this detrimental change in order to provide correction. In some instances, such a detrimental change may indicate improper performance of an action item rather than ineffectiveness or non-performance. In providing this correction, the CSG engine 150 may identify a previously performed action item that is or is likely to be associated with the detrimental change. The CSG engine 150 may modify any previously determined order of performance for the next best steps to return to this previously performed step and either repeat or modify the action item. For example, a modification may include a change in dosage of a medication.

Moving to FIG. 14B-2, at each stage, the CSG UI 155 may enable the caregiver to select to progress to a next stage (e.g., via the next control 1460), exit CSG (e.g., via the exit control 1454), or receive additional guidance (e.g., via the additional guidance control 1456), e.g., at the stages 34 and 42. At the last stage N, the CSG UI 155 may enable the caregiver to receive additional guidance or exit CSG, e.g., at the stage 50. The additional guidance may be more granular instructions. A caregiver of higher skill or longer experience may need less granular information than a lower skilled or less experienced caregiver. If the caregiver selects additional guidance, then the CSG engine 150 provides a sequence of one or more screens that provide a breakdown of activities associated with an intervention instruction.

Continuing with FIG. 14B-2 and moving to FIG. 14B-3, upon a selection of additional guidance, e.g., using the additional guidance control 1456, the CSG engine 150 may provide a more detailed set of instructions than the top-level instructions included in the overview. The CSG engine 150 may provide a nested set of additional detail instructions A, B, . . . , M (e.g., as shown at the steps 64, 74, and 84 in the method 1400). For simplicity, FIGS. 14B-2 and 14B-3 shows instructions labeled as “A,” “B,” and “M” as available for stage 1, stage 2, and stage N. However, the instructions “A,” “B,” and “M” associated with each stage are not the same. Rather, these instructions provide a granular breakdown for their associated stage. Additionally, one or more of the steps A, B, . . . , M may also be associated with further nested set of more granular instructions for each of these steps.

For example, the additional guidance for an overview step of provide a needle decompression may include the steps of prepare site, insert needle, remove needle, and secure catheter. In contrast, the additional guidance for an overview step of provide a vasopressor may include the steps of insert venous catheter, couple infusion pump to catheter, initiate medication delivery, remove catheter.

The granular instructions “A,” “B,” and “M” may include instructions for caregiver activities that may include use and assembly of a medical device (e.g., a tourniquet, a ventilator, a stethoscope, an ultrasound device, an intravenous fluid delivery device, a needle catheter, a drug delivery device, an infusion pump, a venous catheter, etc.), administration of medication, steps for monitoring a patient, steps for a particular diagnostic tool (e.g., ultrasound), etc. The instructions for use may include basic instructions for less skilled caregivers or caregivers with limited experience with a particular type of medical device or a particular make and model of a medical device (e.g., a power-on instruction or a mode selection instruction). The CSG engine 150 also enables the caregiver to control a pace and timing of provided instructions. The caregiver may need to control the pace and timing because of their skill level and/or because of other emergency activities that may emerge during the guidance.

After each set of additional instructions, the CSG engine 150 may request confirmation, e.g., at the stages 66, 76, and 86. Further after each instance of additional instructions (other than the first and last instances), at the stage 76, the CSG engine 150 may enable the caregiver to progress to a subsequent instruction, e.g., via the next control 1460 or go back to a previous instance, e.g., via the back control 1461, return to the stage in progress, e.g., via the return control 1462 or exit CSG. The return control may return instructions to the instruction stage from which the additional guidance request originated (e.g., one of stage 30, 38, or 46). In an implementation, the CSG UI 155 may return to the instruction stage from which the additional guidance request originated by default in the absence of another selection by the caregiver. The exit control takes the UI back to the device view, the working view, or the trend view. At the first instance and the stage 68, the CSG engine 150 may enable the caregiver to go to a next instruction, return, or exit. At the last instance and the stage 88, the CSG engine 150 may enable the caregiver to go back to a previous instance, return, or exit.

Any or all of the stages 1, 2, . . . , N and/or the additional guidance instructions A, B, . . . , M, may include drug administration instructions. In conjunction with an instruction to administer a pharmacologic intervention, the CSG UI 155 may provide dosing instructions and/or reminders 1470, drug delivery confirmation controls 1473, and/or a dose interval timer 1475. In an implementation, the dosage timer 1475 may be a graphic feature that fills gradually or changes color gradually to indicate an elapsed time between medication dosages. For example, the fraction of an area of the graphic feature 1475 that is filled and/or changed color compared to the total area of the UI feature may indicate an amount of time elapsed or remaining. The CSG engine 150 may automatically determine the length of the interval between medication doses. For example, the caregiver may scan a bar code on a medication using a camera or scanner coupled to the CSG engine 150. The CSG engine 150 may identify the medication based on the bar code scan and may combine that information with demographic patient information (e.g., age, weight, gender, etc.) to determine a medication dosage and dosage timing. The CSG engine 150 may automatically set the timer according to the determined dosage and timing. In an implementation, the CSG engine 150 may provide a workflow guidance window 1435, for example the banner as shown in FIG. 14A-2. The workflow guidance window 1435 may provide specific workflow instructions and may track a confirmation with the confirmation control 1436. In an example, the workflow guidance window 1435 may provide drug delivery instructions and dose counting in lieu of the counters and timers 1473 and 1475. In an example, one or more of the counters and times 1473 and 1475 may be included in the workflow guidance window 1435.

In an implementation, the CSG engine 150 may provide an incomplete treatment explanation window 1437, for example the pop-up window as shown in FIG. 14A-2. For example, if a caregiver omits or does not provide a recommended treatment or intervention, the CSG engine 150 may remind the caregiver that the treatment or intervention is incomplete and collect information relevant to the incomplete treatment or intervention. The incomplete treatment explanation window 1437 may enable a caregiver to restart a treatment or intervention or may enable a caregiver to indicate one or more reasons why the patient cannot receive the particular treatment or intervention. These one or more reasons become part of the contextual data used by the CSG engine 150 to determine a next best step (e.g., at the stages 1205, 1207, 1209, and/or 1305 as shown for example in FIGS. 12 and 13).

In an implementation, the CSG UI 155 may include a silence mode on/off control 1457. The silence mode on/off control 1457 enables the user to mute or unmute audible UI output. The silence mode on/off control 1457 may be of critical importance in a military setting where noise could endanger a mission or provoke an assault and may be of critical importance in a noisy emergency environment where background noise may interfere with voice control or understanding of audible CSG instructions.

Referring to FIG. 14C with further reference to FIGS. 14D-1, an example of a UI population process for a CSG UI, for example a CSG UI, and examples of UI features are shown. At the stage 2410, the CSG engine 150 populates the CSG UI 155 with available devices on the ambulance (e.g., the window 3410 in FIG. 14D-1).

At the stage 2415, the CSG engine 150 receives information from one or more of the emergency dispatch services 130 (e.g., the emergency event notification information 135) and from the caregiver (e.g., via input to the CSG UI 155) regarding the victim and the emergency medical incident. The victim information may include demographics and/or known or observed patient symptoms. The trauma incident information may include a mechanism of injury (MOI) and granular details surrounding the MOI. In an implementation, the CSG engine 150 may receive patient conditions at the stage 2420. For example, the UI 155 may provide a menu of conditions 3420. The menu 3420 may enable selection by the caregiver and/or may automatically indicate information received by the CSG engine 150 from the emergency dispatch service 130. For example, the menu may highlight one or more conditions automatically and/or may highlight one or more conditions as the caregiver activates the menu control. For example, as shown in FIG. 14D-1, the CSG engine 150 may automatically highlight the “trauma” control and the caregiver may use a touchscreen gesture or a verbal command to highlight the “pediatric” control. In an implementation, the CSG UI 155 may not display the menu 3420 and may populate reminders and protocols automatically without use of a displayed menu 3420.

Similarly, and as shown in FIG. 14D-2, the CSG UI 155 may display and/or collect demographic and mechanism of injury (MOI) information at a control windows 3425 and 3430. For example, the trauma victim may be a 57-year-old male with blunt injuries due to a motor vehicle collision (MVC).

As shown in FIG. 14D-3, the CSG engine 150 may collect, store, and/or display at the UI 155 granular information 3435 about the MOI, for example MVC details. This granular information may determine the priorities 3412, protocols 3414, and reminders 3416. For example, the traumatic injuries sustained by a victim of a low-speed collision as a bus passenger with a seat belt restraint may be dramatically different that the traumatic injuries sustained by a victim of a highway speed collision as a motorcycle driver thrown from the motorcycle.

At the stage 2420, the CSG engine 150 receives patient symptom and state information. For example, as shown in FIG. 14D-4, the CSG engine 150 may collect, store, and/or display at the UI 155 patient symptoms and states 3440. The CSG engine 150 may receive the patient symptom and state information via one or more of voice input, touchscreen input, medical device input, charting input, and medical history input.

At the stage 2430, the protocol loading engine 515 may load one or more protocols. For example, as shown in FIG. 14D-4, the protocols may include a rapid trauma survey protocol 915, a spinal motion restriction protocol 930, and an International Trauma Life Support (ITLS) initial assessment protocol 906. In an implementation, the protocol window 3414 may provide caregiver access to all or a portion of the loaded protocols as a reference tool.

The patient priorities 3412 may appear as a list of prioritized interventions. The protocols 3414 and reminders 3416 features may be controls that cause the UI to display protocol and reminder information upon activation of the control by the caregiver, either verbally or through tactile interaction. The CSG engine 150 determines the patient priorities 3412, protocols 3414, and reminders 3416 based on the information received at the stages 2415 and 2420.

At the stage 2435, the CSG UI 155 may prompt the user to request guidance (e.g., via the controls 3445 shown in FIG. 14D-5. At the stage 2440, the CSG UI 155 may provide prompts for additional information 3450. At the stage 2445, the CSG UI 155 may provide one or more patient priorities 3412. Additionally, the CSG UI 155 may highlight the medical devices needed to implement interventions or monitor conditions relevant to the patient priorities (e.g., the highlights 3455A and 3455B shown in FIG. 14D-5). The CSG UI 155 may provide prompts verbally and/or visually and may receive caregiver input verbally and/or through touchscreen gestures. In implementation, the prompts for additional information may include caregiver confirmation of information received from dispatch.

At the stage 2450, the CSG engine 150 recognizes that a medical device is activated (i.e., attached to a patient and actively monitoring and/or providing therapy). At the stage 2455, the CSG engine 150 automatically receives the medical device input. As shown in FIG. 14D-6, once an item of medical equipment is attached to a patient and actively monitoring and/or providing therapy, the image of the equipment may be replaced by an image 3460a and 3460b of an operational interface of that equipment. The original equipment icons and/or the images 3460a and 3460b may be controls that provide an expanded caregiver view 3462 of the information currently displayed at the medical device upon activation of these controls. The expanded caregiver view 3462 may be a device view window within the CSG window configured to provide a real time view of the physiologic data as a visual reproduction of a display format for the at least one medical device communicatively coupled to the CSG engine. Providing the visual reproduction may include replicating the display format of the at least one medical device. Providing the visual reproduction may include rendering a display of the physiologic data in which one or more visual aspects of the physiologic data are different than the display format of the at least one medical device. The device view window may provide a real time view of the physiologic data provided at the at least one medical device. In an example, the device view window may include an ECG waveform, a pulse oximetry waveform, invasive blood pressure (IBP) and/or a CO2 waveform. The device view window may include current values for at least one of peripheral capillary oxygen saturation (SpO2), carbon monoxide saturation (SpCO), methemoglobin (SpMet), total hemoglobin (SpHB), blood oxygen content (SpOC), pleth variability index (PVI), perfusion index (PI), end-tidal carbon dioxide (ETCO2), non-invasive blood pressure, invasive blood pressure value, heart rate (HR), respiration rate, fraction of inspired oxygen (FiO2), and temperature . . .

In an implementation, as shown for example in FIG. 14D-7, the expanded caregiver view 3462 of the information from the medical device may include information from multiple medical devices. In the example of FIG. 14D-7, the expanded caregiver view 3462 includes information from the defibrillator device corresponding to the icon 3455a and from the ventilation device corresponding to the icon 3455c. The data within bracket 3486 corresponds to the defibrillation device and the data within bracket 3484 corresponds to the ventilation device. The CSG UI 155 may include device markers (e.g., the device markers 3480a and 3480b) that identify the source of medical device data shown on the CSG UI 155. Device data may be added to and/or removed from the CSG UI 155 as medical device statuses change from available and connected to unavailable and/or disconnected and vice versa. For example, if only the defibrillation device is connected to the CSG engine 150, the CSG UI 155 may only display the data in bracket 3486. When the ventilation device becomes communicatively coupled to the CSG engine 150, the CSG UI 155 may add the data within the bracket 3484. Furthermore, if the ventilation device becomes disconnected from the CSG engine 150, the CSG UI 155 may remove the display of data within the bracket 3484. Furthermore, the CSG UI 155 may provide a scroll control 3482 to enable a user to scroll through data from multiple devices rather than requiring the CSG UI 155 to reduce the size of data in order to fit all of the data on a single screen view.

The CSG UI 155 may also provide automatically determined assessments 3465. Additionally, the CSG engine 150 may track uncompleted steps and/or steps that can be performed at a later time as reminders 3416. The CSG engine 150 may display reminders and/or may provide them as prompts.

At the stage 2460, the CSG UI 155 may provide next best steps. For example, based on a patient not breathing and exhibiting hypoxia, the next best step may be to provide oxygen (e.g., the next best steps 3470 in FIG. 14D-8. The CSG engine 150 may continue to receive patient condition information at the stage 2420 and update and adjust protocols, medical devices, priorities, and next best steps as needed based on the changes and updates to the patient condition information. In an implementation, the CSG engine 150 may receive updates to the available medical devices, the victim demographics, the victim symptoms, and the MOI and thus may repeat one or more of the stages 2410 and 2415.

Referring to FIGS. 14E-1 and 14E-2, examples of alert windows for the CSG UI are shown. A quantity of each component in FIGS. 14E-1 and 14E-2 is an example only and other quantities of each, or any, component could be used. The CSG engine 150 may generate the alert windows based on one or more of a patient's monitored physiologic data, which may include vital signs, ECG, or other physiologic data specific to a particular intervention. The alert windows may be provided as banners in a prominent location on the CSG UI, such as at the top of the UI display.

As shown in FIG. 14E-1, in an example, an alert window 3418a may include an alert based on contextual data to provide a particular intervention for the patient (e.g., a high flow nasal cannula). However, based on monitored data for the patient, the CSG engine 150 may determine that the monitored data is or is not showing an expected result of the particular intervention. If the monitored data is not showing the expected result, then the CSG engine may re-evaluate the data and recommend one or more subsequent interventions with the alert windows 3418b and 3418c. In an example, contextual data may indicate the presence of external bleeding, or hemorrhage. In response, the alert window 3418a may provide an alert to apply a tourniquet. The CSG engine 150 may then monitor vital signs and provide one or more alerts to adjust the tourniquet (e.g., the window 3418b) or to re-adjust or replace the tourniquet (e.g., the window 3418c) in a case where the monitored patient vital signs are inconsistent with proper tourniquet application. These alerts may enable a caregiver to apply the tourniquet and then attend to other critical tasks while still maintaining effective patient care based on the alerts. Because the CSG engine 150 can monitor a larger volume of contextual data than a caregiver and can look for correlations and trends in that larger volume of data, the system 145 may provide an alert of an ineffective treatment before this becomes obvious to the caregiver. As such, the CSG engine 150 may function as an early warning system and enable the caregiver to adjust or alter an intervention before there is harm to the patient.

As shown in FIG. 14E-2, in an example, an alert window 3418d may include an alert based on contextual data to consider early or immediate patient transport. In some cases, a caregiver must decide how much medical treatment to provide on-scene and prior to loading the patient into a transport vehicle, such as an ambulance or helicopter, for transport to a medical facility. The contextual data may include physiologic data for the patient. In an implementation, the contextual data may further include one or more of distances to medical facilities, types of care available at those medical facilities, insurance or pre-approval information for the patient, preferred provider information for the patient based on insurance or other medical payment assistance plan, the conditions on scene (e.g., weather, likelihood of further injury, etc.), and so forth. The CSG engine 150 may evaluate this data to generate an alert that a next best step, where possible, is to transport the patient rather than treat the patient on scene.

As further shown in FIG. 14E-2, in an example, an alert window 3418e may indicate that a complete dosage of a particular medication has been delivered and/or that remaining doses are available. The CSG engine 150 may generate the alert 3418e based on the medication dosage counter 1473 and time 1475 as shown for example in FIG. 14A-1.

As shown in FIG. 14E-3, in an example, the CSG UI may provide a scrollable notification window, for example the notification windows 3419a, 3419b, and 3419c. The scrollable notification windows 3419a, 3419b, and 3419c may include alerts, workflow guidance, protocol guidance, reminders, and/or other caregiver notifications generated by the CSG engine 150. The scrollable notification window may include a notification counter 3417 that indicates the total number of notifications and the sequential order of an individual notification of the total number of notifications. Further, the scrollable notification window includes a scrolling control 3415 that enables a user of the UI to scroll through multiple notifications associated with an encounter or user session. This enables the CSG UI to display only a most recent notification to reduce user distraction while still enabling the user to access prior notifications. Additionally, as shown in the scrollable notification window 3419a, the notification counter 3417 may indicate that zero notifications have been provided. This enables a user to verify and confirm that the user has not missed any notifications.

FIG. 15 shows an example of an AI modeling engine of a CSG engine. A quantity of each component in FIG. 15 is an example only and other quantities of each, or any, component could be used. As shown, the AI modeling engine 520 of the CSG engine 150 includes a channel handler 1505, an audio processing engine 1510, a natural language processing engine 1520, and, optionally, a computer vision processing engine 1530.

In an implementation, the contextual data sources 899 may collect and provide raw unstructured data to the AI modeling engine 520. The channel handler 1505 may sort the unstructured data into AI sub-fields such as audio, text, or image data and provide the unstructured data to an appropriate AI processing sub-engine. Each AI processing sub-engine processes a single mode of unstructured data. For example, the audio processing engine 1510 processes the audio data, the natural language processing (NLP) engine 1520 processes the text data, and the computer vision (CV) processing engine 1530 processes the image data. The image data may include still and/or video images. Each AI sub-engine includes trained models that convert the unstructured input into structured probabilistic output. Probabilistic output refers to a confidence metric associated with the output. In other words, the AI engine 520 generates both an output and in indication of how confident the engine 520 is that the output is correct.

The output from the AI engine 520 is post-processed by the CSG engine 150, specifically by one or the audio post-processing engine 1540, the NLP post-processing engine 1550, and the CV post-processing engine 1560. The post-processing engines may utilize the structured output to provide the functions of the CSG engine 150. For example, the post-processing engines may include a clinical finding handler that recognizes the structured output as a clinical finding or diagnosis. Based on this clinical finding or diagnosis, the CSG engine 150 may predict a next best step and generate guidance in the form of an instruction to perform the next best step. The clinical finding handler may include sub-handlers for various subcategories of clinical findings. For example, the subcategories may be organized according to physiologic systems and/or protocol, such as airway, breathing, bleeding, etc.

The engines 1540, 1550, and 1560 may provide a response to the channel handler 1505 which in turn routes that response to the appropriate contextual data source 899. For example, the response may include instructions provided to a caregiver interface device 960 and/or medical equipment 170. These responses include, for example, caregiver instructions and/or medical device instructions. For example, the instructions may include an instruction to collect additional data. In certain examples, the AI modeling engine 520 may be configured to abort processing where the probability, or confidence metric, for the output falls below a threshold value. In these examples, the engine 520 may generate a response indicating that the AI modeling engine 520 was unable to understand the last input.

With regard to the channel handler, 1505, each of the contextual data sources 899 may be associated with a channel. Input data received via a channel may specify the source of the inbound communications to the CSG engine 150. Additionally, the channel handler 1505 may direct outbound responses from the CSG engine 150 to the appropriate endpoint(s).

In some examples, to process a request (i.e., inbound unstructured data) the handler 1505 may be configured to generate a communication identifier, store an association between the communication identifier and the channel identifier received in the request, and identify a type of the input data (text, audio, image, etc.) stored in the request. The handler 1505 may be further configured to transmit the communication identifier and the input data specified in the request to the appropriated AI processing sub-engine. In some examples, to utilize a post-processing response, the channel handler 1505 may be configured to identify a channel identifier associated with the communication identifier specified in the response, generate output data based on a response endpoint identified by the channel identifier.

Each of the sub-engines may handle a variety of modeling tasks. The audio sub-processing engine 1510 may include an automatic speech recognition (ASR) engine 1512 and/or an audio classification engine 1514. The ASR engine 1512 may be configured to receive the communication identifier and the audio data from the handler 1505 and to process the same. The ASR engine 1512 renders the text data from the audio data by executing an ASR process (for example, but not limited to, Apple Dictation, Google Gboard, Nuance Dragon Anywhere, Amazon Transcribe, Microsoft Azure Speech to Text, IBM Watson Speech to Text, Windows 10 Speech Recognition, etc.). In an implementation, the ASR engine 1512 may provide rendered text to the NLP processing engine 1520. The ASR engine 1512 uses AI modeling to convert unstructured sounds in the form of speech to probabilistic structured data in the form of text. For example, the ASR engine 1512 may receive the audio input “contusion” and return an output of a text string “c-o-n-t-u-s-i-o-n” with an 87% probability of accuracy and the text string “c-o-n-f-u-s-i-o-n” with a 13% probability of accuracy.

The audio processing engine 1510 may further include an audio classification engine 1514. The engine 1514 may process speech as sounds to generate outputs related to speech volume or speech frequency. Additionally, or alternatively, the engine 1514 may use AI modeling to identify environmental sounds (e.g., siren, gunshot, motor noise, propeller sound, coughing, gurgling, non-verbal screaming, etc.). Environmental sounds, or ambient noise, may also reduce the confidence metric for speech to text conversion by the audio processing engine 1510, and therefore reduce the confidence metric for the output of the NLP processing engine 1520 which may receive converted text from the ASR engine 1512. To mitigate this impact of ambient noise, the microphone hardware 584 may include noise filtering circuitry and/or may include multiple microphones spatially distributed in order to reduce the noise component. In an implementation, a particular environmental sound may result in a clarification response from the audio processing engine 1510. The clarification response may be an instruction for a caregiver to repeat an utterance or an instruction for the CSG engine to repeat an audible instruction and/or increase the volume of the audible instruction.

The NLP processing engine 1520 may include a named entity recognition engine 1522, a text classification engine 1524, a question and answer (Q&A) engine 1526, and a fill-mask engine 1528. In some examples, the NLP engine 1520 awaits a wakeup word to begin applying the natural language processing models to inbound text data. The named entity recognition engine 1522 is configured to extract named entities from text data and classify these entities into predefined categories. The entities may include quantities, percentages, names of people, names of organizations like hospitals, EMS agencies, etc., geographic locations, product names like medical devices, medical supplies, medications, etc., and names of events like care events such as transport, spinal stabilization, intubation, defibrillation, chest compressions, needle decompression, ventilation, fluid administration, etc. The text classification engine 1524 may identify terms and values articulated within the text by applying one or more specialized natural language processing models trained to understand medical terminology, syntax, and grammar utilized by caregivers. The Q&A engine 1526 may be configured to recognize and receive a question from a caregiver, locate relevant sources of material for an answer to the question, extract text for the answer, and return that text as a response. In locating the relevant sources of answer material, the engine applies a confidence metric to each individual source and then selects a predetermined number of sources, N, for which the confidence metric is above a pre-determined threshold. Further the engine extracts the text from the N sources for which the confidence metric exceeds a pre-determined threshold for the answer actually corresponding to the question. The questions and answers for the CSG engine may pertain to the victim(s) demographics and/or medical history, the patient care status for the caregiver crew and/or the victim, transport and transport destination information, medical device and supply availability and usage, and/or context information like weather, traffic, events, etc. The fill-mask engine 1528 is configured to analyze text to mask certain words that either have not been provided or are not decipherable from the speech and then predict the word that fills the mask. Thus, the fill-mask engine 1528 provides predictive speech to fill in blanks, referred to as masks, in the provided text. For example, if the input text for the context of a trauma incident is “the victim has a pneumo <mask>,” the fill-mask engine 1528 may provide “thorax” with an associated confidence metric. As another example, if the input text is “we have bleeding, hand me a <mask>,” the fill-mask engine 1528 may provide “tourniquet” with an associated confidence metric. Thus, the fill-mask engine 1528 may provide mask fill terms and/or phrases according to expected procedural flow for trauma treatment.

The CV processor 1530 may extract structured data by applying one or more specialized image processing models trained to understand images characteristic of a trauma response. For example, the image data may include a photographic image (e.g., a trauma injury, a portion of a victim's body, internal and/or external, an element of the emergency environment, etc.) and/or a medical imaging scan such as an ultrasound scan, etc. In a trauma injury, a camera image may provide evidence of exsanguination and an image analysis may yield an estimated amount of blood loss based on an area identified as blood. In an implementation, the image data may include a series of images that form a video recording. The image recognition engine 1532 may detect, analyze, and interpret images to identify elements of the image according to which the CSG engine 150 may provide responses to the contextual data sources 899. The motion analysis engine 1534 may provide object or person detection, localization, and/or segmentation, motion detection, tracking, and pose estimation. The image reconstruction engine 1536 may enhance raw data input for images to improve image quality adversely affected by a low signal-to-noise ratio, artifacts, a low contrast-to-noise ratio, etc. The images may include medical images like ultrasound images.

The post-processing engines (e.g., the engines 1540, 1550, and 1560), which may also be referred to as data handlers, utilize the structured data probabilistic output from the AI modeling engine 520 to perform an operation of the CSG engine 150. The operation may be an instruction to one or more of the caregivers and medical equipment. The post-processing engines may handle caregiver skill evaluation, environmental data analysis, physiologic data analysis, and transport environment data analysis. This evaluation and analysis may depend on and/or generate clinical findings, caregiver authentication, UI navigation (e.g., for the CSG UI 155), data recordation, image capture, image interpretation, medical device log entries, and data reporting.

In various implementations, the mobile device 110 may be a single device or two or more interconnected devices. To provide audio input to the CSG engine 150, the user may provide the audio input to a device like a watch that is communicatively coupled to the mobile device 110. The mobile device 110 and/or the connected device may record the audio recording and provide the recording to the CSG engine 150 for transcription and processing. The CSG engine 150 may generate the transcription, similarly to the transcription provided to the telemedicine provider, as described herein, and provide the transcription to the caregiver. For example, the CSG UI 155 may include a control to access the transcription. The caregiver may choose to review and/or edit the transcription and then request that the transcription be saved within the ePCR and/or the medical device case file. Alternatively, the CSG engine 150 may automatically store the transcription in one of these files.

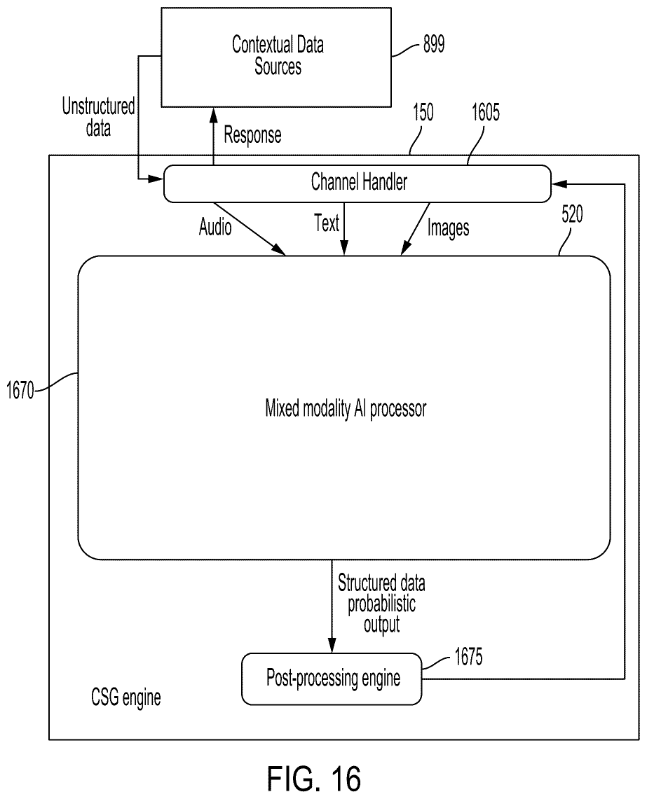

Referring to FIG. 16, a schematic illustration of an example of a mixed modality AI modeling engine of a CSG engine is shown. A quantity of each component in FIG. 15 is an example only and other quantities of each, or any, component could be used. In an implementation, instead of the single mode processing engines for audio, text, and images as shown in FIG. 16 (e.g., the engines 1510, 1520, and 1530), the AI modeling engine 520 may be a mixed modality AI processor 1670. The mixed modality AI processor 1670 may receive all of the audio, text, and image data. The processor 1670 may provide the same functions as the single mode processing engines of FIG. 15. The channel handler 1605 is configured to direct all modes of unstructured data to the mixed modality AI processing engine 1670. The channel handler 1605 may assign channel designations that allow the channel handler 1605 to properly route responses (e.g., as described for the channel handler 1505). Additionally, the post-processing engine 1675 is configured to handle structured data for multiple modes rather than specializing for a particular mode.

Referring to FIG. 17, a schematic illustration of an AI modeling engine with optimized modeling capabilities is shown. Optimized modeling may be of particular importance in locally implementing an AI modeling engine 520 on a smartphone or other device with reduced processing capability as compared to a cloud computing environment. The improved efficiency and accuracy that evolves as the emergency care progresses as provided by the system in FIG. 17 improves both the functioning of the AI modeling engine 520 and the care provided to the patient.

In a cloud computing environment, a large and non-specific general model may apply and execute a potentially unlimited number of sub-models to optimize confidence in the output at least because of large processing and memory capacity available on a cloud server. However, these resources may be more limited on a smartphone as may be the time available for natural language processing in the context of emergency care. Additionally, the cloud server may be unavailable to an emergency scene lacking network connectivity.

The trained model utilized by the AI modeling engine 520 may include, for example, a general model 1710 comprising one or more contextual models (e.g., the contextual models 1700-1705), and/or one or more sub-contextual models (e.g., the sub-contextual models 1760-1769). The general model 1710 may invoke particular contextual or sub-contextual models based on a context and/or a patient care state determined based on the inputs to the AI modeling engine 520. For example, the inputs may be keywords associated with particular models. Thus, rather than following a pre-determined path for invoking the various available AI models, the general model 1710 may orchestrate, direct, and/or coordinate a selection of one or more model(s) applied to the unstructured data input. The context and/or patient care state may progressively change over the course of operations of the AI modeling engine 520 to reflect changes in the caregiver activities, the emergency environment, and/or the patient status. For example, in the case of NLP, vocabulary, syntax, and/or text structure may vary between contexts and the more refined and tailored to the specific context the model may be, the more efficiently and accurately the model may generate the structured text. For instance, one or more of a sentence subject, verb, numerical variables and constants, elements of a medical device text string, etc. may vary in meaning and structure from context to context.

To optimize dynamic model selection, the general model 1710 may evaluate the confidence metric for the structured data to evaluate the model selection. If the confidence is below a certain threshold, the general model may re-select or re-combine various sub-models to re-generate the output and improve the confidence associated with the structured text. The general model 1710 may determine a general context or patient care state and based on this determination, hand off analysis to one or more specific contextual models. In turn, the contextual model may determine a more specific context or patient care state and, based on this determines, hand off analysis to one or more sub-contextual models. Additionally, fast texts, keywords, limited vocabulary sets, and a sentence order may enable an efficient model selection by the AI modeling engine 520. The AI modeling engine 520 may also include physiologic data from the medical equipment 170 to expedite interpretation of other inputs and narrow down a set of NLP models. In an implementation, caregivers may be trained to interact with the CSG engine 150 by using particular keywords in various patient care situations thus enabling the AI modeling engine 520 to efficiently select contextual and sub-contextual models. This training may be agency specific and may incorporate local terminology and usage.

As an example, for CSG, the general model 1710 may hand off to a contextual model for pneumothorax assessment upon a determination of a pneumothorax assessment context. The contextual model for pneumothorax assessment may, upon receipt of unstructured data may identify a sub-context of an ultrasound examination, may hand off to a sub-contextual model for an ultrasound examination as opposed to a sub-contextual model for a stethoscope examination. Additionally, with an identified pneumothorax assessment context along an external bleeding intervention context, the general model 1710 may invoke specific combinations of contextual models to handle the specific mix of unstructured data.

In some implementations, the model selection may occur on demand at the point of care and/or may be previously provisioned. For example, the general model 1710 may be provisioned to utilize particular sub-models for patient conditions and/or EMS operations typically seen by a particular agency or transport crew. Additionally, the general model 1710 may be configured to recognize an unexpected patient condition and/or EMS operations and identify and utilize a different sub-model.

The contextual and sub-contextual models may reflect specific contexts in terms of at least geo-location, modality of care, protocols, historic patterns of care, type of EMS service, a type or nature of service, etc. For example, the contextual models for trauma care may vary from a northern climate where a victim may be likely to be positioned on a frozen ground surface to a southern climate where such a surface may be unusual. As another example, the contextual models may be different for a small rural EMS agency that primarily deploys a few helicopters as opposed to a large urban EMS agency with a fleet of ambulances. As a further example, the context of a military emergency scene may be different and require a different contextual model than the context of a call to a highway car collision. The geo-locations of the responders and/or the transport vehicles in these two situations may enable a distinction between these two contexts.

In order to dynamically improve modeling efficiency, the AI modeling engine 520 may utilize the structured output and associated confidence metric for a first set of unstructured input to predict, anticipate and/or prioritize the next contextual, and optionally, sub-contextual, models for the next or a subsequent set of unstructured input.

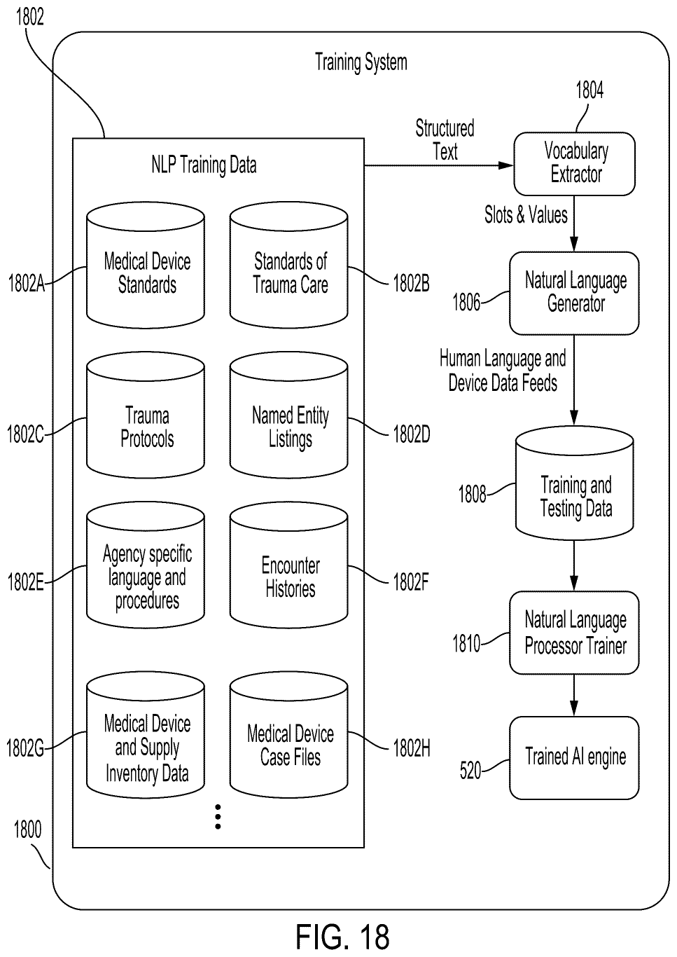

Referring to FIG. 18, an example of a data flow diagram illustrating an NLP training system and process for a conversational user interface in accordance with examples of the present disclosure is shown. The training system 1800 processes a variety of data to train one or more NLP AI models as described herein. For example, the data may be from data stores including, but not limited to, one or more of medical device standards 1802A, standards for trauma care 1802B, trauma protocols 1802C, named entity listings 1802D, agency specific language and procedures 1802E, EMS encounter histories for victims 1802F, medical device and supply inventory data 1802G, medical device case file data 1802H, etc. As shown in FIG. 18, the system 1800 further includes a vocabulary extractor 1804, a natural language generator 1806, an NLP trainer 1810, and the trained AI modeling engine 520. In some examples, the system 1800 may be implemented using a server environment, such as the cloud server 375 of FIG. 5, although implementation via less powerful computing devices may be possible. Each of the data stores 1802A-1802G may be curated sources of structured text data that may be used to build training and testing data housed within the training and testing data store 1808. This training and testing data specifies natural language communications that use the medical terminology, syntax, and grammar of caregivers. The medical device standards data store 1802A includes vocabulary, device settings, physiologic data, and procedure sequences associated with medical devices typically deployed for trauma interventions. The standards of trauma care data store 1802B includes vocabulary, trauma intervention standards such as conditions and recognized interventions, medicine dosages, order of operations, risk to life evaluations of conditions, etc. The trauma protocols data store 1802C includes the vocabulary and instructions of the protocols shown in FIG. 9B that are accessible by the protocol loading engine 515. The named entity listings data store 1802D includes named entities associated with one or more EMS agencies. This data store may be specific to a particular agency or group of agencies and/or may include geographically specific data. The agency specific language and procedures data store 1802E may include vocabulary, procedures, and protocols specific to a particular agency or group of agencies and/or may include geographically specific data. The encounter histories data store 1802F may include historic information for encounters between EMS and victims. This data store may indicate patterns of behavior and/or typical sequences of behavior and associated vocabulary. The medical device and supply inventory data store 1802G includes vocabulary for medical devices and supplies typically associated with a transport vehicle or caregiver crew responding to a trauma event. The data store 1802G may also include data for combination of medical devices, devices and supplies, use of the devices and supplies, and caregiver skill levels required for these devices and supplies. The medical device case file data store 1802H includes vocabulary, phrases, and sentences included in a medical device case file. This data may include JSON format data stream information.

Continuing with the system 1800, the system may use intent classification and slot values to provide a conversational interface however this is only an example and not limiting of the disclosure. The vocabulary extractor 1804 may be configured to retrieve and process structured text data from each of the data stores 1802A-1802E. As an example of a training method, the vocabulary extractor 1804 may to extract slots and slot values from the text data. The slots and slot values enable the system to use an intent classifier to match human words to a pre-determined intent and extract contextual details by leveraging slot filling. When an intent is classified by the system, the slot-filling system directs the user to input words that the AI modeling engine 520 is trained to recognize to fill pre-defined slots. For example, if the first responder asks, “What is the distance to Mercy Hospital?,” based on the training, the system will match “distance” to a pre-defined intent category like “find distance” and then fills an entity “Mercy Hospital” into a slot designated for “destination.” Thus, the intent of “find distance” may be applied to other hospitals because any of these can fill the slot of “destination.” As another example, the caregiver may say “turn on the ventilator.” The intent to power on a ventilator can be derived from this utterance. However, the system must also determine from other parts of the caregiver's speech when (e.g., immediately, in 3 minutes, etc.) and how (e.g., ventilator settings when powered on).

The vocabulary extractor 1804 may maintain a list of formats utilized by each of the data stores 1802A-1802H and processes text data retrieved from each data store using its associated format. In this way, the vocabulary extractor 1804 may consistently extract slots and slot values from the text data retrieved from each of the data stores 1802A-1802H.

Continuing with the system 1800, the human language generator 1806 may be configured to receive the slots and slot values for the unstructured data from the vocabulary extractor 1804 and generate conversational human language communications. For example, where vocabulary extractor 1804 passes a blood pressure slot having a value of 120/80, the human language generator 1806 may construct a sentence such as, “The patient's blood pressure may be 120/80.” Next, the human language generator annotates each of the generated human language communications with labels indicating its associated intent, slot(s), and slot value(s) and stores these annotated communications in the data store 1808 for subsequent processing.

Continuing with the system 1800, the natural language processor trainer 1810 may be configured to train one or more NLP models that make up the trained NLP engine 1520. In some examples, the trainer 1810 retrieves a portion of the annotated human language communications from the data store 1808 and trains one or more NLP models by executing a training process (e.g., stochastic gradient descent) using the retrieve data. In some examples, the NLP models may be models based on a data science and machine learning framework, such as, but not limited to, TensorFlow, Brain, Keras, Apache MXNET, etc. Once the models may be trained, the natural language processor trainer 1810 tests the trained models to determine accuracy. Where the accuracy transgresses a required threshold, the trainer 1810 publishes the models, which become a trained NLP for production use (e.g., as the trained NLP engine 1520).

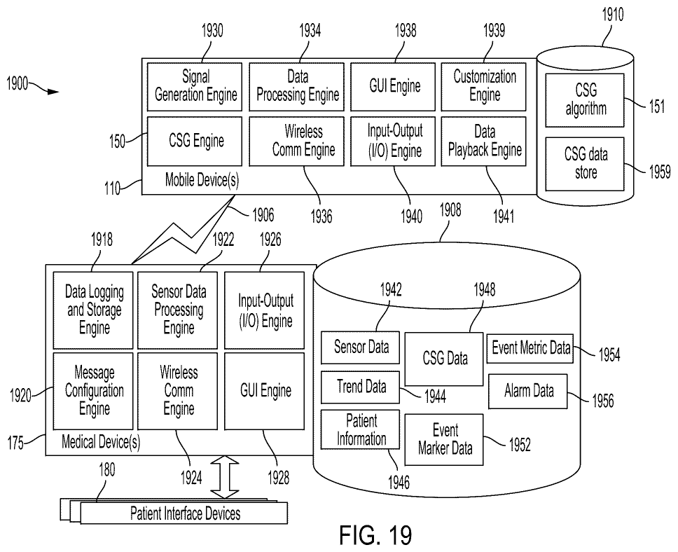

Referring to FIG. 19, an example environment for implementing CSG at a mobile device is shown. This environment includes a medical treatment system with a set of devices for providing treatment and/or interventions to the patient during a medical event. The system may include the medical equipment 170 and one or more mobile devices 110 communicatively coupled to the medical equipment 170 via communication link 1906. The communication link 1906 may include the communication link 199 shown in FIG. 3A and/or the communication link 399 shown in FIG. 5. The devices 110, 170, in the illustrated example, may be co-located at a patient care site. In other implementations, one or more computing devices 310 may be located remotely from the patient care site as illustrated in FIG. 5. For the remote device, the communication link 1906 may include a network connection via the network 380. The devices may be configured for data communication in a wired or wireless manner for transferring information between certain devices 110, 170 of the system during and/or subsequent to delivery of therapy and other interventions.

In some implementations, the wireless communication link 1906 for connecting the medical equipment 170 and the mobile device 110, in some examples, may be a Wi-Fi network, other short-range wireless communication network or near field communication (NFC) network, local area network (LAN), wide area network (WAN), or the Internet. In other examples, the devices 110, 170 can be configured to communicate over longer communications ranges such as over a cellular communication network. In some implementations, the medical equipment 170 may function as a wireless access point to provide a direct wireless connection with the mobile device 110. In other examples, the wireless communication link 1906 can be provided via Bluetooth personal area network.

In some embodiments, different devices 110, 170 may be configured to communicate with one another over different types of communication links 1906. In some implementations, the devices 110, 170 can be configured to transmit data via a short-range wireless communication transmitter, e.g., a Bluetooth beacon, to a receiver. In one example, a first computing device may communicate with the medical equipment 170 via a Wi-Fi and/or cellular communication link from a remote location while a second computing device may communicate with the medical equipment 170 via a Bluetooth communication link. In some implementations, a mobile device 110 can connect to the medical equipment 170 via the wireless communication link without having to physically access the medical equipment 170. In some examples, transport layer security (TLS) is used at an application level to provide a secure (encrypted) connection between the devices 110, 170. As a second layer of protection, encrypted Wi-Fi or encrypted Bluetooth can be used at a physical layer.

In some implementations, when the wireless communication link 1906 is a cellular communication link, the functionality of the medical equipment 170 can be extended to clinicians who are off-scene and/or performing remote telemedicine (e.g., the caregiver 389 at a remote location as shown in FIG. 5). For example, when EMS are transporting a patient to the hospital in an ambulance, a medical team awaiting the arrival of the patient to the hospital can stream real-time case information at a mobile device 110 at the hospital via cellular link. In some examples, the wireless communication link 1906 can include combinations of multiple wireless communication networks based on proximity of the medical equipment 170 to the mobile device 110.

In some implementations, each of the medical equipment 170 and the mobile device 110 includes a respective wireless communication engine 1924, 1936 for enabling wireless communication between the devices 110, 170 via the wireless communication link 1906. For example, the wireless communication engine 1924 of the medical equipment 170 can be configured to transmit messages generated by message configuration engine 1920 to the mobile device 110. Wireless communication engine 1936 of the mobile device 110 can be configured to transmit instruction signals generated by signal generation engine 1930. In some examples, such instruction signals may be for controlling one or more functional operations of the medical equipment 170. In some examples, the wireless communication engines 1924, 1936 are configured to apply encryption protocols to outgoing and transmitted signals. Similarly, the wireless communication engines 1924, 1936 can decrypt incoming and received signals.

In certain embodiments, the wireless communication engine 1924 of the medical equipment 170 can be configured to detect that a respective mobile device 110 is within communication range and in response, initiates one or more actions to connect to the mobile device 110 via the wireless communication link 1906. In some implementations, a mobile device 110 that is pairable with the medical equipment 170 can be preconfigured as a companion device to automatically connect to the medical equipment 170 via a wireless communication link when within communication range, without having to discriminate between other devices that happen to be within range and/or negotiate a wireless communication connection. Further, rather than requiring a user to potentially spend significant amounts of time in manually configuring the system of each companion mobile device to connect to the medical equipment 170 or accessing a screen to view and then select from possible device connections, companion mobile device(s) located at the emergency scene may be pre-configured to dynamically join and/or leave the secure network or pairing with the medical equipment 170, for example, automatically and/or with one or more simple actions (e.g., switch actuation, pressing a button, near field communication connection, radio frequency, location/proximity recognition, gestural code, tap/bump recognition, motion-activated, sound/vibration, voice command/recognition, amongst others) and/or merely by being in close physical proximity to one another such as by a Bluetooth proximity connection. For example, upon selecting the device search control 1426, as shown in FIG. 14A-1, a device user may view all available pre-configured wireless communication links that are available for the mobile device 110 to connect to the medical equipment 170. The user can also view other available networks that have not been pre-configured for connection. In some examples, the companion mobile device may be pre-configured for pairing to other medical treatment devices, and those preconfigured networks can also be displayed upon selecting the device search control 1426.

Once such connection via the wireless communication link 1906 is made, despite the presence of numerous other devices located nearby, patient information (e.g., physiologic data, patient history, rescue info) can be sent back and forth between the connected devices 110, 170 in a reliable and secure manner (e.g., according to HIPAA standards, 802.11i protocols) using any suitable type of communication. Versions of the mobile device 110 that are correctly paired with their respective medical equipment 170 can help avoid risk of erroneous patient information to be transmitted between medical devices, which could be detrimental to patient outcomes. In some embodiments, to maintain accurate and secure communications, the proximity-based interaction may invoke an authentication protocol, such as the use of encrypted keys, vector initialization, hash encryption, digital certificates, etc., ensuring no drops and/or leakage of data transfer between devices. Additionally, the wireless communication engine 1924 of the medical equipment 170 can be configured to simultaneously cause transmission of real-time streaming data to multiple mobile devices or other computing devices via separate wireless communication links 1906 for each mobile device or other computing device.

In some implementations, a number of additional security-oriented design elements can also be implemented for the medical treatment system to ensure that data exchanged between the medical equipment 170 and mobile device 110 remains secure. For example, the medical equipment 170 and/or the mobile device 110 can use certificate-based authentication to ensure the authenticity of the respective paired device. In some examples, upon initial connection and setup, the devices 110, 170 can execute an association process to tie a particular mobile device 110 to a single medical equipment 170 to limit device connections such that only the particular mobile device (and any other similarly paired mobile devices) can interoperate with the medical equipment 170. In some embodiments, any Representational State Transfer (REST) or WebSocket communications may require an authenticated connection to enable data exchange between the devices 110, 170. The medical equipment 170, in some examples, prohibit connection to open Wi-Fi communication links and may only connect to manually defined (e.g., supervisor-defined) Wi-Fi networks. As an additional security measure, when the wireless communication link 1906 is a Bluetooth connection, the devices are paired during initial setup when initial connection settings are configured. Further, the data and computer architecture of the medical equipment 170 can be designed for additional security, which can include separating communications and clinical control onto separate microprocessors.

In some examples, when more than one mobile device 110 is paired with the medical equipment 170, one of the mobile devices may be designated in advance as the primary mobile device. The primary mobile device may be so designated by the medical equipment 170 during device setup, pairing and provisioning by receiving and storing an encrypted token from the medical equipment 170. The encrypted token may be sent with every instruction from the primary mobile device to the medical equipment 170.

In some implementations, the mobile device 110 includes a signal generation engine 1930 that generates instruction signals, data requests, and other data signals for transmitting to the medical equipment 170. In some examples, in response to receiving user inputs (e.g., selections at a touchscreen of the mobile device 110) to view one or more items of case information at customized user interface screens on the mobile device 110, the signal generation engine 1930 can generate a data request message from transmitting to the medical equipment 170. In some examples, the data requests can be of one or more data requests type based on a type and amount of data being requested. For example, one type of data request includes a request for a single type of data from the medical treatment device (e.g., trends data) or for a relatively small number of pieces of data (e.g., ventilation data for generating a ventilation dashboard). Another type of data request can include requests for complex data groupings, such as all case information necessary to recreate a device view or requested case type view. Generating specific data requests of the medical equipment 170 allows the mobile device to flexibly modify the set of data it has subscribed to at any moment. In some examples, data subscriptions for the mobile device 110 correspond to all of the data requested by the mobile device 110 for display at any given time. Requesting data from the medical treatment device as a set of data subscriptions provides mobile device 110 the ability to show different types of data on demand and minimizes bandwidth used by avoiding transmission of unnecessary data that the mobile device user does not wish to have displayed.

In certain embodiments, the signal generation engine 1930 can also generate instruction signals for causing one or more functional operations to occur at the medical equipment 170. In some examples, the one or more functional operations can include linking and storing certain submitted data associated with the medical event (e.g., patient information or event marker data) and/or capturing or analyzing certain medical event data (e.g., generating 12-lead ECG analysis, lung mechanics data analysis, ultrasound image analysis, etc.). For example, the signal generation engine 1930 can generate a patient information signal in response to receiving submission of patient identification information at the mobile device 110. The patient information signal can include the submitted patient information, and in response to receiving the signal, the medical equipment 170 can link and store received patient information 1946 with sensor data 1942 and other case information for the patient within data repository 1908. In addition, in response to receiving one or more event marker inputs at the mobile device, the signal generation engine 1930 can generate an event marker signal, which can include submitted event marker data. In response to receiving the signal, the medical equipment 170 stores the submitted event marker data 1952 in data repository 1908. In some examples, the signal generation engine 1930 can generate an instruction signal that causes the medical equipment 170 to perform a particular data analysis, send specific data to the mobile device, and/or administer a particular therapy.

In some implementations, the medical device(s) may store alarm data 1956 in data repository 1908. The alarm data 1956 may include alarms provided by the medical device(s). The alarm data 1956 may indicate alarm information such as type, priority, time, etc. Types of alarms may include patient safety alarms, use environment alarms, and/or self-check alarms. The alarm priorities may be one of “high,” “medium,” and “low” or a priority according to another rating scheme (e.g., severity indicated as numbers or letters) based on the severity of the associated alarm. In some examples, the medical device(s) may categorize all patient safety alarms as high priority alarms.

The medical equipment 170, in some implementations, can include a message configuration engine 1920 for generating messages for sending to the mobile device 110. In one example, in response to receiving a data request from the mobile device 110, the message configuration engine 1920 can package the requested data in one or more predetermined message configurations or formats for transmission. In some implementations, real-time or near real-time data (e.g., case information derived from patient interface devices 180) can be transmitted as streaming data in a JavaScript Object Notation (JSON) format sent over a WebSocket. Historical and bulk data transfers, in some examples, can be transmitted as Representational State Transfer (REST) data in JSON-formatted messages. Both types of message communications (streaming data and REST data) can occur over a transport layer security (TLS) connection, which can use a TCP/IP protocol. The TCP/IP protocol can be provided over Wi-Fi or Bluetooth physical media. When data transfer occurs in real-time or near real-time, case information is simultaneously displayed at the mobile device 110 and the medical equipment 170 or an amount of latency for displaying the case information at the mobile device is less than a predetermined threshold.

In addition to configuring messages for sending real-time case information for display at the mobile device 110 in some implementations, the message configuration engine 1920 can generate one or more confirmation messages when an action is taken at the medical equipment 170 in response to receiving an instruction signal from the mobile device 110. For example, in response to associating and storing submitted patient information provided by the mobile device 110 the message configuration engine 1920 can generate a message confirming that the submitted patient information has been linked to the respective case information within data repository 1908. The message configuration engine 1920 can also generate event marker recording confirmation messages confirming that recording of the submitted event marker at the medical equipment 170 has commenced and/or completed.

In some implementations, the mobile device 110 may include a data playback engine 1941. The data playback engine 1941 can provide caregivers the ability to view and scroll through past case information at the mobile device 110. In some examples, while viewing past case information, the mobile device 110 can provide users the ability to jump to a current (live) view at any time during the medical event. In one example, the mobile device 110 may indicate on the display screen whether a live view is being displayed. In some embodiments, mobile device users can jump forward and backward in time in discrete time segments (e.g., 5, 10, 15, 20, 30 seconds) to view a patient's physiological condition and caregiver performance data at different points during the medical event. In one example, the mobile device display interface can provide a touch spot for each waveform that provides for playback of the respective waveform. Additionally, the mobile device 110 can play back past medical event data at different speeds (e.g., 0.5×, 1×, 2×) to gain a better perspective of how patient conditions and care have progressed. In some examples, the mobile device can display past medical data for a number of different monitored physiological sensor inputs (e.g., ECG, SpO2, EtCO2, etc.), vital sign data, and caregiver performance data. Additionally, even when non-real-time data is being displayed at the mobile device 110, the medical equipment 170 continues to transmit real-time streaming case information to the mobile device 110. In one example, the mobile device 110 can also provide a lookback feature for limited time increments (e.g., 10, 20, 30 seconds) that allows the user to quickly view waveform data at the lookback increment. In example, the lookback feature can be activated via a touch spot on the respective waveform.

In some implementations, the medical equipment 170 can include an input/output (I/O) engine 1926 that may gather information from a number of patient interface devices 180 built into the medical equipment 170 and/or in communication with the medical equipment 170. The I/O engine 1926 can also receive user inputs at a user input interface (e.g., keypad or touchscreen) on the medical equipment 170. In some examples, raw sensor data from the patient interface devices 180 can be processed and analyzed by sensor data processing engine 1922, which generates a number of clinical metrics and trends regarding aspects of the treatment session (e.g., for use by clinical personnel) that can be output by the medical equipment 170 (e.g., displayed at a screen of the medical equipment 170 or printed as a report). The generated metrics and trends can be stored in data repository 1908 for the respective patient and/or medical event as metric data 1954, and trend data 1944, respectively. GUI engine 1928, in some embodiments, causes data processed and analyzed by the sensor data processing engine 1922 to be presented at a display interface screen on the medical equipment 170.

The medical equipment 170 may include a data logging and storage engine 1918. The engine 1918 may enable storage of the various types of medical device data shown in FIG. 19 in the data repository 1908. The engine 1918 may log the data as it is generated by or received at the medical equipment 170, associated a time stamp with the data, and then store the logged data in the repository 1908.

In some examples, sensor data processing engine 1922 also processes raw sensor data into first sensor information that is provided to GUI engine 1928 for display at medical equipment 170 and second sensor information that is provided to GUI engine 1938 for display at mobile device 110. In some examples, because the display interfaces at the medical equipment 170 and mobile device 110 can be differently sized, the sensor processing engine 1922 can time-slice the sensor data differently based on the display device. In one example, the local display for the medical equipment 170 receives small intervals of first sensor information more frequently (e.g., 8 ms at a time) while the mobile device 110 receives larger intervals of second sensor information less frequently (e.g., 120 ms at a time). This configuration of first and second sensor information can allow the mobile device 110 to display sensor data in real-time yet also improves data transmission efficiency because sending larger data messages is more efficient than sending smaller data messages. Additionally, even though the content of the first and second sensor information is the same, the first and second sensor data may be represented differently. For example, the first sensor information displayed at the medical treatment device may include binary data records while the second sensor information the mobile device 110 receives may be a JSON representation as a Websocket stream. Additionally, both the first and second sensor information signals can carry additional information per-sample bit-flags that represent specific detected conditions associated with the processed sensor data sample. Other additional information, such as medical device settings may be provided to both of the GUI engines 1928, 1938 as additional messages separate from the sensor data.

In some aspects, a data processing engine 1934 of the mobile device 110 can be configured to process sensor data 1942 and other case information received from the medical equipment 170 in one or more received data messages. GUI engine 1938 can cause the processed medical event data to be displayed in one or more display sections of a device interface at the mobile device 110. An I/O engine 1940 can receive and process user inputs at a device interface, such as a touchscreen interface, where a user makes selections to customize the display of data at the mobile device 110 as well as to cause one or more functional operations to occur at the medical equipment 170. GUI engine 1938, in some implementations, causes data processed and analyzed by the data processing engine 1934 and configured by customization engine 1939 to be presented at a display interface screen on the mobile device. Customization engine 1939, in some embodiments, can be configured to control and manage the configuration of display screens presented at the display interface of the mobile device 110 by the GUI engine 1938 based on a user role and/or data presentation preferences. Roles may include, for example, chest compression team member, ventilation team member, drug administrator, supervisor, documenter, remote clinicians, or non-clinicians (e.g., police and/or fire personnel as first responder(s)).

The mobile device may further include the CSG engine 150. A data store 1910 associated with the mobile device 110 may include a CSG data store 1959 and a CSG algorithm 121 and/or a CSG application if the cloud server hosts the CSG engine 150. In an example, the CSG data store 1959 may be a trauma CSG data store with data specifically tailored to trauma care. The CSG data store 1959 may include trauma CSG data provided to the mobile device 110 by the user and/or by the medical equipment 170. The data storage region 1908 of the medical equipment 170 may include CSG data 1948 received from the mobile device 110 and/or collected by or generated at the medical equipment 170.

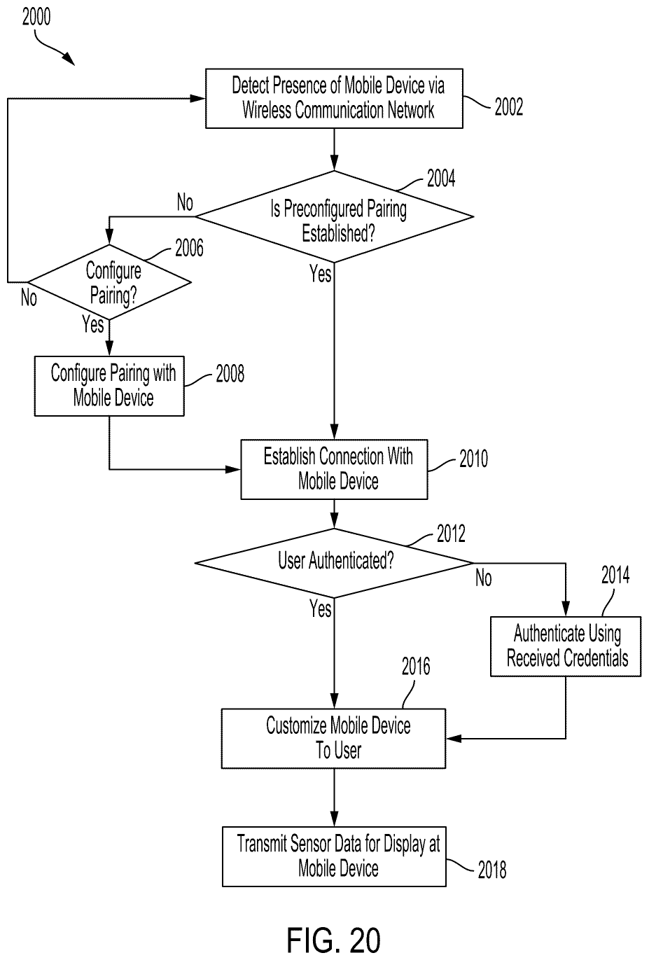

Referring to FIG. 20, an example method for configuring connection between a medical device and a mobile device is illustrated. In response to the connection, the connected devices window 1420 may display an indication of each device that is communicatively coupled to the mobile device 110. In some examples, the method 2000 begins with medical equipment 170 detecting the presence of the mobile device 110 due to the mobile device 110 being within communication range of the medical equipment 170 (2002). If the medical equipment 170 and the detected mobile device 110 already have a preconfigured pairing with each other established (2004), then in some examples, the medical equipment 170 and the mobile device 110 establish a wireless connection (2006). In some embodiments, the previously paired medical equipment 170 and the mobile device 110 may automatically connect to one another. In other examples, upon detection of the respective device, the medical equipment 170 and/or the mobile device 110 may present a notification to a user at a display interface requesting user authorization for connection to the other device 110, 170. In response to presenting the notification, if one or both of the devices 110, 170 receives an input confirming the connection to the other device 110, 170, then the wireless communication link between the devices 110, 170 is established.

If a preconfigured pairing between the two devices 110, 170 has not been established, then in some aspects, a determination may be made whether to establish a wireless connection between the devices 110, 170 (2006). In some examples, only devices 110, 170 that have been through an initial setup and pairing process can wirelessly connect to one another. In another example, a supervisor or system administrator may be authorized to configure a pairing between the medical equipment 170 and the mobile device (2008) prior to initial connection between the devices 110, 170 (2010). In one example, this initial pairing can include one or more types of wireless communication links such as Wi-Fi, Bluetooth, or Zigbee connections.

In certain embodiments, if a credentialed user has been authenticated at the mobile device (2012), then in some examples, the mobile device 110 may customize one or more mobile device display views to user preferences or a treatment role of the user (2016) based on role data, as stored or provided at the mobile device at time of use. In some examples, if less than a threshold amount of time has passed between uses of the mobile device 110, the mobile device 110 may automatically authenticate a previous user to use the mobile device 110. Otherwise, if more than the threshold period of time has passed and/or a new user is logging in to use the mobile device, then in some implementations, the mobile device 110 may authenticate the user with received credentials, such as username/password inputs, at least one biometric input provided at a biometric input interface (e.g., fingerprint, iris recognition, facial recognition), and/or a badge scan via a scanning sensor on the mobile device (e.g., RFID scan or a computer-readable code such as a QR code) (2014). In some examples, upon authentication, the user may provide one or more inputs confirming or modifying a treatment role of the user in the associated medical event. Based on the indicated treatment role, the mobile device 110 may further customize the display views at the mobile device 110 to the indicated role of the user. In some embodiments, based on the customized display interface screens being presented at the mobile device as well as user inputs received at the display interface, the medical equipment 170 may transmit requested sensor data for display at the mobile device in the customized format (2018). In some implementations, in response to establishment of the connection between the mobile device 110 and medical equipment 170 and/or authentication of the device user, the medical equipment 170 may automatically begin streaming case information via the wireless communication link 2406 for display at the mobile device 110 without any user intervention.

Although described as a particular series of steps, in other embodiments, more or fewer steps may be included. For example, the medical equipment 170 may only connect to a mobile device 110 that has had a preconfigured pairing established. On the other hand, if it is determined that a predetermined pairing exists between the medical equipment 170 and a detected mobile device 110, one or more additional steps related to ensuring data security of the wireless connection may be performed. In further embodiments, certain steps may be performed in a different order, or two or more steps may be performed in parallel. For example, a user may be authenticated at the mobile device 110 prior to a connection being established between the medical equipment 170 and the mobile device 110. Other modifications of the method 2000 are possible while remaining in the scope and purpose of the method 2000.

Referring to FIG. 21, an example method for causing display of case information from a medical device at a mobile device during a medical event is illustrated. The method 2100 is, however, an example only and not limiting. The method 2100 can be altered, e.g., by having stages added, removed, rearranged, combined, and/or performed concurrently. The method 2100 enables a display of the device view window, the working view window, and the trend view window as discussed above in regard to FIG. 14A-1.

In some examples, the method 2100 begins with mobile device 110 transmitting a device view instruction signal (2102) to connected medical equipment 170. In some implementations, the instruction signal corresponds to a request for case information that is presented within the device display view at the mobile device 110. In some examples, the device view is one of multiple display views that can be presented at the mobile device 110 and corresponds to a display view that, in real-time, presents a visual reproduction of the data that is presented at a display interface of one or more of the medical equipment 170. In some embodiments, in response to transmitting the request signal for device view case information, the medical equipment 170 sends the requested data over the wireless communication link, which is received by the mobile device 110 (2104). In some examples, the mobile device 110 causes display of the received information within the device view at the display interface (2106).

In some implementations, upon detection selection of a trends view input (2108) at the mobile device 110 (e.g., selection of trends view selector 1414), the mobile device may configure the case information to cause display of a trends view display interface at the mobile device (2110). In some implementations, configuring trend data for display at the trends view display interface may include transmitting a signal to the medical equipment 170 to request transmission of medical trend data for real-time display.

Upon detection of a working view activation input (2112) at a display interface (e.g., working view selection tab 1412), the mobile device may configure the case information to cause display of a working view display interface at the mobile device (2114). In some examples, the working view display interface may be activated when the user configures the mobile device to remove or add any displayed data section (e.g., waveform, data value, or dashboard). For example, the user may select data sections to delete or include in real time at the mobile device 110. In other examples, a saved working view associated with a user account may be automatically presented upon logging in to use the mobile device 110. In some embodiments, the working view window can also be activated upon addition of any waveform, metric value, or dashboard for viewing. In some embodiments, the working view display interface may correspond to any type of customization of the case information displayed at the mobile device. Stated another way, the working view display interface corresponds to any set of displayed data in any format at the mobile device 110 that differs from the displayed data and format of the device view.

In some implementations, upon configuration of a working view screen at the mobile device, if one or more items of physiological sensor data, treatment data, or caregiver performance data are needed to display within the working view (2116), then in some examples, the mobile device 110 transmits a data acquisition request to the medical equipment 170 requesting the one or more requested items of data (2118). For example, if a mobile device user selects a chest compression dashboard for display within a working view at the mobile device 110 and it has not been displayed up to that point, then the mobile device may transmit a data acquisition request to the medical equipment 170 to obtain CPR case information (e.g., chest compression depth and rate data) for display within the chest compression dashboard. In some examples, upon receiving the requested data, the mobile device 110 configures the requested data for real-time display within a respective data section in the working view interface (2120). In some embodiments, based on the type of data requested, the medical equipment 170 may transmit the requested data as a streaming data message and/or a REST data message (bulk data transfer).

Referring to FIG. 22, examples of components of various devices discussed herein are shown schematically. These devices may include a medical device 2202 (e.g., as shown in FIG. 2D) and one or more mobile devices 2204 (e.g., mobile device 110 in FIG. 2A and/or mobile devices 110a and 110b in FIG. 6). The mobile device 2204 may include a user interface 2229 (e.g., a touchscreen or other display device and/or audio output device) and/or the medical device may include a user interface 2219 (e.g., a touchscreen or other display device and/or audio output device). In an implementation, the medical device 2204 may monitor and/or provide diagnostic care for the patients 101a and/or 101b and/or provide medical therapy as an intervention via the patient interface devices 2230 (e.g., the patient interface devices 180). The mobile device 2204 may be a portable computing device such as a tablet, laptop, smart-phone, watch, heads-up device, or combination thereof. The mobile device 2204 may be adapted to function as a medical device or be a display screen of an additional medical device such as when monitoring continuous NIBP measurements. In an implementation, the mobile device 2204 may not be a therapeutic medical device configured to deliver medical therapy to the patient. In such an implementation, the mobile device 2204 may be limited to patient monitoring and/or diagnostic care, such as tracking a patient status via one or more display screens and/or controlling one or more functional operations at the medical treatment device 2202 as described in the embodiments herein.

The medical device 2202 and one or more mobile devices 2204 may be communicatively coupled via communicative coupling 2206 (e.g., the communication link 399 as shown in FIG. 5), which may be a wired and/or a wireless communications link. The wired communications links may include a wired electrically coupling, an optical coupling via an optical cable, etc. The wireless communications link may include coupling via a radio frequency or other transmission media and/or via a network such as a local area network, an ad hoc network, a mesh network, a cellular and/or another communications network, a computer network, etc. The communications link as described herein may utilize protocols such as, for example, 802.11, ZigBee®, Bluetooth®, etc. The communications link may include near field communications which may be implemented via a communications RFID tag. The communications link may include one or more networks such as a local area network, a cellular network, a satellite network, and/or a computer network (e.g., an Internet Protocol (IP) network). In various implementations, the communicative couplings described herein may provide secure and/or authenticated communications channels. In an implementation, the devices described herein may encrypt and/or decrypt the data transmitted and/or received via the communicative couplings.

In some embodiments, the memory 2210 of the medical treatment device 2202, and similarly memory 2222 of mobile device 2804, may include a data store, also referred to as a data repository, and corresponding data storage circuitry including one or more of non-transitory or non-volatile computer readable media, such as flash memory, solid state memory, magnetic memory, optical memory, cache memory, combinations thereof, and others. The data store can be configured to store executable instructions and data used for operation of the medical treatment device 2202 and/or mobile device 2204. In certain implementations, the data storage processing circuitry, in combination with the data store, can include executable instructions that, when executed on the processor 2208 and/or 2220, are configured to cause the at least one processor 2208 and/or 2220 to perform one or more functions of the medical device(s) 2202 and the mobile device 2204, as described herein.

The engines described herein (e.g., the various engines shown in FIGS. 2A, 2B, 3A, 3E, 4, 5, 8A, 8B, 9A, 9B, 15, 16, 17, 18, and 19) may include hardware logic and/or software logic provided by one or more processors and/or memory to implement logic instructions to perform one or more tasks at one or more computing devices and/or at one or more medical devices. In some examples, the actions performed by engines may be implemented by hardware, software, firmware, middleware, microcode, hardware description languages, etc., or any combination thereof. When implemented in software, firmware, middleware, or microcode, the program code or code segments to perform the tasks may be stored in a non-transitory processor-readable medium such as a storage medium. Processors may perform the described tasks. In various implementations, the engines described herein may be or may include at least one processor and a non-transitory processor-readable storage medium having stored thereon processor-readable instructions configured to cause the at least one processor to perform actions according to the processor-readable instructions. The storage medium and/or the processor may be components of a computing device and/or a medical device.

In FIG. 22, the components 2208, 2210, 2212, 2214, 2216, 2218, and 2219 are communicatively coupled (directly and/or indirectly) to each other for bi-directional communication. Similarly, the components 2220, 2222, 2224, 2226, 2228, and 2229 are communicatively coupled (directly and/or indirectly) to each other for bi-directional communication.

In some implementations, the components 2208, 2210, 2216, and/or 2218 of the medical devices 2202 may be combined into one or more discrete components and components 2216 and/or 2218 may be part of the processor 2208. The processor 2208 and the memory 2210 may include and/or be coupled to associated circuitry in order to perform the functions described herein. Additionally, the components 2220, 2222, and 2228 of mobile device 2204 may be combined into one or more discrete components and component 2228 may be part of the processor 2220. The processor 2220 and the memory 2221 may include and/or be coupled to associated circuitry in order to perform the functions described herein.

In some implementations, the medical device 2202 may include the therapy delivery control module 2218. For example, the therapy delivery control module 2218 may be an electrotherapy delivery circuit that includes one or more high-voltage capacitors configured to store electrical energy for a pacing pulse or a defibrillating pulse. The electrotherapy delivery circuit may further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 2218 may be a compression device electro-mechanical controller configured to control a mechanical compression device. As a further example, the therapy delivery control module 2218 may be an electro-mechanical controller configured to control drug delivery, temperature management, ventilation, and/or other type of therapy delivery.

The medical device 2202 may incorporate and/or be configured to couple to one or more patient interface devices 2230. The patient interface devices 2230 may include one or more therapy delivery component(s) 2232a and one or more sensor(s) 2232b. Similarly, the mobile device 2204 may be adapted for medical use and may incorporate and/or be configured to couple to one or more patient interface device(s) 2234. The patient interface device(s) 2234 may include one or more sensors 2236. The sensor(s) 2236 may be substantially as described herein with regard to the sensor(s) 2232b.

The sensor(s) 2232b and 2236 may include sensing electrodes (e.g., the sensing electrodes 2238), ventilation and/or respiration sensors (e.g., the ventilation and/or respiration sensors 2240), temperature sensors (e.g., the temperature sensor 2242), chest compression sensors (e.g., the chest compression sensor 2244), and an ultrasound sensor (e.g., the ultrasound sensor 2246), etc. In some implementations, the information obtained from the sensors 2232b and 2236 can be used to generate information displayed at the medical device 2202 and simultaneously at the display views at mobile device 2204 and described above (e.g., at the various UI view windows in FIG. 14A-1). In one example, the sensing electrodes 2238 may include cardiac sensing electrodes. The cardiac sensing electrodes may be conductive and/or capacitive electrodes configured to measure changes in a patient's electrophysiology to measure the patient's ECG information. The sensing electrodes 2238 may further measure the transthoracic impedance and/or a heart rate of the patient. The ventilation and/or respiration sensors 2230 may include spirometry sensors, flow sensors, pressure sensors, oxygen and/or carbon dioxide sensors such as, for example, one or more of pulse oximetry sensors, oxygenation sensors (e.g., muscle oxygenation/pH), 02 gas sensors and capnography sensors, impedance sensors, and combinations thereof. The temperature sensors 2242 may include an infrared thermometer, a contact thermometer, a remote thermometer, a liquid crystal thermometer, a thermocouple, a thermistor, etc. and may measure patient temperature internally and/or externally. The chest compression sensor 2244 may include one or more motion sensors including, for example, one or more accelerometers, one or more force sensors, one or more magnetic sensors, one or more velocity sensors, one or more displacement sensors, etc. The chest compression sensor 2244 may provide one or more signals indicative of the chest motion to the medical device 2202 via a wired and/or wireless connection. The chest compression sensor 2244 may be, for example, but not limited to, a compression puck, a smart phone, a hand-held device, a wearable device, etc. The chest compression sensor 2244 may be configured to detect chest motion imparted by a rescuer and/or an automated chest compression device (e.g., a belt-based system, a piston-based system, etc.). The chest compression sensor 2244 may provide signals indicative of chest compression data including displacement data, velocity data, release velocity data, acceleration data, force data, compression rate data, dwell time data, hold time data, blood flow data, blood pressure data, etc. In an implementation, the defibrillation and/or pacing electrodes may include or be configured to couple to the chest compression sensor 2244.

In various implementations, the sensors 2232b and 2236 may include one or more sensor devices configured to provide sensor data that includes, for example, but not limited to electrocardiogram (ECG), blood pressure, heart rate, respiration rate, heart sounds, lung sounds, respiration sounds, end tidal CO₂, saturation of muscle oxygen (SMO₂), oxygen saturation (e.g., SpO₂ and/or PaO₂), cerebral blood flow, point of care laboratory measurements (e.g., lactate, glucose, etc.), temperature, electroencephalogram (EEG) signals, brain oxygen level, tissue pH, tissue fluid levels, images and/or videos via ultrasound, laryngoscopy, and/or other medical imaging techniques, near-infrared spectroscopy, pneumography, cardiography, and/or patient movement. Images and/or videos may be two-dimensional or three-dimensional, such a various forms of ultrasound imaging.

The one or more therapy delivery components 2232a may include electrotherapy electrodes (e.g., the electrotherapy electrodes 2238a), ventilation device(s) (e.g., the ventilation devices 2238b), intravenous device(s) (e.g., the intravenous devices 2238c), compression device(s) (e.g., the compression devices 2238d), etc. For example, the electrotherapy electrodes 2238a may include defibrillation electrodes, pacing electrodes, and combinations thereof. The ventilation devices 2238b may include a tube, a mask, an abdominal and/or chest compressor (e.g., a belt, a cuirass, etc.), etc. and combinations thereof. The intravenous devices 2238c may include drug delivery devices, fluid delivery devices, and combinations thereof. The compression devices 2238d may include mechanical compression devices such as abdominal compressors, chest compressors, belts, pistons, and combinations thereof. In various implementation, the therapy delivery component(s) 2232a may be configured to provide sensor data and/or be coupled to and/or incorporate sensors. For example, the electrotherapy electrodes 2238a may provide sensor data such as transthoracic impedance, ECG, heart rate, etc. Further the electrotherapy electrodes 2238a may include and or be coupled to a chest compression sensor. As another example, the ventilation devices 2238b may be coupled to and/or incorporate flow sensors, gas species sensors (e.g., oxygen sensor, carbon dioxide sensor, etc.), etc. As a further example, the intravenous devices 2238c may be coupled to and/or incorporate temperature sensors, flow sensors, blood pressure sensors, etc. As yet another example, the compression devices 2238d may be coupled to and/or incorporate chest compression sensors, patient position sensors, etc. The therapy delivery control modules 2218 may be configured to couple to and control the therapy delivery component(s) 2232a, respectively.

The one or more sensor(s) 2232b and 2236 and/or the therapy delivery component(s) 2232a may provide sensor data. The patient data provided at the display screens of the medical device 2202 and mobile device 2204 may display the sensor data. For example, the medical device 2202 may process signals received from the sensor(s) 2232b and/or the therapy delivery component(s) 2232a to determine the sensor data. Similarly, the mobile device 2204 may process signals received from the sensor(s) 2236 and/or sensor data from the sensors 2232b received via the medical device 2202 to determine the sensor data.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions, and changes in the form of the methods, apparatuses and systems described herein can be made without departing from the spirit of the present disclosures. The accompanying claims and their equivalents are intended to cover such forms or modifications as would fall within the scope and spirit of the present disclosures.

1-81. (canceled)

82. A context sensitive guidance (CSG) system for guiding caregivers providing medical care for a trauma victim comprises:
a trauma CSG engine comprising hardware logic and/or software logic;
a plurality of contextual data sources configured to communicatively couple to the trauma CSG engine and comprising:
at least one medical device configured to:
collect physiologic data for the trauma victim during the medical care, and
provide the physiologic data to the trauma CSG engine; and
at least one emergency environment data source comprising a mobile computing device configured to:
capture caregiver observations at a touchscreen disposed at the mobile computing device during the medical care, and
provide the caregiver observations to the trauma CSG engine,
wherein the trauma CSG engine is configured to:
receive the physiologic data and the caregiver observations,
evaluate a plurality of protocols based on the physiologic data and the caregiver observations, wherein the plurality of protocols comprises at least one trauma protocol,
select at least one action item for the medical care based on the evaluation of the at least one trauma protocol,
generate at least one caregiver instruction based on the at least one action item, and
provide the at least one caregiver instruction to the mobile computing device for display at the touchscreen.

83.-86. (canceled)

87. The CSG system of claim 82, wherein the at least one medical device comprises at least one of a trauma kit, an automated compression device, a defibrillator, an ultrasound imaging device, or a patient monitor.

88. The CSG system of claim 87, wherein the defibrillator comprises an advanced life support (ALS) defibrillator coupled wirelessly to the mobile computing device, wherein the mobile computing device comprises a companion tablet device that is pre-configured to communicatively couple with the ALS defibrillator and to provide a view of a user interface of the ALS defibrillator in real-time at a display screen disposed at the tablet computing device.

89.-94. (canceled)

95. The CSG system of claim 82, wherein the CSG engine is configured to request one or more of medical device inventory information for a patient care environment or caregiver skill information from a caregiver at the touchscreen.

96. (canceled)

97. The CSG system of claim 82, wherein the CSG engine is configured to access one or more of a previously stored medical device inventory or a previously stored caregiver skill record for a patient care environment.

98.-100. (canceled)

101. The CSG system of claim 82, wherein the plurality of protocols comprise at least a pneumothorax protocol and a cardiac tamponade protocol.

102. The CSG system of claim 101, wherein the at least one action item comprises one of needle decompression, fluid administration, ventilation, or vasopressor administration.

103. The CSG system of claim 82, wherein the plurality of protocols comprises a bleeding protocol.

104. The CSG system of claim 103, wherein the at least one action item comprises one of a tourniquet application or a packing/spray foam administration.

105. The CSG system of claim 82, wherein the CSG engine is configured to:
provide a trauma CSG user interface (UI) at the touchscreen in response to a user selection of a trauma CSG UI tab, and
provide instructions for the at least one action item at the trauma CSG UI.

106. (canceled)

107. The CSG system of claim 105, wherein the trauma CSG UI is configured to provide a device view window for the at least one medical device communicatively coupled to the CSG system.

108. The CSG system of claim 107, wherein the device view window includes one or more source indicators that show a source of a particular item of information in the device view window.

109. The CSG system of claim 105, wherein the trauma CSG engine is configured to provide guidance selection controls at the trauma CSG UI in conjunction with the instructions for the at least one action item.

110. The CSG system of claim 109, wherein the guidance selection controls enable a caregiver to select a level of detail of the provided instructions.

111. The CSG system of claim 109, wherein the guidance selection controls comprise at least one of a continue instructions control, an exit instructions control, a proceed to a next step control, an increase a detail level for guidance control, and a return to a previous instruction control, and a mute or unmute audible UI output control.

112. The CSG system of claim 109, wherein the guidance selection controls comprise a scrollable notification window.

113. The CSG system of claim 105, wherein the trauma CSG engine is configured to receive a caregiver confirmation at the trauma CSG UI in response to the instructions for the at least one action item.

114. The CSG system of claim 113, wherein the trauma CSG engine is configured to receive an incomplete treatment explanation based on the instructions for the at least one action item.

115. The CSG system of claim 105, wherein the instructions comprise one or more of instructions for at least one of operation or assembly of a medical device or medical device settings.

116. (canceled)

117. The CSG system of claim 105, wherein the trauma CSG engine is configured to provide a medication timer at the CSG UI.

118. The CSG system of claim 117, wherein the CSG engine is configured to provide medication delivery instructions with the medication timer.

119. The CSG system of claim 105, wherein the CSG engine is configured to provide closed loop control of at least one medical device based on the at least one action item.

120. The CSG system of claim 105, wherein the mobile computing device comprises a smartphone.

121.-122. (canceled)

123. The CSG system of claim 105, wherein the mobile computing device provides at least one of a device view window tab, a working view window tab, or a trend view window tab as alternatives to the trauma CSG UI tab.

124. The CSG system of claim 82, wherein the trauma CSG engine is configured to operate in an absence of a network connection between the mobile computing device and a remote computing device.

125. The CSG system of claim 82,
wherein the mobile computing device is configured to communicatively couple to a computing device associated with a telemedicine provider, and
wherein the trauma CSG engine is configured to receive information from the telemedicine provider and evaluate the plurality of protocols based on the information from the telemedicine provider.

126. The CSG system of claim 82,
wherein the mobile computing device comprises a patient charting application, and wherein the trauma CSG engine is configured to receive data from and provide data to the patient charting application,
wherein the trauma CSG engine is configured to provide a connected software window at a CSG UI, and
wherein the connected software window indicates a connection status of the patient charting application.

127. (canceled)

128. The CSG system of claim 82, wherein the trauma CSG engine is configured to provide a code generation control configured to generate one or more of a bar code or QR code comprising one or more of medication information, patient information, emergency event information, software application connectivity information, or device connectivity information.

129. The CSG system of claim 82, wherein the trauma CSG engine is configured to receive emergency event notification information prior to an arrival of the caregivers at an emergency scene.

130. The CSG system of claim 129, wherein the trauma CSG engine is configured to receive the emergency event notification information via caregiver input to the touchscreen.

131. The CSG system of claim 129,
wherein the mobile computing device is communicatively coupled to a computer aided dispatch (CAD) system and is configured to receive, from the CAD system, the emergency event notification information comprising at least one of a mechanism of injury (MOI) or an emergency scene location, and
wherein the trauma CSG engine is configured to generate at least one preliminary caregiver instruction prior to the arrival of the caregivers at the emergency scene based on one or more of the MOI or the emergency scene location.

132.-212. (canceled)
